SEQUENCE LISTING (343 sequences)

SEQ ID NO: 1 - protein, 738 aa, Homo sapiens
  SIGNAL 1..26: Signal peptide
  Human CD31
  DOMAIN 28..601: Extracellular domain
  DOMAIN 35..121: Ig-like 1
  DOMAIN 145..233: Ig-like 2
  DOMAIN 236..315: Ig-like 3
  DOMAIN 328..401: Ig-like 4
  DOMAIN 424..493: Ig-like 5
  DOMAIN 499..591: Ig-like 6
  DOMAIN 602..620: Transmembrane domain
  DOMAIN 621..738: Cytoplasmic domain <s300> NCBI: NP_000433 20200609 1-738
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQVLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 2 - protein, 459 aa, Artificial Sequence
  Human sCD31
NSVDMKSLPDWTVQNGKNLTLQCFADVSTTSHVKPQHQMLFYKDDVLFYNISSMKSTESYFIPEVRIYDSGTYKCTVIVNNKEKTTAEYQVLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLR

SEQ ID NO: 3 - protein, 300 aa, Homo sapiens
  Human CD38 <s300> NCBI: NP_001766 20200524
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 4 - protein, 7 aa, Artificial Sequence
  Human sCD31 peptide
STESYFI

SEQ ID NO: 5 - protein, 9 aa, Artificial Sequence
  Human sCD31 peptide
CSTESYFIC

SEQ ID NO: 6 - protein, 15 aa, Artificial Sequence
  Human sCD31 peptide
NSRDQNFVILEFPVE

SEQ ID NO: 7 - protein, 7 aa, Artificial Sequence
  Human sCD31 peptide
NFVILEF

SEQ ID NO: 8 - protein, 8 aa, Artificial Sequence
  Human sCD31 peptide
NFVILEFP

SEQ ID NO: 9 - protein, 17 aa, Artificial Sequence
  Human sCD31 peptide
CRDQNFVILEFPVEEQC

SEQ ID NO: 10 - protein, 22 aa, Artificial Sequence
  Human sCD31 peptide
CMVKLKREKNPGDQNFVILEFC

SEQ ID NO: 11 - protein, 9 aa, Artificial Sequence
  Human sCD31 peptide
CVEEQDRVC

SEQ ID NO: 12 - protein, 11 aa, Artificial Sequence
  Human sCD31 peptide
CIVVNITELFC

SEQ ID NO: 13 - protein, 22 aa, Artificial Sequence
  Human sCD31 peptide
CVESSRISKVPGIVVNITELFC

SEQ ID NO: 14 - protein, 22 aa, Artificial Sequence
  Human sCD31 peptide
CPPANFTIQKPGTIVSQTQDFC

SEQ ID NO: 15 - protein, 8 aa, Artificial Sequence
  Human sCD31 peptide
CEDVEYQC

SEQ ID NO: 16 - protein, 22 aa, Artificial Sequence
  Human CD31 peptide
CSEVLRVKVIPGVDEVQISILC

SEQ ID NO: 17 - protein, 8 aa, Artificial Sequence
  Human CD31 peptide
RVILAPWK

SEQ ID NO: 18 - protein, 585 aa, Mus musculus
  Murine sCD31
MLLALGLTLVLYASLQAEENSFTINSIHMESLPSWEVMNGQQLTLECLVDISTTSKSRSQHRVLFYKDDAMVYNVTSREHTESYVIPQARVFHSGKYKCTVMLNNKEKTTIEYEVKVHGVSKPKVTLDKKEVTEGGVVTVNCSLQEEKPPIFFKIEKLEVGTKFVKRRIDKTSNENFVLMEFPIEAQDHVLVFRCQAGILSGFKLQESEPIRSEYVTVQESFSTPKFEIKPPGMIIEGDQLHIRCIVQVTHLVQEFTEIIIQKDKAIVATSKQSSEAVYSVMAMVEYSGHYTCKVESNRISKASSIMVNITELFPKPKLEFSSSRLDQGELLDLSCSVSGTPVANFTIQKEETVLSQYQNFSKIAEESDSGEYSCTAGIGKVVKRSGLVPIQVCEMLSKPSIFHDAKSEIIKGHAIGISCQSENGTAPITYHLMKAKSDFQTLEVTSNDPATFTDKPTRDMEYQCRADNCHSHPAVFSEILRVRVIAPVDEVVISILSSNEVQSGSEMVLRCSVKEGTSPITFQFYKEKEDRPFHQAVVNDTQAFWHNKQASKKQEGQYYCTASNRASSMRTSPRSSTLAVRVFL

SEQ ID NO: 19 - protein, 7 aa, Artificial Sequence
  peptide
NLTLQSF

SEQ ID NO: 20 - protein, 7 aa, Artificial Sequence
  peptide
TLQSFAD

SEQ ID NO: 21 - protein, 7 aa, Artificial Sequence
  peptide
LQSFADV

SEQ ID NO: 22 - protein, 7 aa, Artificial Sequence
  peptide
QSFADVS

SEQ ID NO: 23 - protein, 7 aa, Artificial Sequence
  peptide
SFADVST

SEQ ID NO: 24 - protein, 20 aa, Artificial Sequence
  peptide
TTSHVKPQHQMLFYKDDVLF

SEQ ID NO: 25 - protein, 20 aa, Artificial Sequence
  peptide
TSHVKPQHQMLFYKDDVLFY

SEQ ID NO: 26 - protein, 20 aa, Artificial Sequence
  peptide
SHVKPQHQMLFYKDDVLFYN

SEQ ID NO: 27 - protein, 15 aa, Artificial Sequence
  peptide
HVKPQHQMLFYKDDV

SEQ ID NO: 28 - protein, 15 aa, Artificial Sequence
  peptide
VKPQHQMLFYKDDVL

SEQ ID NO: 29 - protein, 20 aa, Artificial Sequence
  peptide
VKPQHQMLFYKDDVLFYNIS

SEQ ID NO: 30 - protein, 15 aa, Artificial Sequence
  peptide
KPQHQMLFYKDDVLF

SEQ ID NO: 31 - protein, 20 aa, Artificial Sequence
  peptide
KPQHQMLFYKDDVLFYNISS

SEQ ID NO: 32 - protein, 15 aa, Artificial Sequence
  peptide
QHQMLFYKDDVLFYN

SEQ ID NO: 33 - protein, 15 aa, Artificial Sequence
  peptide
HQMLFYKDDVLFYNI

SEQ ID NO: 34 - protein, 15 aa, Artificial Sequence
  peptide
QMLFYKDDVLFYNIS

SEQ ID NO: 35 - protein, 7 aa, Artificial Sequence
  peptide
MLFYKDD

SEQ ID NO: 36 - protein, 15 aa, Artificial Sequence
  peptide
MLFYKDDVLFYNISS

SEQ ID NO: 37 - protein, 7 aa, Artificial Sequence
  peptide
LFYKDDV

SEQ ID NO: 38 - protein, 15 aa, Artificial Sequence
  peptide
LFYKDDVLFYNISSM

SEQ ID NO: 39 - protein, 7 aa, Artificial Sequence
  peptide
YKDDVLF

SEQ ID NO: 40 - protein, 20 aa, Artificial Sequence
  peptide
YKDDVLFYNISSMKSTESYF

SEQ ID NO: 41 - protein, 7 aa, Artificial Sequence
  peptide
KDDVLFY

SEQ ID NO: 42 - protein, 20 aa, Artificial Sequence
  peptide
KDDVLFYNISSMKSTESYFI

SEQ ID NO: 43 - protein, 7 aa, Artificial Sequence
  peptide
DDVLFYN

SEQ ID NO: 44 - protein, 20 aa, Artificial Sequence
  peptide
DDVLFYNISSMKSTESYFIP

SEQ ID NO: 45 - protein, 7 aa, Artificial Sequence
  peptide
VLFYNIS

SEQ ID NO: 46 - protein, 15 aa, Artificial Sequence
  peptide
VLFYNISSMKSTESY

SEQ ID NO: 47 - protein, 20 aa, Artificial Sequence
  peptide
VLFYNISSMKSTESYFIPEV

SEQ ID NO: 48 - protein, 15 aa, Artificial Sequence
  peptide
FYNISSMKSTESYFI

SEQ ID NO: 49 - protein, 15 aa, Artificial Sequence
  peptide
YNISSMKSTESYFIP

SEQ ID NO: 50 - protein, 15 aa, Artificial Sequence
  peptide
NISSMKSTESYFIPE

SEQ ID NO: 51 - protein, 15 aa, Artificial Sequence
  peptide
ISSMKSTESYFIPEV

SEQ ID NO: 52 - protein, 15 aa, Artificial Sequence
  peptide
SSMKSTESYFIPEVR

SEQ ID NO: 53 - protein, 15 aa, Artificial Sequence
  peptide
MKSTESYFIPEVRIY

SEQ ID NO: 54 - protein, 7 aa, Artificial Sequence
  peptide
KSTESYF

SEQ ID NO: 55 - protein, 15 aa, Artificial Sequence
  peptide
KSTESYFIPEVRIYD

SEQ ID NO: 56 - protein, 15 aa, Artificial Sequence
  peptide
STESYFIPEVRIYDS

SEQ ID NO: 57 - protein, 7 aa, Artificial Sequence
  peptide
TESYFIP

SEQ ID NO: 58 - protein, 15 aa, Artificial Sequence
  peptide
TESYFIPEVRIYDSG

SEQ ID NO: 59 - protein, 15 aa, Artificial Sequence
  peptide
ESYFIPEVRIYDSGT

SEQ ID NO: 60 - protein, 7 aa, Artificial Sequence
  peptide
SYFIPEV

SEQ ID NO: 61 - protein, 7 aa, Artificial Sequence
  peptide
YFIPEVR

SEQ ID NO: 62 - protein, 15 aa, Artificial Sequence
  peptide
YFIPEVRIYDSGTYK

SEQ ID NO: 63 - protein, 7 aa, Artificial Sequence
  peptide
FIPEVRI

SEQ ID NO: 64 - protein, 7 aa, Artificial Sequence
  peptide
EVRIYDS

SEQ ID NO: 65 - protein, 7 aa, Artificial Sequence
  peptide
VRIYDSG

SEQ ID NO: 66 - protein, 7 aa, Artificial Sequence
  peptide
RIYDSGT

SEQ ID NO: 67 - protein, 7 aa, Artificial Sequence
  peptide
TAEYQVL

SEQ ID NO: 68 - protein, 7 aa, Artificial Sequence
  peptide
AEYQVLV

SEQ ID NO: 69 - protein, 7 aa, Artificial Sequence
  peptide
EYQVLVE

SEQ ID NO: 70 - protein, 20 aa, Artificial Sequence
  peptide
KMVKLKREKNSRDQNFVILE

SEQ ID NO: 71 - protein, 20 aa, Artificial Sequence
  peptide
MVKLKREKNSRDQNFVILEF

SEQ ID NO: 72 - protein, 20 aa, Artificial Sequence
  peptide
KLKREKNSRDQNFVILEFPV

SEQ ID NO: 73 - protein, 20 aa, Artificial Sequence
  peptide
LKREKNSRDQNFVILEFPVE

SEQ ID NO: 74 - protein, 15 aa, Artificial Sequence
  peptide
KREKNSRDQNFVILE

SEQ ID NO: 75 - protein, 15 aa, Artificial Sequence
  peptide
REKNSRDQNFVILEF

SEQ ID NO: 76 - protein, 15 aa, Artificial Sequence
  peptide
EKNSRDQNFVILEFP

SEQ ID NO: 77 - protein, 15 aa, Artificial Sequence
  peptide
KNSRDQNFVILEFPV

SEQ ID NO: 78 - protein, 15 aa, Artificial Sequence
  peptide
RDQNFVILEFPVEEQ

SEQ ID NO: 79 - protein, 7 aa, Artificial Sequence
  peptide
DQNFVIL

SEQ ID NO: 80 - protein, 15 aa, Artificial Sequence
  peptide
DQNFVILEFPVEEQD

SEQ ID NO: 81 - protein, 7 aa, Artificial Sequence
  peptide
QNFVILE

SEQ ID NO: 82 - protein, 15 aa, Artificial Sequence
  peptide
QNFVILEFPVEEQDR

SEQ ID NO: 83 - protein, 15 aa, Artificial Sequence
  peptide
NFVILEFPVEEQDRV

SEQ ID NO: 84 - protein, 15 aa, Artificial Sequence
  peptide
FVILEFPVEEQDRVL

SEQ ID NO: 85 - protein, 7 aa, Artificial Sequence
  peptide
VILEFPV

SEQ ID NO: 86 - protein, 7 aa, Artificial Sequence
  peptide
ILEFPVE

SEQ ID NO: 87 - protein, 7 aa, Artificial Sequence
  peptide
VEEQDRV

SEQ ID NO: 88 - protein, 15 aa, Artificial Sequence
  peptide
STIQVTHLAQEFPEI

SEQ ID NO: 89 - protein, 15 aa, Artificial Sequence
  peptide
TIQVTHLAQEFPEII

SEQ ID NO: 90 - protein, 15 aa, Artificial Sequence
  peptide
IQVTHLAQEFPEIII

SEQ ID NO: 91 - protein, 15 aa, Artificial Sequence
  peptide
QVTHLAQEFPEIIIQ

SEQ ID NO: 92 - protein, 15 aa, Artificial Sequence
  peptide
VTHLAQEFPEIIIQK

SEQ ID NO: 93 - protein, 7 aa, Artificial Sequence
  peptide
LAQEFPE

SEQ ID NO: 94 - protein, 7 aa, Artificial Sequence
  peptide
AQEFPEI

SEQ ID NO: 95 - protein, 7 aa, Artificial Sequence
  peptide
QEFPEII

SEQ ID NO: 96 - protein, 7 aa, Artificial Sequence
  peptide
FPEIIIQ

SEQ ID NO: 97 - protein, 20 aa, Artificial Sequence
  peptide
SKVESSRISKVSSIVVNITE

SEQ ID NO: 98 - protein, 20 aa, Artificial Sequence
  peptide
VESSRISKVSSIVVNITELF

SEQ ID NO: 99 - protein, 20 aa, Artificial Sequence
  peptide
ESSRISKVSSIVVNITELFS

SEQ ID NO: 100 - protein, 15 aa, Artificial Sequence
  peptide
SSRISKVSSIVVNIT

SEQ ID NO: 101 - protein, 15 aa, Artificial Sequence
  peptide
RISKVSSIVVNITEL

SEQ ID NO: 102 - protein, 15 aa, Artificial Sequence
  peptide
ISKVSSIVVNITELF

SEQ ID NO: 103 - protein, 15 aa, Artificial Sequence
  peptide
SKVSSIVVNITELFS

SEQ ID NO: 104 - protein, 15 aa, Artificial Sequence
  peptide
KVSSIVVNITELFSK

SEQ ID NO: 105 - protein, 7 aa, Artificial Sequence
  peptide
SSIVVNI

SEQ ID NO: 106 - protein, 7 aa, Artificial Sequence
  peptide
SIVVNIT

SEQ ID NO: 107 - protein, 7 aa, Artificial Sequence
  peptide
IVVNITE

SEQ ID NO: 108 - protein, 9 aa, Artificial Sequence
  peptide
IVVNITELF

SEQ ID NO: 109 - protein, 7 aa, Artificial Sequence
  peptide
VNITELF

SEQ ID NO: 110 - protein, 7 aa, Artificial Sequence
  peptide
NITELFS

SEQ ID NO: 111 - protein, 20 aa, Artificial Sequence
  peptide
PPANFTIQKEDTIVSQTQDF

SEQ ID NO: 112 - protein, 20 aa, Artificial Sequence
  peptide
PANFTIQKEDTIVSQTQDFT

SEQ ID NO: 113 - protein, 20 aa, Artificial Sequence
  peptide
QIVVCEMLSQPRISYDAQFE

SEQ ID NO: 114 - protein, 20 aa, Artificial Sequence
  peptide
IVVCEMLSQPRISYDAQFEV

SEQ ID NO: 115 - protein, 20 aa, Artificial Sequence
  peptide
VVCEMLSQPRISYDAQFEVI

SEQ ID NO: 116 - protein, 7 aa, Artificial Sequence
  peptide
RISYDAQ

SEQ ID NO: 117 - protein, 7 aa, Artificial Sequence
  peptide
SYDAQFE

SEQ ID NO: 118 - protein, 7 aa, Artificial Sequence
  peptide
YDAQFEV

SEQ ID NO: 119 - protein, 7 aa, Artificial Sequence
  peptide
DAQFEVI

SEQ ID NO: 120 - protein, 15 aa, Artificial Sequence
  peptide
NSNDPAVFKDNPTED

SEQ ID NO: 121 - protein, 15 aa, Artificial Sequence
  peptide
SNDPAVFKDNPTEDV

SEQ ID NO: 122 - protein, 15 aa, Artificial Sequence
  peptide
DPAVFKDNPTEDVEY

SEQ ID NO: 123 - protein, 15 aa, Artificial Sequence
  peptide
PAVFKDNPTEDVEYQ

SEQ ID NO: 124 - protein, 15 aa, Artificial Sequence
  peptide
VFKDNPTEDVEYQCV

SEQ ID NO: 125 - protein, 7 aa, Artificial Sequence
  peptide
NPTEDVE

SEQ ID NO: 126 - protein, 7 aa, Artificial Sequence
  peptide
PTEDVEY

SEQ ID NO: 127 - protein, 6 aa, Artificial Sequence
  peptide
EDVEYQ

SEQ ID NO: 128 - protein, 7 aa, Artificial Sequence
  peptide
EDVEYQC

SEQ ID NO: 129 - protein, 7 aa, Artificial Sequence
  peptide
DVEYQCV

SEQ ID NO: 130 - protein, 9 aa, Artificial Sequence
  peptide
LFYKDDVLF

SEQ ID NO: 131 - protein, 11 aa, Artificial Sequence
  peptide
LFYKDDVLFYN

SEQ ID NO: 132 - protein, 17 aa, Artificial Sequence
  peptide
VLFYNISSMKSTESYFI

SEQ ID NO: 133 - protein, 6 aa, Artificial Sequence
  peptide
TESYFI

SEQ ID NO: 134 - protein, 13 aa, Artificial Sequence
  peptide
TESYFIPEVRIYD

SEQ ID NO: 135 - protein, 5 aa, Artificial Sequence
  peptide
FIPEV

SEQ ID NO: 136 - protein, 6 aa, Artificial Sequence
  peptide
VRIYDS

SEQ ID NO: 137 - protein, 5 aa, Artificial Sequence
  peptide
EYQVL

SEQ ID NO: 138 - protein, 13 aa, Artificial Sequence
  peptide
RDQNFVILEFPVE

SEQ ID NO: 139 - protein, 12 aa, Artificial Sequence
  peptide
DQNFVILEFPVE

SEQ ID NO: 140 - protein, 6 aa, Artificial Sequence
  peptide
NFVILE

SEQ ID NO: 141 - protein, 14 aa, Artificial Sequence
  peptide
QVTHLAQEFPEIII

SEQ ID NO: 142 - protein, 4 aa, Artificial Sequence
  peptide
FPEI

SEQ ID NO: 143 - protein, 17 aa, Artificial Sequence
  peptide
ESSRISKVSSIVVNITE

SEQ ID NO: 144 - protein, 14 aa, Artificial Sequence
  peptide
ISKVSSIVVNITEL

SEQ ID NO: 145 - protein, 13 aa, Artificial Sequence
  peptide
SKVSSIVVNITEL

SEQ ID NO: 146 - protein, 6 aa, Artificial Sequence
  peptide
NITELF

SEQ ID NO: 147 - protein, 5 aa, Artificial Sequence
  peptide
DAQFE

SEQ ID NO: 148 - protein, 9 aa, Artificial Sequence
  peptide
VFKDNPTED

SEQ ID NO: 149 - protein, 6 aa, Artificial Sequence
  peptide
DVEYQC

SEQ ID NO: 150 - protein, 14 aa, Artificial Sequence
  peptide
TESYFIPEVRIYDS

SEQ ID NO: 151 - protein, 23 aa, Artificial Sequence
  peptide
MVKLKREKNSRDQNFVILEFPVE

SEQ ID NO: 152 - protein, 18 aa, Artificial Sequence
  peptide
ESSRISKVSSIVVNITEL

SEQ ID NO: 153 - protein, 9 aa, Artificial Sequence
  peptide
VVSEMLSQP

SEQ ID NO: 154 - protein, 14 aa, Artificial Sequence
  peptide
VFKDNPTEDVEYQS

SEQ ID NO: 155 - protein, 11 aa, Artificial Sequence
  peptide
CLFYKDDVLFC

SEQ ID NO: 156 - protein, 16 aa, Artificial Sequence
  peptide
CTESYFIPEVRIYDSC

SEQ ID NO: 157 - protein, 25 aa, Artificial Sequence
  peptide
CMVKLKREKNSRDQNFVILEFPVEC

SEQ ID NO: 158 - protein, 16 aa, Artificial Sequence
  peptide
CQVTHLAQEFPEIIIC

SEQ ID NO: 159 - protein, 20 aa, Artificial Sequence
  peptide
CESSRISKVSSIVVNITELC

SEQ ID NO: 160 - protein, 11 aa, Artificial Sequence
  peptide
CVVSEMLSQPC

SEQ ID NO: 161 - protein, 7 aa, Artificial Sequence
  peptide
CDAQFEC

SEQ ID NO: 162 - protein, 16 aa, Artificial Sequence
  peptide
CVFKDNPTEDVEYQSC

SEQ ID NO: 163 - protein, 22 aa, Artificial Sequence
  peptide
CMVKLKREKNSRDQNFVILEFC

SEQ ID NO: 164 - protein, 17 aa, Artificial Sequence
  peptide
CNSRDQNFVILEFPVEC

SEQ ID NO: 165 - protein, 22 aa, Artificial Sequence
  peptide
CVESSRISKVSSIVVNITELFC

SEQ ID NO: 166 - protein, 22 aa, Artificial Sequence
  peptide
CPPANFTIQKEDTIVSQTQDFC

SEQ ID NO: 167 - protein, 9 aa, Artificial Sequence
  peptide
CEDVEYQSC

SEQ ID NO: 168 - protein, 15 aa, Artificial Sequence
  peptide
KQKASKEQEGEYYCT

SEQ ID NO: 169 - protein, 15 aa, Artificial Sequence
  peptide
QKASKEQEGEYYCTA

SEQ ID NO: 170 - protein, 15 aa, Artificial Sequence
  peptide
ASKEQEGEYYCTAFN

SEQ ID NO: 171 - protein, 15 aa, Artificial Sequence
  peptide
SKEQEGEYYCTAFNR

SEQ ID NO: 172 - protein, 15 aa, Artificial Sequence
  peptide
EQEGEYYCTAFNRAN

SEQ ID NO: 173 - protein, 9 aa, Artificial Sequence
  peptide
CNLTLQSFC

SEQ ID NO: 174 - protein, 9 aa, Artificial Sequence
  peptide
CTLQSFADC

SEQ ID NO: 175 - protein, 9 aa, Artificial Sequence
  peptide
CLQSFADVC

SEQ ID NO: 176 - protein, 9 aa, Artificial Sequence
  peptide
CQSFADVSC

SEQ ID NO: 177 - protein, 9 aa, Artificial Sequence
  peptide
CSFADVSTC

SEQ ID NO: 178 - protein, 9 aa, Artificial Sequence
  peptide
CMLFYKDDC

SEQ ID NO: 179 - protein, 9 aa, Artificial Sequence
  peptide
CLFYKDDVC

SEQ ID NO: 180 - protein, 9 aa, Artificial Sequence
  peptide
CYKDDVLFC

SEQ ID NO: 181 - protein, 9 aa, Artificial Sequence
  peptide
CKDDVLFYC

SEQ ID NO: 182 - protein, 9 aa, Artificial Sequence
  peptide
CDDVLFYNC

SEQ ID NO: 183 - protein, 9 aa, Artificial Sequence
  peptide
CVLFYNISC

SEQ ID NO: 184 - protein, 9 aa, Artificial Sequence
  peptide
CKSTESYFC

SEQ ID NO: 185 - protein, 9 aa, Artificial Sequence
  peptide
CTESYFIPC

SEQ ID NO: 186 - protein, 9 aa, Artificial Sequence
  peptide
CSYFIPEVC

SEQ ID NO: 187 - protein, 9 aa, Artificial Sequence
  peptide
CYFIPEVRC

SEQ ID NO: 188 - protein, 9 aa, Artificial Sequence
  peptide
CFIPEVRIC

SEQ ID NO: 189 - protein, 9 aa, Artificial Sequence
  peptide
CEVRIYDSC

SEQ ID NO: 190 - protein, 9 aa, Artificial Sequence
  peptide
CVRIYDSGC

SEQ ID NO: 191 - protein, 9 aa, Artificial Sequence
  peptide
CRIYDSGTC

SEQ ID NO: 192 - protein, 9 aa, Artificial Sequence
  peptide
CTAEYQVLC

SEQ ID NO: 193 - protein, 9 aa, Artificial Sequence
  peptide
CAEYQVLVC

SEQ ID NO: 194 - protein, 9 aa, Artificial Sequence
  peptide
CEYQVLVEC

SEQ ID NO: 195 - protein, 9 aa, Artificial Sequence
  peptide
CDQNFVILC

SEQ ID NO: 196 - protein, 9 aa, Artificial Sequence
  peptide
CQNFVILEC

SEQ ID NO: 197 - protein, 9 aa, Artificial Sequence
  peptide
CVILEFPVC

SEQ ID NO: 198 - protein, 9 aa, Artificial Sequence
  peptide
CILEFPVEC

SEQ ID NO: 199 - protein, 9 aa, Artificial Sequence
  peptide
CLAQEFPEC

SEQ ID NO: 200 - protein, 9 aa, Artificial Sequence
  peptide
CAQEFPEIC

SEQ ID NO: 201 - protein, 9 aa, Artificial Sequence
  peptide
CQEFPEIIC

SEQ ID NO: 202 - protein, 9 aa, Artificial Sequence
  peptide
CFPEIIIQC

SEQ ID NO: 203 - protein, 9 aa, Artificial Sequence
  peptide
CSSIVVNIC

SEQ ID NO: 204 - protein, 9 aa, Artificial Sequence
  peptide
CSIVVNITC

SEQ ID NO: 205 - protein, 9 aa, Artificial Sequence
  peptide
CIVVNITEC

SEQ ID NO: 206 - protein, 9 aa, Artificial Sequence
  peptide
CVNITELFC

SEQ ID NO: 207 - protein, 9 aa, Artificial Sequence
  peptide
CNITELFSC

SEQ ID NO: 208 - protein, 9 aa, Artificial Sequence
  peptide
CRISYDAQC

SEQ ID NO: 209 - protein, 9 aa, Artificial Sequence
  peptide
CSYDAQFEC

SEQ ID NO: 210 - protein, 9 aa, Artificial Sequence
  peptide
CYDAQFEVC

SEQ ID NO: 211 - protein, 9 aa, Artificial Sequence
  peptide
CDAQFEVIC

SEQ ID NO: 212 - protein, 9 aa, Artificial Sequence
  peptide
CNPTEDVEC

SEQ ID NO: 213 - protein, 9 aa, Artificial Sequence
  peptide
CPTEDVEYC

SEQ ID NO: 214 - protein, 9 aa, Artificial Sequence
  peptide
CDVEYQSVC

SEQ ID NO: 215 - protein, 9 aa, Artificial Sequence
  peptide
CDEVQISIC

SEQ ID NO: 216 - protein, 9 aa, Artificial Sequence
  peptide
CEVQISILC

SEQ ID NO: 217 - protein, 9 aa, Artificial Sequence
  peptide
CVQISILSC

SEQ ID NO: 218 - protein, 9 aa, Artificial Sequence
  peptide
CESGEDIVC

SEQ ID NO: 219 - protein, 9 aa, Artificial Sequence
  peptide
CSGEDIVLC

SEQ ID NO: 220 - protein, 9 aa, Artificial Sequence
  peptide
CGEDIVLQC

SEQ ID NO: 221 - protein, 9 aa, Artificial Sequence
  peptide
CDIVLQSAC

SEQ ID NO: 222 - protein, 9 aa, Artificial Sequence
  peptide
CKEQEGEYC

SEQ ID NO: 223 - protein, 9 aa, Artificial Sequence
  peptide
CQEGEYYSC

SEQ ID NO: 224 - protein, 9 aa, Artificial Sequence
  peptide
CEGEYYSTC

SEQ ID NO: 225 - protein, 9 aa, Artificial Sequence
  peptide
CGEYYSTAC

SEQ ID NO: 226 - protein, 9 aa, Artificial Sequence
  peptide
CYYSTAFNC

SEQ ID NO: 227 - protein, 17 aa, Artificial Sequence
  peptide
CHVKPQHQMLFYKDDVC

SEQ ID NO: 228 - protein, 17 aa, Artificial Sequence
  peptide
CVKPQHQMLFYKDDVLC

SEQ ID NO: 229 - protein, 17 aa, Artificial Sequence
  peptide
CKPQHQMLFYKDDVLFC

SEQ ID NO: 230 - protein, 17 aa, Artificial Sequence
  peptide
CQHQMLFYKDDVLFYNC

SEQ ID NO: 231 - protein, 17 aa, Artificial Sequence
  peptide
CHQMLFYKDDVLFYNIC

SEQ ID NO: 232 - protein, 17 aa, Artificial Sequence
  peptide
CQMLFYKDDVLFYNISC

SEQ ID NO: 233 - protein, 17 aa, Artificial Sequence
  peptide
CMLFYKDDVLFYNISSC

SEQ ID NO: 234 - protein, 17 aa, Artificial Sequence
  peptide
CLFYKDDVLFYNISSMC

SEQ ID NO: 235 - protein, 17 aa, Artificial Sequence
  peptide
CVLFYNISSMKSTESYC

SEQ ID NO: 236 - protein, 17 aa, Artificial Sequence
  peptide
CFYNISSMKSTESYFIC

SEQ ID NO: 237 - protein, 17 aa, Artificial Sequence
  peptide
CYNISSMKSTESYFIPC

SEQ ID NO: 238 - protein, 17 aa, Artificial Sequence
  peptide
CNISSMKSTESYFIPEC

SEQ ID NO: 239 - protein, 17 aa, Artificial Sequence
  peptide
CISSMKSTESYFIPEVC

SEQ ID NO: 240 - protein, 17 aa, Artificial Sequence
  peptide
CSSMKSTESYFIPEVRC

SEQ ID NO: 241 - protein, 17 aa, Artificial Sequence
  peptide
CMKSTESYFIPEVRIYC

SEQ ID NO: 242 - protein, 17 aa, Artificial Sequence
  peptide
CKSTESYFIPEVRIYDC

SEQ ID NO: 243 - protein, 17 aa, Artificial Sequence
  peptide
CSTESYFIPEVRIYDSC

SEQ ID NO: 244 - protein, 17 aa, Artificial Sequence
  peptide
CTESYFIPEVRIYDSGC

SEQ ID NO: 245 - protein, 17 aa, Artificial Sequence
  peptide
CESYFIPEVRIYDSGTC

SEQ ID NO: 246 - protein, 17 aa, Artificial Sequence
  peptide
CYFIPEVRIYDSGTYKC

SEQ ID NO: 247 - protein, 17 aa, Artificial Sequence
  peptide
CKREKNSRDQNFVILEC

SEQ ID NO: 248 - protein, 17 aa, Artificial Sequence
  peptide
CREKNSRDQNFVILEFC

SEQ ID NO: 249 - protein, 17 aa, Artificial Sequence
  peptide
CEKNSRDQNFVILEFPC

SEQ ID NO: 250 - protein, 17 aa, Artificial Sequence
  peptide
CKNSRDQNFVILEFPVC

SEQ ID NO: 251 - protein, 17 aa, Artificial Sequence
  peptide
CDQNFVILEFPVEEQDC

SEQ ID NO: 252 - protein, 17 aa, Artificial Sequence
  peptide
CQNFVILEFPVEEQDRC

SEQ ID NO: 253 - protein, 17 aa, Artificial Sequence
  peptide
CNFVILEFPVEEQDRVC

SEQ ID NO: 254 - protein, 17 aa, Artificial Sequence
  peptide
CFVILEFPVEEQDRVLC

SEQ ID NO: 255 - protein, 17 aa, Artificial Sequence
  peptide
CSTIQVTHLAQEFPEIC

SEQ ID NO: 256 - protein, 17 aa, Artificial Sequence
  peptide
CTIQVTHLAQEFPEIIC

SEQ ID NO: 257 - protein, 17 aa, Artificial Sequence
  peptide
CIQVTHLAQEFPEIIIC

SEQ ID NO: 258 - protein, 17 aa, Artificial Sequence
  peptide
CQVTHLAQEFPEIIIQC

SEQ ID NO: 259 - protein, 17 aa, Artificial Sequence
  peptide
CVTHLAQEFPEIIIQKC

SEQ ID NO: 260 - protein, 17 aa, Artificial Sequence
  peptide
CSSRISKVSSIVVNITC

SEQ ID NO: 261 - protein, 17 aa, Artificial Sequence
  peptide
CRISKVSSIVVNITELC

SEQ ID NO: 262 - protein, 17 aa, Artificial Sequence
  peptide
CISKVSSIVVNITELFC

SEQ ID NO: 263 - protein, 17 aa, Artificial Sequence
  peptide
CSKVSSIVVNITELFSC

SEQ ID NO: 264 - protein, 17 aa, Artificial Sequence
  peptide
CKVSSIVVNITELFSKC

SEQ ID NO: 265 - protein, 22 aa, Artificial Sequence
  peptide
CTTSHVKPQHPGLFYKDDVLFC

SEQ ID NO: 266 - protein, 22 aa, Artificial Sequence
  peptide
CTSHVKPQHQPGFYKDDVLFYC

SEQ ID NO: 267 - protein, 22 aa, Artificial Sequence
  peptide
CSHVKPQHQMPGYKDDVLFYNC

SEQ ID NO: 268 - protein, 22 aa, Artificial Sequence
  peptide
CVKPQHQMLFPGDDVLFYNISC

SEQ ID NO: 269 - protein, 22 aa, Artificial Sequence
  peptide
CKPQHQMLFYPGDVLFYNISSC

SEQ ID NO: 270 - protein, 22 aa, Artificial Sequence
  peptide
CYKDDVLFYNPGSMKSTESYFC

SEQ ID NO: 271 - protein, 22 aa, Artificial Sequence
  peptide
CKDDVLFYNIPGMKSTESYFIC

SEQ ID NO: 272 - protein, 22 aa, Artificial Sequence
  peptide
CDDVLFYNISPGKSTESYFIPC

SEQ ID NO: 273 - protein, 22 aa, Artificial Sequence
  peptide
CVLFYNISSMPGTESYFIPEVC

SEQ ID NO: 274 - protein, 22 aa, Artificial Sequence
  peptide
CKMVKLKREKPGRDQNFVILEC

SEQ ID NO: 275 - protein, 22 aa, Artificial Sequence
  peptide
CKLKREKNSRPGNFVILEFPVC

SEQ ID NO: 276 - protein, 22 aa, Artificial Sequence
  peptide
CLKREKNSRDPGFVILEFPVEC

SEQ ID NO: 277 - protein, 22 aa, Artificial Sequence
  peptide
CSKVESSRISPGSSIVVNITEC

SEQ ID NO: 278 - protein, 22 aa, Artificial Sequence
  peptide
CESSRISKVSPGVVNITELFSC

SEQ ID NO: 279 - protein, 22 aa, Artificial Sequence
  peptide
CPANFTIQKEPGIVSQTQDFTC

SEQ ID NO: 280 - protein, 22 aa, Artificial Sequence
  peptide
CQIVVSEMLSPGRISYDAQFEC

SEQ ID NO: 281 - protein, 22 aa, Artificial Sequence
  peptide
CIVVSEMLSQPGISYDAQFEVC

SEQ ID NO: 282 - protein, 22 aa, Artificial Sequence
  peptide
CVVSEMLSQPPGSYDAQFEVIC

SEQ ID NO: 283 - protein, 22 aa, Artificial Sequence
  peptide
CLSEVLRVKVPGPVDEVQISIC

SEQ ID NO: 284 - protein, 22 aa, Artificial Sequence
  peptide
CEVLRVKVIAPGDEVQISILSC

SEQ ID NO: 285 - protein, 7 aa, Artificial Sequence
  Scramble peptide
FELIVFN

SEQ ID NO: 286 - protein, 14 aa, Artificial Sequence
  peptide
NSRDQNFVILEFPV

SEQ ID NO: 287 - protein, 13 aa, Artificial Sequence
  peptide
NSRDQNFVILEFP

SEQ ID NO: 288 - protein, 12 aa, Artificial Sequence
  peptide
NSRDQNFVILEF

SEQ ID NO: 289 - protein, 11 aa, Artificial Sequence
  peptide
NSRDQNFVILE

SEQ ID NO: 290 - protein, 10 aa, Artificial Sequence
  peptide
NSRDQNFVIL

SEQ ID NO: 291 - protein, 9 aa, Artificial Sequence
  peptide
CNFVILEFC

SEQ ID NO: 292 - protein, 7 aa, Artificial Sequence
  peptide
FVILEFP

SEQ ID NO: 293 - protein, 8 aa, Artificial Sequence
  peptide
AFVILEFP

SEQ ID NO: 294 - protein, 8 aa, Artificial Sequence
  peptide
NAVILEFP

SEQ ID NO: 295 - protein, 8 aa, Artificial Sequence
  peptide
NFAILEFP

SEQ ID NO: 296 - protein, 8 aa, Artificial Sequence
  peptide
NFVALEFP

SEQ ID NO: 297 - protein, 8 aa, Artificial Sequence
  peptide
NFVIAEFP

SEQ ID NO: 298 - protein, 8 aa, Artificial Sequence
  peptide
NFVILAFP

SEQ ID NO: 299 - protein, 8 aa, Artificial Sequence
  peptide
NFVILEAP

SEQ ID NO: 300 - protein, 8 aa, Artificial Sequence
  peptide
NFVILEFA

SEQ ID NO: 301 - protein, 8 aa, Artificial Sequence
  peptide
QFVILEFP

SEQ ID NO: 302 - protein, 8 aa, Artificial Sequence
  peptide
SFVILEFP

SEQ ID NO: 303 - protein, 8 aa, Artificial Sequence
  peptide
TFVILEFP

SEQ ID NO: 304 - protein, 8 aa, Artificial Sequence
  peptide
HFVILEFP

SEQ ID NO: 305 - protein, 8 aa, Artificial Sequence
  peptide
NYVILEFP

SEQ ID NO: 306 - protein, 8 aa, Artificial Sequence
  peptide
NWVILEFP

SEQ ID NO: 307 - protein, 8 aa, Artificial Sequence
  peptride
NIVILEFP

SEQ ID NO: 308 - protein, 8 aa, Artificial Sequence
  peptide
NSVILEFP

SEQ ID NO: 309 - protein, 8 aa, Artificial Sequence
  peptide
NHVILEFP

SEQ ID NO: 310 - protein, 8 aa, Artificial Sequence
  peptide
NDVILEFP

SEQ ID NO: 311 - protein, 8 aa, Artificial Sequence
  peptide
NFVFLEFP

SEQ ID NO: 312 - protein, 8 aa, Artificial Sequence
  peptide
NFVTLEFP

SEQ ID NO: 313 - protein, 8 aa, Artificial Sequence
  peptide
NFVQLEFP

SEQ ID NO: 314 - protein, 8 aa, Artificial Sequence
  peptide
NFVHLEFP

SEQ ID NO: 315 - protein, 8 aa, Artificial Sequence
  peptide
NFVDLEFP

SEQ ID NO: 316 - protein, 8 aa, Artificial Sequence
  peptide
NFVITEFP

SEQ ID NO: 317 - protein, 8 aa, Artificial Sequence
  peptide
NFVIFEFP

SEQ ID NO: 318 - protein, 8 aa, Artificial Sequence
  peptide
NFVIHEFP

SEQ ID NO: 319 - protein, 8 aa, Artificial Sequence
  peptide
NFVIDEFP

SEQ ID NO: 320 - protein, 8 aa, Artificial Sequence
  peptide
NSMLIEFT

SEQ ID NO: 321 - protein, 5 aa, Artificial Sequence
  peptide
NFVIL

SEQ ID NO: 322 - protein, 5 aa, Artificial Sequence
  SITE 1: Xaa is an amino acid residue with a polar uncharged side chain, preferably Xaa is selected from the group consisting of asparagine (Asn, N), glutamine (Gln, Q), serine (Ser, S), and threonine (Thr, T)
  peptide
  SITE 2: Xaa is an aromatic amino acid residue or an amino acid residue wi th a polar uncharged side chain, preferably Xaa is selected from the group consisting of phenylalanine (Phe, F), tyrosine (Tyr, Y) , tryptophan (Trp, W), serine (Ser, S), threonine (Thr, T), aspar
  SITE 3: Xaa is any amino acid residue
  SITE 4: Xaa is an amino acid residue with a hydrophobic side chain or wit h a polar uncharged side chain, preferably Xaa is selected from t he group consisting of isoleucine (Ile, I), alanine (Ala, A), val ine (Val, V), leucine (Leu, L), methionine (Met, M), cysteine (Cy
  SITE 5: Xaa is an amino acid residue with a hydrophobic side chain, prefe rably Xaa is selected from the group consisting of leucine (Leu, L), alanine (Ala, A), valine (Val, V), isoleucine (Ile, I), methi onine (Met, M), cysteine (Cys, C), phenylalanine (Phe, F), tyrosi
XXXXX

SEQ ID NO: 323 - protein, 727 aa, Mus musculus
  CD31
MLLALGLTLVLYASLQAEENSFTINSIHMESLPSWEVMNGQQLTLECLVDISTTSKSRSQHRVLFYKDDAMVYNVTSREHTESYVIPQARVFHSGKYKCTVMLNNKEKTTIEYEVKVHGVSKPKVTLDKKEVTEGGVVTVNCSLQEEKPPIFFKIEKLEVGTKFVKRRIDKTSNENFVLMEFPIEAQDHVLVFRCQAGILSGFKLQESEPIRSEYVTVQESFSTPKFEIKPPGMIIEGDQLHIRCIVQVTHLVQEFTEIIIQKDKAIVATSKQSSEAVYSVMAMVEYSGHYTCKVESNRISKASSIMVNITELFPKPKLEFSSSRLDQGELLDLSCSVSGTPVANFTIQKEETVLSQYQNFSKIAEESDSGEYSCTAGIGKVVKRSGLVPIQVCEMLSKPSIFHDAKSEIIKGHAIGISCQSENGTAPITYHLMKAKSDFQTLEVTSNDPATFTDKPTRDMEYQCRADNCHSHPAVFSEILRVRVIAPVDEVVISILSSNEVQSGSEMVLRCSVKEGTSPITFQFYKEKEDRPFHQAVVNDTQAFWHNKQASKKQEGQYYCTASNRASSMRTSPRSSTLAVRVFLAPWKKGLIAVVVIGVVIATLIVAAKCYFLRKAKAKQKPVEMSRPAAPLLNSNSEKISEPSVEANSHYGYDDVSGNDAVKPINQNKDPQNMDVEYTEVEVSSLEPHQALGTRATETVYSEIRKVDPNLMENRYSRTEGSLNGT

SEQ ID NO: 324 - protein, 678 aa, Rattus norvegicus
  CD31
MLLALLLTMLLYASLQAQENSFTINSIHMESRPSWEVSNGQKLTLQCLVDISTTSKSRPQHQVLFYKDDALVYNVSSSEHTESFVIPQSRVFHAGKYKCTVILNSKEKTTIEYQLTVNGVPMPEVTVDKKEVTEGGIVTVNCSMQEEKPPIYFKIEKVELGTKNVKLSREKTSNMNFVLIEFPIEEQDHLLVFRCQAGVLSGIKMQTSEFIRSEYVTVQEFFSTPKFQIQPPEMIIEGNQLHIKCSVQVAHLAQEFPEIIIQKDKAIVATSKQSKEAVYSVMALVEHSGHYTCKVESNRISKASSILVNITELFPRPKLELSSSRLDQGEMLDLSCSVSGAPVANFTIQKEETVLSQYQNFSKIAEERDSGLYSCTAGIGKVVKRSNLVPVQVCEMLSKPRIFHDAKFEIIKGQIIGISCQSVNGTAPITYRLLRAKSNFQTVQKNSNDPVTFTDKPTRDMEYQCIVDNCHSHPEVRSEILRVKVIAPVDEVTISILSGNDVQSGDEMVLRCSVKEGTGPVTFQFYKEKEGRPFHEETVNDTQVFWHHEQTSKEQEGQYYCTAFNRASIVTSLRSGPLTVRVFLAPWKKGLIAVVVIGVVIAALIVAAKYYFLRKAKAKQKPVEMSRPAVPLLNSNSEKVSEPSVETNSHYDSQNMDVEYTEVEVSSLEPHQENGRLP

SEQ ID NO: 325 - protein, 764 aa, Felis catus
  CD31
MQPETAQDGKMWLGVLLALMLCPGLEGQENPFTINYIHMNILPGLEVQNGENLTLQCVVDISTTSHVKPQHWVLFYKDDVLFHNVSSMKNTESYFIPQARVCDAGTYKCTVILNNKEKTTLEYQVWVKGVSDPRVTLDKKEAIEGGVVTVNCSVPEENPPIYFTIEKLKLDAKGFKQKREKVSQNQNFMVMEFTVEEQDHVILFQCQARIFSGTNVEISKAIRSELVTVTESFSNPRFHISPKGVITEGDQLNIKCTIQVTHLVQSFPEIIIQKDKAIVAHKTHGNEATYSVMAMVEHSGNYTCKVEASRISKVSSIVVNITELFSKPKLESSATRLDHGESLNLWCSIPGAPPANFTIQKENTIVSLSQNFTKIASERDRGTYTCNAGIGKVVKKSNAVQITVCEMLSKPRIFYDSGPEVIKGQTIAVSCQSVNGTAPISYHLVKANSILESCNMDSNEPAVFKDKPTKDVEYQCIADNCHSHTEMISEVLRVKVIAPVDQVKLTILLNEEVESGKEVVLRCSVNDASGPITYRFYREKEGSPFYQIISNDTYAIWHKDKASKEHEGQYYCTAFNRANLVKSNPQSNILTVRVFLAPWKKGLIAVVVIGVIIATLILGARCCFLKKAKAKQTPVEMARPAAPLLNSSNEKMLSDPDTEANRHYGYSEDVGNHAMKPANENKEPLTMDVEYTEVQVTPSEPYQEKNRSQNKAPSGNIPDSRDKQPAGLGLETKGTETVYSEIRKTNPDFVENRYSRTEGSLDGT

SEQ ID NO: 326 - protein, 803 aa, Canis lupus familiaris
  CD31
MHPGKNQEISLGVLWVEDGKGKELSPCHERGLQIQGGFIWATWEVPSAPGAWPKGRTNCPKFTLKMQLEWTQEGKMWLGALLILLLCPSLEGQENSFTINDIHMEILPGEEVQNGENMTLQCIVDISTTSHIKPQHWVLFYKDDVLFHNVSSVENTESYFIPRARVYDAGTYKCTVILNNKEKTSLEYQVWVKGVSDPIVTLDKKEAIEGGVVKVNCSVPEEKPPIHFTIEKLKLDTKGFKQKREKTSPNRNFMMLEFTVEEQDHVIFFQCQARIISGTHMETSRAIKSELVTVTESFSNPKFHVSPEGVITEGDQLYIRCTIQVTHLVQAFPEIIIQKDKAIVAHKRHGNEATYSVMAMAEHNGNYTCKVEASRISKVSSIVVNITELFSKPKLESSITRLDQGESLNLWCSIPEAPPANFTIQKENTIVSQSQNFTKIASATDSGTYTCNASMGKVVKRSSAVQITVCEMLSKPRIFYDSSSEVIKGQTIAVSCQSINGTTPISYHLLKTSNILESRDMSSNEPAVFKDNPTKDTEYQCIVDNCHSHSEMVSEVLRVKVIAPVDEVKLSILMNAEVEFGKDIELLCSVNEATGPITYRFYKEAGSLLYQITSNETHAVWYKSKASKEDEGQYYCTASNRANRLKSSPQSNVLTVRVFLAAWIKGLIAVVVIGVIIGVLILGARCYILKKAKAKQTPVEMSRPAVPLLNSTNEKMLSDPNTEVNRHYGYNEDVGNHAMKPINENKEPLTLDVEYTEVEVTSPEPYQGLETKGTETVYSEIRKANPDFMENRYSRTEGSLDGT

SEQ ID NO: 327 - protein, 812 aa, Ovis aries
  CD31
MRPPRHCSWFPAWPPLPDQRLGQCVRSAWRGSQSWSLLFTGRVSQLRLQRRVFCPPGHGPNAELIAKVSAPLRMQLRWTQRGMMWLGALLTLLLCSSLKGQENSFTINSIHMQILPQNTVQNGENLTLQCLVDVSTTSPVQPLHQVLFYKDDVLLRNISSRTNRESYLIPHVRVCDSGRYKCTVKLNNKEKATPEYEVWVKGVSDPRVTLDKKEVIEGGVVVVNCSVPEEKAPVHFTIEKFELNIRSAKKKREKTSQNQNFVTLEFTVEEQDRAIFFQCQAKIFSGSNVESSRPMRSELVTVRESFSNPKFHIIPEGKITEGDDLQVKCTVQVTHQAQSFPEIIIQKDREIVAHNSLSSEAVYSVMATTEHNGNYTCKVEASRISKVSSIVVNITELFSKPKLESSATHLDQGENLNLWCSIPGAPPANFTIQKGGMTVSQTQNFSKRVAEWDSGLYTCVAGVGRVVKRSNTVQITVCEMLSKPSIFHDSRSEVIKGQTIEVSCQSINGTAPIFYQLSNTSKPVANQSMGSNEPAIFRDKPTKDVEYWCSADNCHSHSKMFSEVLRVKVIAPVDEAKLSILLKEEVESGMPIVLRCSVSEGSSPITYKFYKGKESKPFHQETLNATLAIWHEDTASKEHEGQYYCTASNRANLSKHVIQSNTLTIRVYLAPWKKGLIAVVVIGVIIATLVFGARCYFLKKAKAKQMPVEMSRPAVPLLNSNNEKTLSDASTEADRHYGFNEDVGNHAMKPLNENKEPLTLDVEYTEVEVTSPEPHRGLGTKGTETVYSEIRKADPDFVENRYSRTEGSLNGS

SEQ ID NO: 328 - protein, 740 aa, Sus scrofa
  CD31
MRLRWTQGGNMWLGVLLTLQLCSSLEGQENSFTINSIHMEMLPGQEVHNGENLTLQCIVDVSTTSSVKPQHQVLFYKDDVLFHNVSSTKNTESYFISEARVYNSGRYKCTVILNNKEKTTAEYKVVVEGVSNPRVTLDKKEVIEGGVVKVTCSVPEEKPPVHFIIEKFELNVRDVKQRREKTANNQNSVTLEFTVEEQDRVILFSCQANVIFGTRVEISDSVRSDLVTVRESFSNPKFHISPKGVIIEGDQLLIKCTIQVTHQAQSFPEIIIQKDKEIVAHSRNGSEAVYSVMATVEHNSNYTCKVEASRISKVSSIMVNITELFSRPKLKSSATRLDQGESLRLWCSIPGAPPEANFTIQKGGMMMLQDQNLTKVASERDSGTYTCVAGIGKVVKRSNEVQIAVCEMLSKPSIFHDSGSEVIKGQTIEVSCQSINGTSPISYQLLKGSDLLASQNVSSNEPAVFKDNPTKDVEYQCIADNCHSHAGMPSKVLRVKVIAPVEEVKLSILLSEEVESGQAIVLQCSVKEGSGPITYKFYKEKENKPFHQVTLNDTQAIWHKPKASKDQEGQYYCLASNRATPSKNFLQSNILAVRVYLAPWKKGLIAVVVIAVIIAVLLLGARFYFLKKSKAKQMPVEMCRPAAPLLNSNNEKTLSDPNTEANRHYGYNEDVGNHAMKPLNENKEPLTLDVEYTEVEVTSPEPHRGLGTKGTETVYSEIRKADPDLVENRYSRTEGSLDGT

SEQ ID NO: 329 - protein, 739 aa, Bos taurus
  CD31
MQLRWTQRGMMWLGALLTLLLCSSLKGQENSFTINSIHMQILPHSTVQNGENLTLQCLVDVSTTSRVKPLHQVLFYKDDVLLHNVSSRRNTESYLIPHVRVCDSGRYKCNVILNNKEKTTPEYEVWVKGVSDPRVTLDKKEVIEGGVVVVNCSVPEEKAPVHFTIEKFELNIRGAKKKREKTSQNQNFVTLEFTVEEQDRTIRFQCQAKIFSGSNVESSRPIQSDLVTVRESFSNPKFHIIPEGKVMEGDDLQVKCTVQVTHQAQSFPEIIIQKDREIVAHNSLSSEAVYSVMATTEHNGNYTCKVEASRISKVSSVVVNVTELFSKPKLESSATHLDQGEDLNLLCSIPGAPPANFTIQKGGMTVSQTQNFTKRVSEWDSGLYTCVAGVGRVFKRSNTVQITVCEMLSKPSIFHDSRSEVIKGQTIEVSCQSVNGTAPIFYQLSNTSKPVANQSVGSNKPAIFRVKPTKDVEYCCSADNCHSHSKMFSEVLRVKVIAPVDEAQLVVLKGEVEPGEPIVFYCSVNEGSFPITYKFYKEKESKPFYQDTINATQIMWHKTTASKEYEGQYYCTASNRANLSKHVIQSNTLTVRVYLPLEKGLIAVVVIGVIIVTLVLGAKCYFLKKAKAKQMPVEMSRPAVPLLNSNNEKTLSDAGTEADRHYGYNEDVGNHAMKPLNENKEPLTLDVEYTEVEVTSPEPHQGLGTKGTETETVYSEIRKADPDFVENRYSRTEGSLDGS

SEQ ID NO: 330 - protein, 712 aa, Gallus gallus
  CD31
MYLALLVIFLQCSEICTQKRVFTFNAVHIEVQPSAKVKNGAPMSIICHADISKSTDFNLQHNITIFKDGKLVFMSVSGKGDARYEVPMARSSHTGDYQCAVEAGGKIKHSDSLHVWVTGMTKPILIAEKKEVFEGEVVKLRCELPEEVPPLYFVFQKMKVNSTPKQKLQEELYRNFSVVEFPVDEGDDILQFDCFGQRRVRFEFETSLTSNKTLITVKEPFIKPTLHIKPSSNITEGKEIRFECSTVVAKMRDIEIILLRNKTILSSVRDQTTLKYSAVATLEDSGEYLCKVEEGKASKTTTHNVVVTELFPKPILTASMNELDEHKELTLSCSIKDFQNASFSILRKHSSRDILLRNSRNLTMRASVNDTGSYICKAEVNRIVKESKPVTIRVYAPVSMPSISVFSGLPEVVLGRPLNLTCCLMMGTPPITFTFYKGNKNKKKIVTNNTCAAFLDKDIRQNDKGQYKCGAKNHHSSGMKTSKVLNITVIVPIRNTSLGSVPYGVVEDGRETTFLCSVEEGSWPIHFRIFRKMDRDILLFERSEHADRIVWQKKEMSRQDTGTYYCIASNRANVSAKSHPITINVILASWQKGVITAFVLIPIAGVMILAVWWFWCKKKNAKGPSMEMSRSALATNSTSEKLSRQHNDGDYYSGSGYIEDGENHMKSTDENKGPDLESAEVEYTEVEVSTLDPHRDSVENRHSRIQGRPDAT

SEQ ID NO: 331 - protein, 5 aa, Artificial Sequence
  peptide
NFVLM

SEQ ID NO: 332 - protein, 5 aa, Artificial Sequence
  peptide
NFVLI

SEQ ID NO: 333 - protein, 5 aa, Artificial Sequence
  peptide
NFMVM

SEQ ID NO: 334 - protein, 5 aa, Artificial Sequence
  peptide
NFMML

SEQ ID NO: 335 - protein, 5 aa, Artificial Sequence
  peptide
NFVTL

SEQ ID NO: 336 - protein, 5 aa, Artificial Sequence
  peptide
NSVTL

SEQ ID NO: 337 - protein, 5 aa, Artificial Sequence
  peptide
NFSVV

SEQ ID NO: 338 - protein, 14 aa, Artificial Sequence
  peptide
NFVILEFPVEEQDR

SEQ ID NO: 339 - protein, 16 aa, Artificial Sequence
  peptide
DQNFVILEFPVEEQDR

SEQ ID NO: 340 - protein, 22 aa, Artificial Sequence
  peptide
QENFVILEWLNKNCDEFFSLLL

SEQ ID NO: 341 - protein, 23 aa, Artificial Sequence
  peptide
DQENFVILEWLNKNCDEFFSLLL

SEQ ID NO: 342 - protein, 5 aa, Artificial Sequence
  SITE 1: Xaa is an amino acid residue with a hydrophobic side chain, prefe rably Xaa is selected from the group consisting of leucine (Leu, L), alanine (Ala, A), valine (Val, V), isoleucine (Ile, I), methi onine (Met, M), cysteine (Cys, C), phenylalanine (Phe, F), tyrosi
  peptide
  SITE 2: Xaa is an amino acid residue with a hydrophobic side chain or wit h a polar uncharged side chain, preferably Xaa is selected from t he group consisting of isoleucine (Ile, I), alanine (Ala, A), val ine (Val, V), leucine (Leu, L), methionine (Met, M), cysteine (Cy
  SITE 3: Xaa is any amino acid residue
  SITE 4: Xaa is an aromatic amino acid residue or an amino acid residue wi th a polar uncharged side chain, preferably Xaa is selected from the group consisting of phenylalanine (Phe, F), tyrosine (Tyr, Y) , tryptophan (Trp, W), serine (Ser, S), threonine (Thr, T), aspar
  SITE 5: Xaa is an amino acid residue with a polar uncharged side chain, preferably Xaa is selected from the group consisting of asparagine (Asn, N), glutamine (Gln, Q), serine (Ser, S), and threonine (Thr, T)
XXXXX

SEQ ID NO: 343 - protein, 198 aa, Artificial Sequence
  Domain III of human serum albumin
LVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVA

(19) United States
(12) Patent Application Publication
(10) Pub. No.: US 2023/0357354 A1
(43) Pub. Date: Nov. 9, 2023

(54) CD38-BINDING CD31 PEPTIDES AND USES THEREOF

(71) Applicant: ENCEFA, Saint-Maur-des-Fossés (FR)

(72) Inventors: Laurence BRESSAC, Saint-Maur-des-Fossés (FR); Serge GUERREIRO DA SILVA, Alfortville (FR); Damien TOULORGE, Tours (FR)

(73) Assignee: ENCEFA, Saint-Maur-des-Fossés (FR)

(21) Appl. No.: 18/006,246

(22) PCT Filed: Jul. 22, 2021
(86) PCT No.: PCT/EP2021/070602

§ 371 Date: Jan. 20, 2023

(30) Foreign Application Priority Data
Jul. 22, 2020 (EP) 20305843.3

(51) Int. Cl. C07K 14/705; A61K 47/64; A61P 3/10; A61P 13/12; A61P 3/04

(52) U.S. Cl. CPC C07K 14/70503; A61K 47/6425; A61P 3/04; A61P 3/10; A61P 13/12

(57) ABSTRACT

Peptides derived from CD31, specifically binding to CD38. Also, the use of these peptides in prevention and/or treatment of diseases, including diseases characterized by increased levels of soluble CD38 in a subject and/or a reduced soluble CD31 level/soluble CD38 level ratio, and reducing the severity of fibrosis and/or slowing down the progression of fibrosis and/or preventing the development of fibrosis in a subject. Further, methods of increasing the level of at least one anti-inflammatory cytokine in a subject, lowering glucose levels in a subject, and promoting or enhancing reparation of DNA lesions induced by a chemotherapeutic agent in a subject.

### FIELD OF INVENTION

The present invention relates to peptides derived from CD31, specifically binding to CD38; and their use in the prevention and/or treatment of diseases.

### BACKGROUND OF INVENTION

CD38 is a 45 kDa transmembrane protein that is ubiquitously expressed (Malavasi et al., 2008. Physiol Rev. 88(3):841-86). CD38 is mostly known for its enzymatic function that in part consists in degrading Nicotinamide Adenine Dinucleotide (NAD; Chini, 2009. Curr Pharm Des. 15(1):57-63), a metabolite involved in more than 400 redox reactions (Verdin, 2015. Science. 350(6265):1208-13). However, CD38 also possesses a receptor function whose physiological role remains elusive, that was serendipitously discovered in 1990 (Funaro et al., 1990. J Immunol. 145(8):2390-6) thanks to the use of antibodies that were later qualified as “agonistic”. The endogenous ligand activating CD38 receptor function, CD31, was only identified in 1998 (Deaglio et al., 1998. J Immunol. 160(1):395-402).

CD31, also known as Platelet endothelial cell adhesion molecule-1 (PECAM-1), is a 130 kDa type I transmembrane glycoprotein that consists of six extracellular immunoglobulins (Ig)-like homology domains, a 19-residue transmembrane domain, and a 118-residue cytoplasmic tail (Newman and Newman, 2003. Arterioscler Thromb Vasc Biol. 23:953-964). As such, CD31 belongs to the Ig superfamily of cell adhesion molecules. CD31 is able to bind to itself through homophilic interaction involving Ig-like domain 1; as well to two other ligands, αVβ3 integrin and CD38 (Newman, 1997. J Clin Invest. 99(1):3-8; Kalinowska & Losy, 2006. Eur J Neurol. 13(12):1284-90).

CD31 expression is mainly observed in endothelial cells, where it is considered as a constitutive marker (Kalinowska & Losy, 2006. Eur J Neurol. 13(12):1284-90), but also in most non-erythroid cells of the hematopoietic lineage including platelets, monocytes, neutrophils, T and B cell subsets (Wang et al., 2003. Am J Physiol Heart Circ Physiol. 284(3):H1008-17). While CD31 homophilic interaction is a major constituent of the endothelial cell intercellular junction, it is also involved in the process of leukocyteendothelial transmigration (diapedesis), allowing the penetration of leucocytes into tissue during inflammation (Ilan & Madri, 2003. Curr Opin Cell Biol. 15(5):515-24).

CD31 is found under both a membrane form and a soluble form, following metalloproteinase-dependent cleavage (Ilan et al., 2001. FASEB J. 15(2):362-72). Increased circulating levels of soluble CD31 (sCD31) were observed in some inflammatory diseases, including atherosclerosis and sepsis (Feng et al., 2016. Eur Rev Med Pharmacol Sci. 20(19):4082-4088; Kjaergaard et al., 2016. APMIS. 124(10):846-55), which might reflect the fact that sCD31 is involved in reducing further leukocyte transmigration in a negative feedback loop pattern (Muller et al., 1993. J Exp Med. 178(2):449-60).

As previously mentioned, the physiological role of the activation of the CD38 receptor function by CD31 remains elusive (Malavasi et al., 2008. Physiol Rev. 88(3):841-86). Here, the Inventors have described that activating this receptor function of CD38 using the extracellular domain of CD31 (sCD31) was able to protect neurons from cell death in vitro as well as in vivo, which was unexpected. The neuroprotective effect of sCD31 was antagonized in the presence of the neutralizing clone Moon-1 anti-CD31 antibody, as well as by antagonistic anti-CD38 antibodies (clone OKT10 or AT13/5), demonstrating that the neuroprotective effect of sCD31 is mediated through interaction with its ligand CD38. Moreover, the neuroprotective effect of sCD31 was recapitulated by agonistic anti-CD38 antibodies that were previously shown to mimic the interaction between sCD31 and CD38 (Deaglio et al., 1998. J Immunol. 160(1):395-402). Using a previously described agonistic anti-CD38 antibody (clone HB7) or proprietary agonistic anti-CD38 antibodies generated by phage display screening (WO 2020152290), the Inventors also observed that activation of CD38 receptor function had anti-inflammatory properties in vitro using human PBMCs as well as in vivo in a mouse model of ulcerative colitis. Altogether, these results demonstrate that engaging CD38 receptor function is of high therapeutic value.

How CD31 activates CD38 receptor function is still unknown. The only published data, dating back over 20 years ago, suggest that a fragment of CD31 consisting of the three first Ig-like domains of CD31 was still able to bind CD38 (Horenstein et al., 1998. Biochem J. 330(3):1129-35). For that reason, agonistic anti-CD38 antibodies are still the main option used to activate CD38 receptor function.

Here, the Inventors identified a minimal 5-amino acids peptide derived from human CD31, specifically binding to CD38, that activates CD38 receptor function. Based on the data disclosed herein, this CD31 peptide, and the peptides derived therefrom described herein, represent promising hits for the treatment of, inter alia, inflammatory diseases, autoimmune diseases, metabolic and endocrine diseases, fibrotic diseases, neuroinflammatory diseases and others.

### SUMMARY

The present invention relates to an isolated peptide which specifically binds to CD38, preferably human CD38, and which comprises an amino acid sequence X₁-X₂-X₃-X₄-X₅ with SEQ ID NO: 322, wherein:
X₁ is an amino acid residue with a polar uncharged side chain,
X₂ is an aromatic amino acid residue or an amino acid residue with a polar uncharged side chain,
X₃ is any amino acid residue,
X₄ is an amino acid residue with a hydrophobic side chain or with a polar uncharged side chain, and
X₅ is an amino acid residue with a hydrophobic side chain,
with the proviso that the isolated peptide does not comprise or consist of the full CD31^{Ig-} ^{like} ¹⁻² domains consisting of amino acid residues 35 to 233 of SEQ ID NO: 1, and
wherein said peptide does not consist of any one of SEQ ID NOs: 338, 339, 340 or 341.

In one embodiment,
X₁ is selected from the group consisting of asparagine (Asn, N), glutamine (Gln, Q), serine (Ser, S), and threonine (Thr, T),
X₂ is selected from the group consisting of phenylalanine (Phe, F), tyrosine (Tyr, Y), tryptophan (Trp, W), serine (Ser, S), threonine (Thr, T), asparagine (Asn, N), and glutamine (Gln, Q);
X₃ is any amino acid residue, preferably X3 is valine (Val, V),
X₄ is selected from the group consisting of isoleucine (Ile, I), alanine (Ala, A), valine (Val, V), leucine (Leu, L), methionine (Met, M), cysteine (Cys, C), phenylalanine (Phe, F), tyrosine (Tyr, Y), tryptophan (Trp, W), serine (Ser, S), threonine (Thr, T), asparagine (Asn, N), and glutamine (GIn, Q), and
X₅ is selected from the group consisting of leucine (Leu, L), alanine (Ala, A), valine (Val, V), isoleucine (Ile, I), methionine (Met, M), cysteine (Cys, C), phenylalanine (Phe, F), tyrosine (Tyr, Y), and tryptophan (Trp, W).

In one embodiment, the isolated peptide is linear. In one embodiment, the isolated peptide comprises an amino acid sequence selected from the group consisting of the sequences of Table 1.

In one embodiment, the isolated peptide is a 2-D structured peptide. In one embodiment, the 2-D structured peptide is cyclized and/or beta-turned. In one embodiment, the 2-D structured peptide is cyclized by disulfide bridge formation, head-to-tail cyclization, side-chain-to-side-chain cyclization, head-to-side-chain cyclization, side-chain-to-tail cyclization, thioether or ether bridge formation, lactone or thiolactone bridge formation, or lactam bridge formation. In one embodiment, the isolated peptide is cyclized and comprises an amino acid sequence selected from the group consisting of the sequences of Table 2. In one embodiment, the isolated peptide is beta-turned and comprises an amino acid sequence selected from the group consisting of the sequences of Table 3.

In one embodiment, the isolated peptide comprises an amino acid sequence selected from the group consisting of SEQ ID NOs: 6-10, 286-290, 295-303, 305, 306, 308, 311-313, 317, 320, 321, and 331-337.

In one embodiment, the isolated peptide comprises an amino acid sequence selected from the group consisting of SEQ ID NOs: 6-10, 286-290, 295, 296, 298-301, 305, 306, 308, 311-313, 317, 320, 321, and 331-337.

In one embodiment, the isolated peptide comprises an amino acid sequence selected from the group consisting of SEQ ID NOs: 6, 8-10, 286-290, 295, 298-301, 305, 306, 308, 312, and 320.

In one embodiment, the isolated peptide comprises at most 150 amino acid residues.

The present invention also relates to a conjugate comprising the peptide according to the present invention, fused to a payload being a therapeutic or diagnostic payload and/or a carrier payload.

The present invention also relates to a nucleic acid encoding the isolated peptide according to the preset invention or the conjugate according to the present invention.

The present invention also relates to an expression vector comprising the nucleic acid according to the present invention.

The present invention also relates to a composition comprising at least one isolated peptide according to the present invention or the conjugate according to the present invention.

The present invention also relates to the isolated peptide according to the present invention, or the conjugate according to the present invention, for use in preventing and/or treating a disease characterized by increased levels of soluble CD38 in a subject and/or a reduced soluble CD31 level/soluble CD38 level ratio.

The present invention also relates to the isolated peptide according to the present invention, or the conjugate according to the present invention, for use in preventing and/or treating a disease selected from inflammatory diseases; autoimmune diseases; metabolic and endocrine diseases; neurodegenerative diseases; neuroinflammatory diseases; cardiovascular diseases; kidney diseases; diabetic complications; ocular diseases; age-related diseases; fibrotic disorders; and cancer and metastasis; in a subject in need thereof.

In one embodiment, said disease is selected from diabetic complications; rheumatoid arthritis; systemic lupus erythematosus; diabetes; obesity; non-alcoholic steatohepatitis; chronic kidney disease; age-related macular degeneration; and glaucoma.

The present invention also relates to the isolated peptide according to the present invention, or the conjugate according to the present invention, for use in increasing the level of at least one anti-inflammatory cytokine in a subject in need thereof, in particular in the blood of said subject.

In one embodiment, said anti-inflammatory cytokine is interleukin-10 (IL-10).

The present invention also relates to the isolated peptide according to the present invention, or the conjugate according to the present invention, for use in lowering glucose levels in a subject in need thereof, in particular in the blood of said subject.

The present invention also relates to the isolated peptide according to the present invention, or the conjugate according to the present invention, for use in reducing the severity of fibrosis and/or slowing down the progression of fibrosis and/or preventing the development of fibrosis in a subject in need thereof.

The present invention also relates to the isolated peptide according to the present invention, or the conjugate according to the present invention, for use in promoting or enhancing reparation of DNA lesions induced by a chemotherapeutic agent in a subject in need thereof, wherein said chemotherapeutic agent is to be co-administered to said subject with the isolated peptide or conjugate, and/or wherein the conjugate’s payload is said chemotherapeutic agent.

### DEFINITIONS

Unless defined otherwise, all technical and scientific terms used herein have the same meaning as commonly understood by the skilled artisan. For convenience, the meaning of certain terms and phrases employed in the specification, examples and claims are provided.

### Amino Acid

Refers to organic compounds that contain amino (—NH₂) and carboxyl (COOH) functional groups, along with a side chain (R group) specific to each amino acid. The term “amino acid” encompasses “proteinogenic amino acids”, that is to say, the 22 amino acids that are genetically encoded and serve as natural building blocks of proteins; as well as “non-proteinogenic amino acids”, that is to say, those not naturally encoded or found in the genetic code of any organism.

Proteinogenic amino acids include: L-alanine (Ala, A), L-cysteine (Cys, C), L-aspartic acid (Asp, D), L-glutamic acid (Glu, E), L-phenylalanine (Phe, F), glycine (Gly, G), L-histidine (His, H), L-isoleucine (Ile, I), L-lysine (Lys, K), L-leucine (Leu, L), L-methionine (Met, M), L-asparagine (Asn, N), L-pyrrolysine (Pyl, O), L-proline (Pro, P), L-glutamine (Gln, Q), L-arginine (Arg, R), L-serine (Ser, S), L-threonine (Thr, T), L-selenocysteine (Sec, U), L-valine (Val, V), L-tryptophan (Trp, W), and L-tyrosine (Tyr, Y). Depending on the physicochemical properties of their side chain (R group), proteinogenic amino acids can be classified into:
hydrophobic amino acids, including alanine, isoleucine, leucine, methionine, cysteine, phenylalanine, tryptophane, tyrosine and valine;
branched-chain amino acids, including isoleucine, leucine and valine;
aromatic amino acids, including phenylalanine, tryptophan and tyrosine;
positively-charged amino acids, including arginine, histidine and lysine;
negatively-charged amino acids, including aspartic acid and glutamic acid;
uncharged polar amino acids, including asparagine, glutamine, serine and threonine;
sulfur-containing amino acids, including cysteine and methionine.

Examples of non-proteinogenic amino acids include, without limitation, p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, D-3-(2-naphthyl)alanine, 3-methyl-phenylalanine, O-4-allyl-L-tyrosine, 4-propyl-L-tyrosine, tri-O-acetyl-GlcNAc p-serine, L-Dopa, fluorinated phenylalanine, isopropyl-L-phenylalanine, p-azido-L-phenylalanine, p-acyl-L-phenylalanine, p-benzoyl-L-phenylalanine, 4-boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, azido-lysine, 6-azidonorleucine, 4-fluorophenylglycine, methyllysine, dimethyllysine, hydroxyproline, mercaptopropionic acid, 3-nitro-tyrosine, norleucine, pyro-glutamic acid, ε-acetyl-lysine, β-alanine, aminobutyric acid, citrulline, aminohexanoic acid, aminoisobutyric acid, cyclohexylalanine, nitro-arginine, nitro-phenylalanine, norvaline, octahydroindole carboxylate, ornithine, penicillamine, tetrahydroisoquinoline, 3-aminotyrosine, 3-nitrotyrosine, 3,4-dihydroxy-phenylalanine, 3-iodotyrosine, phenylselenocysteine, and dehydroalanine. Further examples of non-proteinogenic amino acids include, without limitation, acetyl-, acyl-, aldehyde-, alkenyl-, alkyl-, alkynyl-, amino-, aryl-, azido-, benzophenonyl-, benzoyl-, borate-, boronate-, cyano-, enone-, ester-, ether-, halo-, heterocyclic-, hydrazide-, hydrazine-, hydroxyl-, hydroxylamine-, imine-, iodide-, keto-, methoxy-, phosphine-, phospho-, phosphono-, seleno-, sulfonyl-, thioacid-, thiol-substituted amino acid residues, or any combination thereof. Finally, non-proteinogenic amino acids also include, without limitation, D-enantiomers of amino acid residues, whether proteinogenic or non-proteinogenic; in particular, D-enantiomers of amino acid residues include, without limitation, D-alanine, D-cysteine, D-aspartic acid, D-glutamic acid, D-phenylalanine, D-histidine, D-isoleucine, D-lysine, D-leucine, D-methionine, D-asparagine, D-pyrrolysine, D-proline, D-glutamine, D-arginine, D-serine, D-threonine, D-selenocysteine, D-valine, D-tryptophan, and D-tyrosine. Depending on the physicochemical properties of their side chain (R group), these non-proteinogenic amino acids can also be classified into the different groups detailed above.

### Binding Site, Binding Pocket

Refers to a specific arrangement of amino acids located on a protein (such as, e.g., on CD38) to which a compound (such as, e.g., the peptides according to the present invention) bind. Binding sites often consist of a chemically-active surface grouping of amino acids, and have specific 3-D structural characteristics as well as specific charge characteristics. Similarly to epitopes, binding sites can be linear or conformational, i.e., they can involve sequences of amino acids which are not necessarily contiguous in the primary structure of the protein.

### CD31

Refers to the 130 kDa type I transmembrane glycoprotein, also known as Platelet endothelial cell adhesion molecule-1 (PECAM-1), PECA1, GPIIA′, EndoCAM or CD31/EndoCAMas, and described in Newman & Newman (2003. Arterioscler Thromb Vasc Biol. 23:953-964). In the present invention, the term “CD31” refers more particularly to CD31 from a mammal species, even more particularly to human CD31. Preferably, the term “human CD31” refers to the protein of amino acid sequence SEQ ID NO: 1, referenced by the NP_000433 NCBI accession number (version 4 of Jun. 09, 2020). The numbering of amino acids of human CD31 as described herein corresponds to the numbering of amino acids of the human CD31 sequence set forth in SEQ ID NO: 1, and referenced by the NP_000433 NCBI accession number. Human CD31 is composed of several domains:
a signal peptide, from residues 1 to 27 of SEQ ID NO: 1;
an extracellular domain, from residues 28 to 601 of SEQ ID NO: 1 (herein referred to as “CD31^{extra}”), itself composed of:
   a first Ig-like C2-type domain (herein referred tas “CD31^{Ig-like} ¹”), from residues 35 to 121 of SEQ ID NO: 1,
   a second Ig-like C2-type domain (herein referred tas “CD31^{Ig-like} ²”), from residues 145 to 233 of SEQ ID NO: 1,
   a third Ig-like C2-type domain (herein referred tas “CD31^{Ig-like} ³”), from residues 236 to 315 of SEQ ID NO: 1,
   a fourth Ig-like C2-type domain (herein referred tas “CD31^{Ig-like} ⁴”), from residues 328 to 401 of SEQ ID NO: 1,
   a fifth Ig-like C2-type domain (herein referred tas “CD31^{Ig-like} ⁵”), from residues 424 to 493 of SEQ ID NO: 1,
   a sixth Ig-like C2-type domain (herein referred tas “CD31^{Ig-like} ⁶”), from residues 499 to 591 of SEQ ID NO: 1,
a transmembrane domain, from residues 602 to 620 of SEQ ID NO: 1; and
a cytoplasmic domain, from residues 621 to 738 of SEQ ID NO: 1.

Preferably, the term “soluble CD31” or “sCD31” refers to a soluble form of CD31 (i.e., not membrane-bound). Goldberger et al. (1994. J Biol Chem. 269(25):17183-17191) hypothesized that sCD31 was encoded by an alternatively spliced mRNA species from which the exon encoding the transmembrane domain had been remove. More recently, it was shown that the extracellular domain of CD31 comprising the first five Ig-like C2-type domains (CD31^{Ig-like} ¹ to CD31^{Ig-like} ⁵) was cleaved and shed from the surface of human T-cells upon activation via their TCR (Fornasa et al., 2010. J Immunol. 184(10):5485-5492). Accordingly, human sCD31 refers to the protein of amino acid sequence SEQ ID NO: 2, corresponding to amino acid residues 35 to 493 of SEQ ID NO: 1.

SEQ ID NO: 1MQPRWAQGATMWLGVLLTLLLCSSLEGQENSFTINSVD
MKSLPDWTVQNGKNLTLQCFADVSTTSHVKPQHQMLFYKDDVLFYNISSM
KSTESYFIPEVRIYDSGTYKCTVIVNNKEKTTAEYQVLVEGVPSPRVTLD
KKEAIQGGIVRVNCSVPEEKAPIHFTIEKLELNEKMVKLKREKNSRDQNF
VILEFPVEEQDRVLSFRCQARIISGIHMQTSESTKSELVTVTESFSTPKF
HISPTGMIMEGAQLHIKCTIQVTHLAQEFPEIIIQKDKAIVAHNRHGNKA
VYSVMAMVEHSGNYTCKVESSRISKVSSIVVNITELFSKPELESSFTHLD
QGERLNLSCSIPGAPPANFTIQKEDTIVSQTQDFTKIASKSDSGTYICTA
GIDKVVKKSNTVQIVVCEMLSQPRISYDAQFEVIKGQTIEVRCESISGTL
PISYQLLKTSKVLENSTKNSNDPAVFKDNPTEDVEYQCVADNCHSHAKML
SEVLRVKVIAPVDEVQISILSSKVVESGEDIVLQCAVNEGSGPITYKFYR
EKEGKPFYQMTSNATQAFWTKQKASKEQEGEYYCTAFNRANHASSVPRSK
ILTVRVILAPWKKGLIAVVIIGVIIALLIIAAKCYFLRKAKAKQMPVEMS
RPAVPLLNSNNEKMSDPNMEANSHYGHNDDVRNHAMKPINDNKEPLNSDV
QYTEVQVSSAESHKDLGKKDTETVYSEVRKAVPDAVESRYSRTEGSLDGT

SEQ ID NO: 2NSVDMKSLPDWTVQNGKNLTLQCFADVSTTSHVKPQHQ
MLFYKDDVLFYNISSMKSTESYFIPEVRIYDSGTYKCTVIVNNKEKTTAE
YQVLVEGVPSPRVTLDKKEAIQGGIVRVNCSVPEEKAPIHFTIEKLELNE
KMVKLKREKNSRDQNFVILEFPVEEQDRVLSFRCQARIISGIHMQTSEST
KSELVTVTESFSTPKFHISPTGMIMEGAQLHIKCTIQVTHLAQEFPEIII
QKDKAIVAHNRHGNKAVYSVMAMVEHSGNYTCKVESSRISKVSSIVVNIT
ELFSKPELESSFTHLDQGERLNLSCSIPGAPPANFTIQKEDTIVSQTQDF
TKIASKSDSGTYICTAGIDKVVKKSNTVQIVVCEMLSQPRISYDAQFEVI
KGQTIEVRCESISGTLPISYQLLKTSKVLENSTKNSNDPAVFKDNPTEDV
EYQCVADNCHSHAKMLSEVLR

### CD38

Refers to the 45 kDa type II transmembrane glycoprotein also known as T10, cyclic ADP-ribose hydrolase 1, ADPRC1, as described in Malavasi et al. (2008. Physiological Review. 88:841-886). In the present invention, the term “CD38” refers more particularly to particularly from a mammal species, even more particularly to human CD38. Preferably, the term “human CD38” refers to the protein of amino acid sequence SEQ ID NO: 3, referenced by the NP_001766 NCBI accession number (version 2 of May 24, 2020). The numbering of amino acids of human CD38 as described herein corresponds to the numbering of amino acids of the human CD38 sequence set forth in SEQ ID NO: 3, and referenced by the NP_001766 NCBI accession number.

SEQ ID NO: 3MANCEFSPVSGDKPCCRLSRRAQLCLGVSILVLILVVV
LAVVVPRWRQQWSGPGTTKRFPETVLARCVKYTEIHPEMRHVDCQSVWDA
FKGAFISKHPCNITEEDYQPLMKLGTQTVPCNKILLWSRIKDLAHQFTQV
QRDMFTLEDTLLGYLADDLTWCGEFNTSKINYQSCPDWRKDCSNNPVSVF
WKTVSRRFAEAACDVVHVMLNGSRSKIFDKNSTFGSVEVHNLQPEKVQTL
EAWVIHGGREDSRDLCQDPTIKELESIISKRNIQFSCKNIYRPDKFLQCV
KNPEDSSCTSEI

### Conjugate

With reference to a CD31 peptide, refers to a chimeric CD31 peptide which is bound to a payload, optionally through a linker, thereby forming a single molecule.

### Consist(s/ing) Essentially of

With reference to a composition, pharmaceutical composition or medicament, is used herein to intend that the compound according to the present invention is the only one agent with a biologic activity within said composition, pharmaceutical composition or medicament.

### Fragment

With reference to a CD31 peptide, refers to a portion of said CD31 peptide retaining the same or substantially the same biological function, activity and/or local structure, with respect to the specific biological function, activity and/or local structure identified for the CD31 peptide.

### Homolog

With reference to human CD31, refers to a distinct protein from another family or species which is determined by functional, structural or genomic analyses to correspond to human CD31. Most often, homologs will have functional, structural, or genomic similarities. Techniques are known by which homologs of a protein can readily be cloned using genetic probes and PCR. The identity of cloned sequences as homologous can be confirmed using functional assays and/or by genomic mapping of the genes.

### Medicament

Is meant to encompass a composition suitable for administration to a subject or patient, such as a mammal, especially a human. In general, a “medicament” is sterile and is usually free of contaminants that are capable of eliciting an undesirable response within the subject (e.g., the compound(s) in the medicament is pharmaceutical grade). Medicaments can be designed for administration to subjects in need thereof via a number of different routes of administration including oral, buccal, rectal, parenteral, intraperitoneal, intradermal, subcutaneous, intranasal, intrathecal, perispinal and the like. The preferred form depends on the intended mode of administration and therapeutic application. Typical preferred form are oral, injectable or infusible solutions.

### Peptide

Is intended to refer to a molecule that is at least composed of amino acids. A peptide can also possess other molecular groups like polysaccharide chains or other post-translational modifications. “Recombinant peptide” specifically refers to a peptide which is produced, expressed, generated or isolated by recombinant means, such as peptides which are expressed using a recombinant expression vector transfected into a host cell. Recombinant peptides include, for example, chimeric peptides. “Chimeric peptide” is intended to refer to a peptide that is created through the joining of two or more genes that originally coded for separate proteins or protein fragments; or through the fusion of two or more proteins or protein fragments. Chimeric peptides include, for example, a peptide fused to the Fc region of an IgG, a peptide fused to human serum albumin (HSA) or a particular domain (such as, e.g., domain III) of HSA, or to transferrin (as in Strohl, 2015. BioDrugs. 29(4): 215-239).

### Prevent, Preventing, Prevention

Refers to prophylactic and preventative measures, wherein the object is to reduce the chances that a subject will develop a given disease over a given period of time. Such a reduction may be reflected, e.g., in a delayed onset of at least one symptom of the disease in the subject.

### Subject

Refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human.

### Therapeutically Effective Amount

Means the level, amount or concentration of agent (e.g., a CD31 peptide specifically binding to CD38) that is aimed at, without causing significant negative or adverse side effects to the subject, (1) delaying or preventing the onset of the targeted disease(s); (2) slowing down or stopping the progression, aggravation, or deterioration of one or more symptoms of the targeted disease(s); (3) bringing about ameliorations of the symptoms of the targeted disease(s); (4) reducing the severity or incidence of the targeted disease(s); or (5) curing the targeted disease(s). A therapeutically effective amount may be administered prior to the onset of the targeted disease(s), for a prophylactic or preventive action. Alternatively or additionally, the therapeutically effective amount may be administered after initiation of the targeted disease(s), for a therapeutic action.

### Treat, Treating, Treatment, Alleviation

Refer to therapeutic treatment, excluding prophylactic or preventative measures; wherein the object is to slow down (lessen) a given disease. Those in need of treatment include those already with the disease as well those suspected to have the disease. A subject is successfully “treated” for a given disease if, after receiving a therapeutic amount of a peptide according to the present invention, said subject shows observable and/or measurable reduction in or absence of one or more of the following: one or more of the symptoms associated with the disease; reduced morbidity and mortality; and/or improvement in quality-of-life issues. The above parameters for assessing successful treatment and improvement in the targeted disease are readily measurable by routine procedures familiar to a physician.

### DETAILED DESCRIPTION

The present invention relates to peptides which specifically bind to CD38, preferably to human CD38.

In one embodiment, the peptides according to the present invention are CD31 peptides, preferably human CD31 peptides.

In one embodiment, the peptides according to the present invention are sCD31 peptides, preferably human sCD31 peptides.

In one embodiment, the peptides according to the present invention are isolated CD31 peptides, preferably isolated human CD31 peptides.

In one embodiment, the peptides according to the present invention are isolated sCD31 peptides, preferably isolated human sCD31 peptides.

In one embodiment, the peptides according to the present invention do not consist of the full-length CD31 protein, such as the full-length human CD31 protein.

In one embodiment, the peptides according to the present invention do not comprise or consist of the full CD31^{18-like} ¹⁻² domains comprising amino acid residues 35 to 233 of SEQ ID NO: 1.

In one embodiment, the peptides according to the present invention do not comprise or consist of the full CD31^{Ig-like} ² domain comprising amino acid residues 145 to 233 of SEQ ID NO: 1.

In one embodiment, the peptides according to the present invention do not consist of any one of SEQ ID NOs: 338, 339, 340 or 341.

In one embodiment, the peptides according to the present invention comprise an amino acid sequence X₁-X₂-X₃-X₄-X₅ with SEQ ID NO: 322, wherein:
X₁ is an amino acid residue with a polar uncharged side chain,
X₂ is an aromatic amino acid residue or an amino acid residue with a polar uncharged side chain,
X₃ is any amino acid residue,
X₄ is an amino acid residue with a hydrophobic side chain or with a polar uncharged side chain, and
X₅ is an amino acid residue with a hydrophobic side chain.

As used herein, the term “amino acid residue with a polar uncharged side chain” refers to amino acid residues which side chain (R group) is polar (i.e., having an electric dipole moment, with partial positive and negative charges) but is overall neutral in charge. These include the proteinogenic amino acid residues asparagine (Asn, N), glutamine (GIn, Q), serine (Ser, S) and threonine (Thr, T), as well as any non-proteinogenic amino acid residue that comprises such side chain.

As used herein, the term “aromatic amino acid residue” refers to amino acid residues which side chain (R group) comprises an aromatic ring. These include the proteinogenic amino acid residues phenylalanine (Phe, F), tyrosine (Tyr, Y) and tryptophan (Trp, W), as well as any non-proteinogenic amino acid residue that comprises such side chain.

By “any amino acid residue”, it is meant any of the 22 proteinogenic amino acid residues, as well as any non-proteinogenic amino acid residue.

As used herein, the term “amino acid residue with a hydrophobic side chain” refers to amino acid residues which side chain (R group) are mostly constituted of carbon and hydrogen atoms. They have a very small dipole moments, and tend to be repelled from water. Additionally or alternatively, “amino acid residue with a hydrophobic side chain” can be defined by their side chain hydrophobicity index (Δt_{R}) at pH = 7 equal or higher to 41 (Table 2 of Monera et al., 1995. J Pept Sci. 1(5):319-29). These include the proteinogenic amino acid residues isoleucine (Ile, I), alanine (Ala, A), valine (Val, V), leucine (Leu, L), methionine (Met, M), cysteine (Cys, C), phenylalanine (Phe, F), tyrosine (Tyr, Y), and tryptophan (Trp, W), as well as any non-proteinogenic amino acid residue that comprises such side chain.

In one embodiment, the peptides according to the present invention comprise an amino acid sequence X₁-X₂-X₃-X₄-X₅ with SEQ ID NO: 322, wherein:
X₁ is selected from the group comprising or consisting of asparagine (Asn, N), glutamine (Gln, Q), serine (Ser, S), and threonine (Thr, T),
X₂ is selected from the group comprising or consisting of phenylalanine (Phe, F), tyrosine (Tyr, Y), tryptophan (Trp, W), serine (Ser, S), threonine (Thr, T), asparagine (Asn, N), and glutamine (GIn, Q),
X₃ is any amino acid residue,
X₄ is selected from the group consisting of isoleucine (Ile, I), alanine (Ala, A), valine (Val, V), leucine (Leu, L), methionine (Met, M), cysteine (Cys, C), phenylalanine (Phe, F), tyrosine (Tyr, Y), tryptophan (Trp, W), serine (Ser, S), threonine (Thr, T), asparagine (Asn, N), and glutamine (GIn, Q), and
X₅ is selected from the group consisting of leucine (Leu, L), alanine (Ala, A), valine (Val, V), isoleucine (Ile, I), methionine (Met, M), cysteine (Cys, C), phenylalanine (Phe, F), tyrosine (Tyr, Y), and tryptophan (Trp, W).

In one embodiment, the peptides according to the present invention comprise an amino acid sequence NFVIL with SEQ ID NO: 321.

In one embodiment, the peptides according to the present invention are linear CD31 peptides. By linear peptide, it is meant a peptide which is devoid of secondary, let alone of tertiary structure.

In one embodiment, the peptides according to the present invention comprise or consist of the peptides of Table 1.

In one embodiment, the peptides according to the present invention are selected from the group comprising or consisting of the peptides of Table 1.

TABLE 1

linear CD31 peptides

| Amino acid sequence | SEQ ID NO: |
|---|---|
| NSRDQNFVILEFPVE | 6 |
| NFVILEF | 7 |
| NFVILEFP | 8 |
| KMVKLKREKNSRDQNFVILE | 70 |
| MVKLKREKNSRDQNFVILEF | 71 |
| KLKREKNSRDQNFVILEFPV | 72 |
| LKREKNSRDQNFVILEFPVE | 73 |
| KREKNSRDQNFVILE | 74 |
| REKNSRDQNFVILEF | 75 |
| EKNSRDQNFVILEFP | 76 |
| KNSRDQNFVILEFPV | 77 |
| RDQNFVILEFPVEEQ | 78 |
| DQNFVIL | 79 |
| DQNFVILEFPVEEQD | 80 |
| QNFVILE | 81 |
| QNFVILEFPVEEQDR | 82 |
| NFVILEFPVEEQDRV | 83 |
| RDQNFVILEFPVE | 138 |
| DQNFVILEFPVE | 139 |
| NFVILE | 140 |
| MVKLKREKNSRDQNFVILEFPVE | 151 |
| NSRDQNFVILEFPV | 286 |
| NSRDQNFVILEFP | 287 |
| NSRDQNFVILEF | 288 |
| NSRDQNFVILE | 289 |
| NSRDQNFVIL | 290 |
| NFAILEFP | 295 |
| NFVALEFP | 296 |
| NFVIAEFP | 297 |
| NFVILAFP | 298 |
| NFVILEAP | 299 |
| NFVILEFA | 300 |
| QFVILEFP | 301 |
| SFVILEFP | 302 |
| TFVILEFP | 303 |
| NYVILEFP | 305 |
| NWVILEFP | 306 |
| NSVILEFP | 308 |
| NFVFLEFP | 311 |
| NFVTLEFP | 312 |
| NFVQLEFP | 313 |
| NFVIFEFP | 317 |
| NSMLIEFT | 320 |
| NFVIL | 321 |
| NFVLM | 331 |
| NFVLI | 332 |
| NFMVM | 333 |
| NFMML | 334 |
| NFVTL | 335 |
| NSVTL | 336 |
| NFSVV | 337 |

In one embodiment, the peptides according to the present invention are 2-D structured CD31 peptides. By “2-D structured peptide”, it is meant a peptide which adopts a secondary structure in solution, such as, e.g., a single loop, double loop, triple loop, a β-sheet-like fold, an α-helix-like fold, etc. Such secondary structures can form naturally in solution and/or be constrained through the insertion of amino acid residues and/or chemical moieties triggering or stabilizing the formation of said secondary structures.

In one embodiment, the 2-D structured peptides according to the present invention are cyclized, i.e., they mimic a loop.

In one embodiment, cyclization to create a loop mimic may be carried out by any means known by the skilled artisan, including, without limitation, the formation of a disulphide bridge between cysteine residues; the formation of a lactam bridge between glutamic or aspartic acid and lysine residues; the formation of a lactone or thiolactone bridge between amino acid residues containing carboxyl, hydroxyl or mercapto functional groups; the formation of a thioether or ether bridge between amino acid residues containing hydroxyl or mercapto functional groups; the formation of an amide bond between the N-terminal α-amino group and the C-terminal α-carboxy group (i.e., head-to-tail cyclization); the formation of a bond between the side chains of two amino acid residues (i.e., side-chain-to-side-chain cyclization); the formation of a bond between the N-terminal α-amino group and the side chain of an amino acid residue (i.e., head-to-side-chain cyclization); the formation of a bond between the C-terminal α-carboxy group and the side chain of an amino acid residue (i.e., side-chain-to-tail cyclization); and the like.

In one embodiment, the reactive amino acid residues (e.g., the cysteine residues for the formation of a disulphide bridge, the glutamic or aspartic acid and lysine residues for the formation of a lactam bridge, etc.) may be already present in the peptide to be cyclized, or can alternatively be inserted at the N-terminal and/or C-terminal extremities of the peptide. In the latter case, it will be understood that reactive amino acid residue(s) present within the peptide sequence shall be either protected or mutated to another amino acid residue so as to avoid unwanted bridge formation.

In one embodiment, the 2-D structured peptides according to the present invention are peptides with SEQ ID NO: 322 as described above, wherein:
(i) a cysteine residue is inserted at both N-terminal and C-terminal extremities; and
(ii) any cysteine residue within the peptide sequence is either protected or mutated to another amino acid residue.

In one embodiment, the 2-D structured peptides according to the present invention are selected from the group comprising or consisting of the peptides of Table 1, wherein:
(i) a cysteine residue is inserted at both N-terminal and C-terminal extremities; and
(ii) any cysteine residue within the peptide sequence is either protected or mutated to another amino acid residue, preferably to serine.

In one embodiment, the 2-D structured peptides according to the present invention are selected from the group comprising or consisting of the peptides of Table 2.

TABLE 2

looped CD31 peptides

| Amino acid sequence | SEQ ID NO: |
|---|---|
| CRDQNFVILEFPVEEQC | 9 |
| CMVKLKREKNSRDQNFVILEFPVEC | 157 |
| CMVKLKREKNSRDQNFVILEFC | 163 |
| CNSRDQNFVILEFPVEC | 164 |
| CDQNFVILC | 195 |
| CQNFVILEC | 196 |
| CKREKNSRDONFVILEC | 247 |
| CREKNSRDONFVILEFC | 248 |
| CEKNSRDONFVILEFPC | 249 |
| CKNSRDQNFVILEFPVC | 250 |
| CDQNFVILEFPVEEQDC | 251 |
| CONFVILEFPVEEQDRC | 252 |
| CNFVILEFPVEEQDRVC | 253 |
| CNFVILEFC | 291 |

In one embodiment, the 2-D structured peptides according to the present invention are beta-turned, i.e., they mimic a β-sheet-like fold.

In one embodiment, constraining a peptide into a β-sheet-like fold may be carried out by any means known to the skilled artisan, including, without limitation, the insertion of a Pro-Gly dipeptide to induce a type II’ β-turn, the insertion of a Asp-Gly dipeptide to induce a type I′ β-turn, and the like.

Preferably, the dipeptide is inserted — or amino acid residues in the native peptide sequence are mutated into the desired dipeptide — at a position which is central in the peptide, i.e., the number of amino acid residues on one side of the dipeptide is not 50%, preferably 20%, more preferably 10% higher or lower than the number of amino acid residues on the other side of the dipeptide. The skilled artisan will readily understand that the dipeptide shall be at a position which is central in the peptide so as to allow a complete turn of the peptide to mimic a β-sheet-like fold.

It is readily understandable that the expression “the dipeptide is inserted” also encompasses cases where amino acid residues in the native peptide sequence are mutated into the desired dipeptide.

In one embodiment, the dipeptide is not inserted within the sequence of SEQ ID NO: 322 described above, but may be inserted N-terminally or C-terminally from this sequence with SEQ ID NO: 322. In this case, the overall peptide should be long enough to allow the dipeptide to remain somewhat central in the peptide.

In one embodiment, constraining a peptide into a β-sheet-like fold may further require cyclization of the peptide by any means known by the skilled artisan, including those described hereinabove.

In one embodiment, the 2-D structured peptides according to the present invention are peptides with SEQ ID NO: 322 as described above, wherein:
(i) a Pro-Gly or Asp-Gly dipeptide, preferably a Pro-Gly dipeptide, is inserted at a position which is central in the peptide; and optionally
(ii) a cysteine residue is inserted at both N-terminal and C-terminal extremities; and
(iii) any cysteine residue within the peptide sequence is either protected or mutated to another amino acid residue.

In one embodiment, the 2-D structured peptides according to the present invention are selected from the group comprising or consisting of the peptides of Table 1, wherein:
(i) a Pro-Gly or Asp-Gly dipeptide, preferably a Pro-Gly dipeptide, is inserted at a position which is central in the peptide; and optionally
(ii) a cysteine residue is inserted at both N-terminal and C-terminal extremities; and
(iii) any cysteine residue within the peptide sequence is either protected or mutated to another amino acid residue, preferably to serine.

In one embodiment, the 2-D structured peptides according to the present invention are selected from the group comprising or consisting of the peptides of Table 3.

TABLE 3

beta-turned CD31 peptides

| Amino acid sequence | SEQ ID NO: |
|---|---|
| CMVKLKREKNPGDQNFVILEFC | 10 |
| CKMVKLKREKPGRDQNFVILEC | 274 |
| CKLKREKNSRPGNFVILEFPVC | 275 |

When reciting “the peptides according to the present invention” or “the CD31 peptide(s)”, also encompassed are CD31 peptide variants, preferably human CD31 peptide variants.

CD31 peptide variants encompass (1) peptides of human CD31 homologs, (2) fragments of the CD31 peptides described above, (3) mutants of the CD31 peptides described above, (4) peptidomimetics of the CD31 peptides described above, and (5) conjugates of the CD31 peptides described above, including any combinations thereof; as long as said variant comprises an amino acid sequence X₁-X₂-X₃-X₄-X₅ with SEQ ID NO: 322 as defined above.

Homologs of human CD31 include, but are not limited to, CD31 from Mus musculus (SEQ ID NO: 323), from Rattus norvegicus (SEQ ID NO: 324), from Felis catus (SEQ ID NO: 325), from Canis lupus (SEQ ID NO: 326), from Ovis aries (SEQ ID NO: 327), from Sus scrofa (SEQ ID NO: 328), from Bos taurus (SEQ ID NO: 329), and from Gallus gallus (SEQ ID NO: 330). Hence, a CD31 peptideaccording to the present invention include peptides derived from CD31 homologs, as long as said peptide homolog comprises an amino acid sequence X₁-X₂-X₃-X₄-X₅ with SEQ ID NO: 322 as defined above.

In one embodiment, the peptides according to the present invention are selected from the group comprising or consisting of SEQ ID NOs: 331 to 337, all of which are derived from CD31 homologs.

In one embodiment, a fragment of a peptide as described herein above comprises at least 70%, preferably at least 75%, 80%, 85%, 90%, 95%, 96%, 97%, 98%, 99% or more amino acids of said peptide; as long as said peptide fragment comprises an amino acid sequence X₁-X₂-X₃-X₄-X₅ with SEQ ID NO: 322 as defined above.

In one embodiment, a mutant of a peptide as described herein above shares at least 70%, preferably at least 75%, 80%, 85%, 90%, 95%, 96%, 97%, 98%, 99% or more sequence identity, preferably local sequence identity with said peptide; as long as said peptide mutant comprises an amino acid sequence X₁-X₂-X₃-X₄-X₅ with SEQ ID NO: 322 as defined above.

Sequence identity refers to the number of identical or similar amino acids in a comparison between a test and a reference sequence. Sequence identity can be determined by sequence alignment of protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps as

$$\frac{n u m b e r o f i d e n t i c a l p o s i t i o n s}{l e n g t h o f t h e t o t a l a l i g n e d s e q u e n c e} \times 100.$$

A global alignment is an alignment that aligns two sequences from beginning to end, aligning each letter in each sequence only once. An alignment is produced, regardless of whether or not there is similarity or identity between the sequences. For example, 50% sequence identity based on global alignment means that in an alignment of the full sequence of two compared sequences, each of 100 nucleotides in length, 50% of the residues are the same. It is understood that global alignment can also be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman & Wunsch, 1970. J Mol Biol. 48(3):443-53). Exemplary programs and software for performing global alignment are publicly available and include the Global Sequence Alignment Tool available at the National Center for Biotechnology Information (NCBI) website (http://ncbi.nlm.nih.gov), and the program available at deepc2.psi.iastate.edu/aat/align/align.html.

A local alignment is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Smith & Waterman, 1981. Adv Appl Math. 2(4):482-9). For example, 50% sequence identity based on local alignment means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include:
(1) a unary comparison matrix (containing a value of 1 for identities and 0 for non-identities) and the weighted comparison matrix of Gribskov & Burgess (1986. Nucleic Acids Res. 14(16):6745-63), as described by Schwartz & Dayhoff (1979. Matrices for detecting distant relationships. In Dayhoff (Ed.), Atlas of protein sequences. 5:353-358. Washington, DC: National Biomedical Research Foundation);
(2) a penalty of 3.0 for each gap and an additional 0.10 penalty for each symbol in each gap; and
(3) no penalty for end gaps.

Whether any peptide and peptide mutant have amino acid sequences that are at least 70%, preferably at least 75%, 80%, 85%, 90%, 95%, 96%, 97%, 98%, 99% or more “identical”, or other similar variations reciting a percent identity, can be determined using known computer algorithms based on local or global alignment (see, e.g., https://en.wikipedia.org/wiki/List_of_sequence_alignment_software [last accessed on July 2021, 2020], providing links to dozens of known and publicly available alignment databases and programs).

Generally, for purposes herein, sequence identity is determined using computer algorithms based on global alignment, such as the Needleman-Wunsch Global Sequence Alignment tool available from NCBI/BLAST (http://blast.ncbi.nlm.nih.gov/Blast.cgi); or LAlign (William Pearson implementing the Huang and Miller algorithm [Huang & Miller, 1991. Adv Appl Math. 12(3):337-57).

Typically, the full-length sequence of each of the compared peptides is aligned across the full-length of each sequence in a global alignment. Local alignment also can be used when the sequences being compared are substantially the same length.

Therefore, the term identity represents a comparison or alignment between a test and a reference peptide. In one exemplary embodiment, “at least 70% of sequence identity” refers to percent identities from 70 to 100% relative to the reference peptide. Identity at a level of 70% or more is indicative of the fact that, assuming for exemplification purposes a test and reference peptide length of 100 amino acids are compared, no more than 30 out of 100 amino acids in the test peptide differ from those of the reference peptide. Such differences can be represented as point mutations randomly distributed over the entire length of an amino acid sequence or they can be clustered in one or more locations of varying length up to the maximum allowable, e.g., 30/100 amino acid difference (approximately 70% identity). Differences can also be due to deletions or truncations of amino acid residues. Differences are defined as amino acid substitutions, insertions or deletions. Depending on the length of the compared sequences, at the level of homologies or identities above about 85-90%, the result can be independent of the program and gap parameters set; such high levels of identity can be assessed readily, often without relying on software.

Peptidomimetics of the peptides described hereinabove comprise, but are not limited to, retro analogues, inverso analogues and retroinverso analogues of said peptides.

The term “retro analogue” with reference to a peptide means a reversed sequence of amino acid residues of said peptide, i.e., an amino acid sequence X₅-X₄-X₃-X₂-X₁ with SEQ ID NO: 342, with X₁, X₂, X₃, X₄ and X₅ being as defined above; the term “inverso analogue” with reference to a peptide means the same sequence of amino acid residues of said peptide but with D-amino acid residues instead of L-amino acid residues. The term “retroinverso analogue” with reference to a peptide encompasses both a reversed sequence of amino acid residues of said peptide (i.e., an amino acid sequence X₅-X₄-X₃-X₂-X₁ with SEQ ID NO: 342), with D-amino acid residues instead of L-amino acid residues.

In one embodiment, the peptidomimetics is a retro analogue. In one embodiment, the peptidomimetics is an inverso analogue. In one embodiment, the peptidomimetics is a retroinverso analogue.

In one embodiment, a conjugate of a peptide according to the present invention comprises or consists of said peptide fused to a payload.

In one embodiment, the payload may be a therapeutic or diagnostic payload, i.e., a fused therapeutic or diagnostic molecule which role is to support, supplement, synergize and/or target the therapeutic or diagnostic role of the peptide according to the present invention.

Examples of therapeutic or diagnostic payloads suitable for conjugation with peptides according to the present invention include, but are not limited to, peptides, polypeptides, proteins, polymers, nucleic acid molecules, small molecules, mimetic agents, synthetic drugs, inorganic molecules, organic molecules, radioisotopes, nanoparticles, vectors, and the like.

Alternatively, or additionally, examples of therapeutic or diagnostic payloads suitable for conjugation with peptides according to the present invention include, but are not limited to, chemotherapeutic agents, targeted therapy agents, cytotoxic agents, antibiotics, antivirals, cell cycle-synchronizing agents, ligands for cellular receptor(s), immunomodulatory agents, pro-apoptotic agents, anti-angiogenic agents, cytokines, growth factors, antibodies or antigen-binding fragments thereof, hormones, coding or non-coding oligonucleotides, photodetectable labels, contrast agents, radiolabels, and the like.

It will be apparent that some therapeutic or diagnostic payloads may fall into more than one category. It will also be readily understood that the conjugation of a peptide according to the present invention with a particular therapeutic or diagnostic payload, which may be chosen among those recited herein but without being limited thereto, depends on the intended use of said conjugate, e.g., on a specific disease or condition to be prevented and/or treated.

In one embodiment, the therapeutic or diagnostic payload is a chemotherapeutic agent.

As used herein, the term “chemotherapeutic agent” refers to any molecule that is effective in inhibiting tumor growth.

Suitable examples of chemotherapeutic agents include, but are not limited to:
alkylating agents, such as, e.g.:
   nitrogen mustards, including chlormethine, cyclophosphamide, ifosfamide, trofosfamide, chlorambucil, melphalan, prednimustine, bendamustine, uramustine, chlornaphazine, cholophosphamide, estrarnustine, mechlorethamine, mechlorethamine oxide hydrochloride, novembichin, phenesterine, uracil mustard and the like;
   nitrosoureas, including carmustine, lomustine, semustine, fotemustine, nimustine, ranimustine, streptozocin, chlorozotocin, and the like;
   alkyl sulfonates, including busulfan, mannosulfan, treosulfan, and the like;
   aziridines, including carboquone, thiotepa, triaziquone, triethylenermelamine, benzodopa, meturedopa, uredopa, and the like; hydrazines, including procarbazine, and the like;
   triazenes, including dacarbazine, temozolomide, and the like; ethylenimines and methylamelamines, including altretamine, triethylenemelamine, trietylenephosphoramide, triethylenethiophosphaorarnide, trimethylolomelamine and the like;
   and others, including mitobronitol, pipobroman, actinomycin, bleomycin, mitomycins (including mitomycin C, and the like), plicamycin, and the like;
acetogenins, such as, e.g., bullatacin, bullatacinone, and the like;
benzodiazepines, such as, e.g., 2-oxoquazepam, 3-hydroxyphenazepam, bromazepam, camazepam, carburazepam, chlordiazepoxide, cinazepam, cinolazepam, clonazepam, cloniprazepam, clorazepate, cyprazepam, delorazepam, demoxepam, desmethylflunitrazepam, devazepide, diazepam, diclazepam, difludiazepam, doxefazepam, elfazepam, ethyl carfluzepate, ethyl dirazepate, ethyl loflazepate, flubromazepam, fletazepam, fludiazepam, flunitrazepam, flurazepam, flutemazepam, flutoprazepam, fosazepam, gidazepam, halazepam, iclazepam, irazepine, kenazepine, ketazolam, lorazepam, lormetazepam, lufuradom, meclonazepam, medazepam, menitrazeparn, metaclazepam, motrazepam, N-desalkylflurazepam, nifoxipam, nimetazepam, nitemazepam, nitrazepam, nitrazepate, noniazepam, nortetrazepam, oxazepam, phenazepam, pinazepam, pivoxazepam, prazepam, proflazepam, quazepam, QH-II-66, reclazepam, R04491533, Ro5-4864, SH-I-048A, sulazepam, temazepam, tetrazepam, tifluadom, tolufazepam, triflunordazepam, tuclazepam, uldazepam, arfendazam, clobazam, CP-1414S, lofendazam, triflubazam, girisopam, GYKI-52466, GYKI-52895, nerisopam, talampanel, tofisopam, adinazolam, alprazolam, bromazolam, clonazolam, estazolam., flualprazolam, flubromazolam, flunitrazolam, nitrazolam, pyrazolam, triazolam, bretazenil, climazolam, EVT-201, FG-8205, flumazenil, GL-II-73, imidazenil, ¹²³I-iomazenil, L-655,708, loprazolam, midazolam, PWZ-029, remimazolam, Ro15-4513, Ro48-6791, Ro48-8684, Ro4938581, sarmazenil, SH-053-R-CH3-2′F, cloxazolam, flutazolam, haloxazolam, mexazolam, oxazolam, bentazepam, clotiazepam, brotizolam, ciclotizolam, deschloroetizolam, etizolam, fluclotizolam, israpafant, JQ1, metizolam, olanzapine, telenzepine, lopirazepam, zapizolam, razobazam, ripazepam, zolazepam, zomebazam, zometapine, premazepam, clazolam, anthramycin, avizafone, rilmazafone, and the like;
antimetabolites, such as, e.g.:
   antifolates, including aminopterin, methotrexate, pemetrexed, pralatrexate, pteropterin, raltitrexed, denopterin, trimetrexate, pemetrexed, and the like;
   purine analogues, including pentostatin, cladribine, clofarabine, fludarabine, nelarabine, tioguanine, mercaptopurine, and the like;
   pyrimidine analogues, including fluorouracil, capecitabine, doxifluridine, tegafur, tegafur/gimeracil/oteracil, carmofur, floxuridine, cytarabine, gemcitabine, azacytidine, decitabine, and the like; and
   hydroxycarbamide;
androgens, such as, e.g., calusterone, dromostanolone propionate, epitiostanol, mepitiostane, testolactone, and the like;
anti-adrenals, such as, e.g., aminoglutethimide, mitotane, trilostane, and the like;
folic acid replenishers, such as, e.g., frolinic acid, and the like;
maytansinoids, such as, e.g., maytansine, ansamitocins, and the like;
platinum analogs, such as, e.g., platinum, carboplatin, cisplatin, dicycloplatin, nedaplatin, oxaliplatin, satraplatin, and the like;
antihormonal agents, such as, e.g.:
   anti-estrogens, including tamoxifen, raloxifene, aromatase inhibiting 4(5)-imidazoles, 4..hydroxytamoxifen, trioxifene, keoxifene, LY117018, onapristone, toremifene, and the like;
   anti-androgens, including flutamide, nilutamide., bicalutamide, leuprolide, goserelin, and the like;
trichothecenes, such as, e.g., T-2 toxin, verracurin A, roridinA, anguidine and the like;
toxoids, such as, e.g., cabazitaxel, docetaxel, larotaxel, ortataxel, paclitaxel, tesetaxel, and the like;
others, such as, e.g., camptothecin (including the synthetic analogue topotecan); bryostatin; callystatin; CC-1065 (including its adozelesin, carzelesin and bizelesin synthetic analogues); cryptophycins (including cryptophycin 1 and cryptophycin 8); dolastatin; duocarmycin (including its synthetic analogues KW-2189 and CBI-TMI); eleutherobin; pancratistatin; sarcodictyin; spongistatin; aclacinomysins; authramycin; azaserine; bleomycin; cactinomycin; carabicin; canninomycin: carzinophilin; chromomycins; dactinomycin; daunorubicin; detorubicin; 6-diazo-5-oxo-L-norleucine; doxorubicin (including morpholino-doxorubicin, cyanomorpholino-doxorubicin, 2-pyrrolino-doxorubicin, deoxydoxorubicin, and the like); epirubicin; esorubicin; idanrbicin; marcellomycin; mycophenolic acid; nogalarnycin; olivomycins; peplomycin; potfiromycin; puromycin; quelamycin; rodorubicin; streptomgrin; streptozocin; tubercidin; ubenimex; zinostatin; zorubicin; aceglatone; aldophospharnide glycoside; aminolevulinic acid; amsacrine; bestrabucil; bisantrene; edatraxate; defofamine; demecolcine; diaziquone; elfornithine; elliptinium acetate; epothilone; etoglucid; gallium nitrate; hydroxyurea; lentinan; lonidamine; mitoguazone; mitoxantrone; mopidamol; nitracrine; phenamet; pirarubicin: podophyllinic acid; 2-ethylhydrazide; PSK®; razoxane; rhizoxin; sizofiran; spirogennanium; tenuazonic acid; 2,2′,2″-trichlorotriethylarnine; urethan; vindesine; dacarbazine; mannomustine; mitobromtol; mitolactol; pipobroman; gacytosine; arabinoside; 6-thioguanine; vinblastine; etoposide; vincristine; vinorelbine; navelbine; novantrone; teniposide; daunomycin; xeloda; ibandronate; CPT-11; topoisomerase inhibitor RFS 2000; topoisomerase I inhibitor SN38; difluoromethylomithine; retinoic acid; and the like.

In one embodiment, the therapeutic or diagnostic payload is a targeted therapy agent.

As used herein, the term “targeted therapy agent” refers to any molecule which aims at one or more particular target molecules (such as, e.g., proteins) involved in tumor genesis, tumor progression, tumor metastasis, tumor cell proliferation, cell repair, and the like.

Suitable examples of targeted therapy agents include, but are not limited to, tyrosine-kinase inhibitors, serine/threonine kinase inhibitors, monoclonal antibodies and the like.

Suitable examples of targeted therapy agents include, but are not limited to, HER1/EGFR inhibitors (such as, e.g., brigatinib, erlotinib, gefitinib, olmutinib, osimertinib, rociletinib, vandetanib, and the like); HER2/neu inhibitors (such as, e.g., afatinib, lapatinib, neratinib, and the like); C-kit and PDGFR inhibitors (such as, e.g., axitinib, masitinib, pazopanib, sunitinib, sorafenib, toceranib, and the like); FLT3 inhibitors (such as, e.g., lestaurtinib, and the like); VEGFR inhibitors (such as, e.g., axitinib, cediranib, lenvatinib, nintedanib, pazopanib, regorafenib, semaxanib, sorafenib, sunitinib, tivozanib, toceranib, vandetanib, and the like); RET inhibitors (such as, e.g., vandetanib, entrectinib, and the like); c-MET inhibitors (such as, e.g., cabozantinib, and the like); bcr-abl inhibitors (such as, e.g., imatinib, dasatinib, nilotinib, ponatinib, radotinib, and the like); Src inhibitors (such as, e.g., bosutinib, dasatinib, and the like); Janus kinase inhibitors (such as, e.g., lestaurtinib, momelotinib, ruxolitinib, pacritinib, and the like); MAP2K inhibitors (such as, e.g., cobimetinib, selumetinib, trametinib, binimetinib, and the like); EML4-ALK inhibitors (such as, e.g., alectinib, brigatinib, ceritinib, crizotinib, and the like); Bruton’s inhibitors (such as, e.g., ibrutinib, and the like); mTOR inhibitors (such as, e.g., everolimus, temsirolimus, and the like); hedgehog inhibitors (such as, e.g., sonidegib, vismodegib, and the like); CDK inhibitors (such as, e.g., palbociclib, ribociclib, and the like); anti-HER1/EGFR monoclonal antibodies (such as, e.g., cetuximab, necitumumab, panitumumab, and the like); anti-HER2/neu monoclonal antibodies (such as, e.g., ado-trastuzumab emtansine, pertuzumab, trastuzumab, trastuzumab-dkst, and the like); anti-EpCAM monoclonal antibodies (such as, e.g., catumaxomab, edrecolomab, and the like); anti-VEGF monoclonal antibodies (such as, e.g., bevacizumab, bevacizumab-awwb, and the like); anti-CD20 monoclonal antibodies (such as, e.g., ibritumomab, obinutuzumab, ocrelizumab, ofatumumab, rituximab, tositumomab, and the like); anti-CD30 monoclonal antibodies (such as, e.g., brentuximab, and the like); anti-CD33 monoclonal antibodies (such as, e.g., gemtuzumab, and the like); and anti-CD52 monoclonal antibodies (such as, e.g., alemtuzumab, and the like).

In one embodiment, the therapeutic or diagnostic payload is a cytotoxic agent.

As used herein, the term “cytotoxic agent” refers to any molecule that results in cell death by any mechanism.

Suitable examples of cytotoxic agents include, but are not limited to, anthracyclines, vinca alkaloids, steroids, and chemotherapeutic agents.

Suitable examples of anthracyclines include, but are not limited to, aclarubicin, amrubicin, daunorubicin, doxorubicin, epirubicin, idarubicin, pirarubicin, valrubicin and zorubicin.

Suitable examples of vinca alkaloids include, but are not limited to, vinblastine, vincristine, vinflunine, vindesine and vinorelbine.

Suitable examples of steroids include, but are not limited to, estrogen receptor modulators, androgen receptor modulators and progesterone receptor modulators.

Suitable examples of chemotherapeutic agents have been described hereinabove.

In one embodiment, the therapeutic or diagnostic payload is an antibiotic.

Suitable examples of antibiotics include, but are not limited to:
aminoglycosides, such as, e.g., amikacin, gentamicin, kanamycin, neomycin, netilmicin, streptomycin, tobramycin, paromycin, and the like;
ansamycins, such as, e.g., geldanamycin, herbimycin and the like;
carbacephems, such as, e.g., loracarbef and the like;
carbapenems, such as, e.g., ertapenum, doripenem, imipenem, cilastatin, meropenem, and the like;
first generation cephalosporins, such as, e.g., cefadroxil, cefazolin, cefalotin, cephalexin, and the like;
second generation cephalosporins, such as, e.g., ceflaclor, cefamandole, cefoxitin, cefprozil, cefuroxime, and the like;
third generation cephalosporins, such as, e.g., cefixime, cefdinir, cefditoren, cefoperazone, cefotaxime, cefpodoxime, ceftazidime, ceftibuten, ceftizoxime, ceftriaxone, and the like;
fourth generation cephalosporins, such as, e.g., cefepime and the like;
fifth generation cephalosporins, such as, e.g., ceftobiprole, and the like;
glycopeptides, such as, e.g., teicoplanin, vancomycin, and the like;
macrolides, such as, e.g., axithromycin, clarithromycin, dirithromycine, erythromycin, roxithromycin, troleandomycin, telithromycin, spectinomycin, and the like;
monobactams, such as, e.g., axtreonam, and the like;
penicilins, such as, e.g., amoxicillin, ampicillin, axlocillin, carbenicillin, cloxacillin, dicloxacillin, flucloxacillin, mezlocillin, meticillin, nafcilin, oxacillin, penicillin, peperacillin, ticarcillin, and the like;
antibiotic polypeptides, such as, e.g., bacitracin, colistin, polymyxin B, and the like;
quinolones, such as, e.g., ciprofloxacin, enoxacin, gatifloxacin, levofloxacin, lemefloxacin, moxifloxacin, norfloxacin, orfloxacin, trovafloxacin, and the like;
sulfonamides, such as, e.g., mafenide, prontosil, sulfacetamide, sulfamethizole, sulfanilamide, sulfasalazine, sulfisoxazole, trimethoprim, trimethoprim-sulfamethoxazole, and the like;
tetracyclines, such as, e.g., demeclocycline, doxycycline, minocycline, oxytetracycline, tetracycline, and the like; and
others such as, e.g., arspenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin/rifampicin, tinidazole, and the like.

In one embodiment, the therapeutic or diagnostic payload is an antiviral

Suitable examples of antivirals include, but are not limited to, acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, zinc salts, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscarnet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

In one embodiment, the therapeutic or diagnostic payload is a cell cycle-synchronizing agent.

As used herein, the term “cell cycle-synchronizing agent” refers to any molecule able to for unify the cell cycle of a population of cells to the same phase upon administration.

Suitable examples of cell cycle-synchronizing agents include, but are not limited to, aphidicolin, butyrolactone I, colchicine, cycloheximide, demecolcine, dimethyl sulfoxide, 5-fluorodeoxyuridine, Hoechst 33342, mimosine, nocodazole, roscovitine, and thymidine.

In one embodiment, the therapeutic or diagnostic payload is a ligand for a cellular receptor.

As used herein, the term “ligand for a cellular receptor” refers to any molecule binding to a cellular receptor (such as a cell surface receptor, an intracellular receptor or a co-receptor, including transcription factors and the like), including agonists and antagonists, as well as partial agonists, inverse agonists, and allosteric modulators.

Suitable examples of ligands for cellular receptors include, but are not limited to, ligands binding to the AATYK receptors, the acetylcholine receptors, the ADGRG receptors, the adiponectin receptors, the adrenergic α1 receptors, the adrenergic α2 receptors, the adrenergic β1 receptors, the adrenergic β2 receptors, the adrenergic β3 receptors, the adrenomedullin receptor, the AMPA receptors, the anaphylatoxin receptors, the angiopoietin receptors, the angiotensin receptors, the anti-Müllerian hormone receptor, the apelin receptor, the asialoglycoprotein receptors, the AXL receptors, the benzodiazepine receptor, the bile acid receptor, the bombesin receptors, the bone morphogenetic protein receptors, the bradykinin receptors, the brain-specific angiogenesis inhibitors, the cadherin receptors, the calcitonin receptor, the calcitonin receptor-like receptor, the calcium-sensing receptor, the cannabinoid receptors, the CD97 receptor, the chemokine receptors, the cholecystokinin receptors, the complement receptors, the corticotropin-releasing hormone receptors, the CysLT receptors, the cytokine receptors, the DDR receptors, the dopamine receptors, the EBI2 receptor, the ectodysplasin A receptor, the EGF module-containing mucin-like hormone receptors, the EGF receptors, the endothelin receptors, the EPH receptors, the estrogen receptor, the FGF receptors, the free fatty acid receptors, the frizzled receptors, the FSH receptor, the GABAB receptors, the galanin receptors, the GHB receptor, the ghrelin receptor, the glucagon receptors, the glucagon-like peptide receptors, the glutamate receptors, the glycine receptors, the gonadotropin receptors, the gonadotropin-releasing hormone receptors, the GPRC6A receptor, the growth factor receptors, the growth hormone receptors, the growth-hormone-releasing hormone receptor, the guanylate cyclase-coupled receptors, the HGF receptors, the histamine receptors, the hydroxycarboxylic acids receptors, the immunoglobulin immune receptors, the insulin receptors, the kainate receptors, the KiSS1-derived peptide receptor, the latrophilin receptors, the leptin receptor, the leukotriene B4 receptors, the lipoprotein receptor-related protein receptors, the LTK receptors, the luteinizing hormone/choriogonadotropin receptor, the lysophosphatidic acid receptors, the lysophospholipid receptors, the mannose receptor, the MAS receptors, the melanin-concentrating hormone receptors, the melanocortin receptors, the melatonin receptors, the methuselah-like proteins receptors, the motilin receptor, the MuSK receptors, the N-acetylglucosamine receptor, the neuromedin receptors, the neuropeptide B/W receptors, the neuropeptide FF receptors, the neuropeptide S receptor, the neuropeptide Y receptors, the neuropilins receptor, the neurotensin receptors, the N- formyl peptide receptor, the nicotinic acetylcholine receptors, the NMDA receptors, the nuclear receptors, the olfactory receptor, the opioid receptors, the opsin receptors, the orexin receptors, the oxoeicosanoid receptor, the oxoglutarate receptor, the oxytocin receptor, the parathyroid hormone receptors, the PDGF receptors, the pituitary adenylate cyclase-activating polypeptide type I receptor, the platelet-activating factor receptor, the progestin and adipoQ receptors, the prokineticin receptors, the prolactin receptor, the prolactin-releasing peptide receptor, the prostacyclin receptor, the prostaglandin receptors, the protease-activated receptor, the PTK7 receptors, the purinergic adenosine receptors, the purinergic P2X receptors, the purinergic P2Y receptors, the relaxin receptors, the RET receptors, the retinoic acid-inducible orphan G-protein-coupled receptors, the ROR receptors, the ROS receptors, the RYK receptors, the scavenger receptors, the secretin receptor, the serine/threonine-specific protein kinase receptors, the serotonine receptors, the smoothened receptor, the somatostatin receptors, the sphingosine-1-phosphate receptors, the SREB receptors, the stimulator of interferon genes (STING) receptor, the succinate receptor, the tachykinin receptors, the thromboxane receptor, the thyrotropin receptor, the thyrotropin-releasing hormone receptor, the toll-like receptors, the trace-amine associated receptors, the transferrin receptor, the Trk receptors, the tumor necrosis factor receptors, the tyrosine phosphatase receptors, the urotensin-II receptor, the vasoactive intestinal peptide receptors, the vasoactive intestine peptide receptors, the vasopressin receptors, the VEGF receptors, the vomeronasal receptor, and the zinc-activated ion channel receptor.

In one embodiment, the therapeutic or diagnostic payload is an immunomodulatory agent.

Suitable examples of immunomodulatory agents include, but are not limited to, immunostimulatory agents and immunosuppressor agents.

Suitable examples of immunostimulatory agents include, but are not limited to, cytokines (such as, e.g., filgrastim, pegfilgrastim, lenograstim, molgramostim sargramostim, ancestim, albinterferon, interferon alfa natural, interferon alfa 2a, peginterferon alfa-2a, interferon alfa 2b, peginterferon alfa-2b, interferon alfa n1, interferon alfacon-1, interferon alpha-n3, interferon beta natural, interferon beta 1a, interferon beta 1b, interferon gamma, aldesleukin, oprelvekin, and the like); immune checkpoint inhibitors (such as, e.g., inhibitors of CTLA4, PD-1, PD-L1, LAG-3, B7-H3, B7-H4, TIM3, A2AR, and/or IDO, including nivolumab, pembrolizumab, pidilizumab, AMP-224, MPDL3280A, MDX-1105, MEDI-4736, arelumab, ipilimumab, tremelimumab, pidilizumab, IMP321, MGA271, BMS-986016, lirilumab, urelumab, PF-05082566, IPH2101, MEDI-6469, CP-870,893, mogamulizumab, varlilumab, avelumab, galiximab, AMP-514, AUNP 12, indoximod, NLG-919, INCB024360, and the like); toll-like receptor agonists (such as, e.g., buprenorphine, carbamazepine, ethanol, fentanyl, GS-9620, imiqimod, lefitolimod, levorphanol, methadone, morphine, (+)-morphine, morphine-3-glucuronide, oxcarbazepine, oxycodone, pethidine, resiquimod, SD-101, tapentadol, tilsotolimod, VTX-2337, glucuronoxylomannan from Cryptococcus, MALP-2 from Mycoplasma, MALP-404 from Mycoplasma, OspA from Borrelia, porin from Neisseria or Haemophilus, hsp60, hemmaglutinin, LcrV from Yersinia, bacterial flagellin, lipopolysaccharide, lipoteichoic acid, lipomannan from Mycobacterium, glycosylphosphatidylinositol, lysophosphatidylserine, lipophosphoglycan from Leishmania, zymosan from Saccharomyces, Pam2CGDPKHPKSF, Pam3CSK4, CpG oligodeoxynucleotides, poly(I:C) nucleic acid sequences, poly(A:U) nucleic acid sequences, double-stranded viral RNA, and the like); STING receptor agonists (such as, e.g., those described in WO2017/100305, vadimezan, CL656, ADU-S100, 3′3′-cGAMP, 2′3′-cGAMP, ML RR-S2 CDG, ML RR-S2 cGAMP, cyclic di-GMP, DMXAA, DiABZI, and the like); CD1 ligands; growth hormone; immunocyanin; pegademase; prolactin; tasonermin; female sex steroids; histamine dihydrochloride; poly ICLC; vitamin D; lentinan; plerixafor; roquinimex; mifamurtide; glatiramer acetate; thymopentin; thymosin al; thymulin; polyinosinic:polycytidylic acid; pidotimod; Bacillus Calmette-Guérin; melanoma vaccine; sipuleucel-T; and the like.

Suitable examples of immunosuppressor agents include, but are not limited to:
antimetabolites, such as, e.g.:
   antifolates, including aminopterin, methotrexate, pemetrexed, pralatrexate, pteropterin, raltitrexed, denopterin, trimetrexate, pemetrexed, and the like;
   purine analogues, including pentostatin, cladribine, clofarabine, fludarabine, nelarabine, tioguanine, mercaptopurine, and the like;
   pyrimidine analogues, including fluorourail, capecitabine, doxifluridine, tegafur, tegafur/gimeracil/oteracil, carmofur, floxuridine, cytarabine, gemcitabine, azacytidine, decitabine, and the like; and
   hydroxycarbamide);
macrolides, such as, e.g., tacrolimus, ciclosporin, pimecrolimus, abetimus, gusperimus, and the like;
immunomodulatory imide drugs, such as, e.g., lenalidomide, pomalidomide, thalidomide, apremilast, and the like;
1L-1 receptor antagonists, such as, e.g., anakinra, and the like);
mTOR inhibitors, such as, e.g., sirolimus, everolimus, ridaforolimus, temsirolimus, umirolimus, zotarolimus, and the like);
serum-targeting antibodies, such as, e.g., eculizumab, adalimumab, afelimomab, certolizumab pegol, golimumab, infliximab, nerelimomab, mepolizumab, omalizumab, faralimomab, elsilimomab, lebrikizumab, ustekinumab, secukinumab, and the like;
cell-targeting antibodies, such as, e.g., muromonab-CD3, otelixizumab, teplizumab, visilizumab, clenoliximab, keliximab, zanolimumab, efalizumab, erlizumab, obinutuzumab, rituximab, ocrelizumab, pascolizurnab, gomiliximab, lumiliximab, teneliximab, toralizumab, aselizumab, galiximab, gavilimomab, ruplizumab, belimumab, blisibimod, ipilimumab, tremelimumab, bertilimumab, lerdelimumab, metelimumab, natalizumab, tocilizumab, odulimomab, basiliximab, daclizumab, inolimomab, zolimomab aritox, atorolimumab, cedelizumab, fontolizumab, maslimomab, morolimumab, pexelizumab, reslizumab, rovelizumab, siplizumab, talizumab, telimomab aritox, vapaliximab, vepalimomab, and the like;
fusion antibodies, such as, e.g., abatacept, belatacept, etanercept, pegsunercept, aflibercept, alefacept, rilonacept and the like.

In one embodiment, the therapeutic or diagnostic payload is a pro-apoptotic agent.

As used herein, the term “pro-apoptotic agent” refers to any molecule able to induce apoptosis or programmed cell death in a cell upon administration.

Suitable examples of pro-apoptotic agents include, but are not limited to, histone deacetylase inhibitors (such as, e.g., sodium butyrate, depsipeptide and the like), bortezomib, deguelin, favopiridol, fenretinide, fludarabine, kaempferol, miltefosine, narciclasine, obatoclax, oblimersen, and oncrasin.

In one embodiment, the therapeutic or diagnostic payload is an anti-angiogenic agent.

As used herein, the term “anti-angiogenic agent” refers to a molecule that reduces or prevents angiogenesis, which is responsible for the growth and development of blood vessels.

Suitable examples of anti-angiogenic agents include, but are not limited to, inhibitors of any of the vascular endothelial growth factor VEGF-A, VEGF-B, VEGF-C, or VEGF-D, which are major inducers of angiogenesis in normal and pathological conditions, and are essential in embryonic vasculogenesis.

Additionally or alternatively, an anti-angiogenic agent also can inhibit other angiogenic factors, such as, without limitation, a member of the fibroblast growth factor (FGF) family such as FGF-1 (acidic), FGF-2 (basic), FGF-4 or FGF-5; or angiopoietin-1, a factor that signals through the endothelial cell-specific Tie2 receptor tyrosine kinase; or the receptor of any of these angiogenic factors.

In one embodiment, the therapeutic or diagnostic payload is a cytokine.

Suitable examples of cytokines include, but are not limited to, chemokines, tumor necrosis factors, interleukins, and colony-stimulating factors.

Suitable examples of chemokines include, but are not limited to, chemokine C-C motif ligand (CCL) 1, CCL2, CCL3, CCL4, CCL5, CCL6, CCL7, CCL8, CCL9, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, chemokine C-X-C motif ligand (CXCL) 1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, fractalkine, chemokine C motif ligand (XCL) 1, and XCL2.

Suitable examples of tumor necrosis factors include, but are not limited to, tumor necrosis factor (TNF) α, lymphotoxin, OX40L, CD40LG, Fas ligand, CD70, CD153, 4-1BB ligand, TNF-related apoptosis-inducing ligand (TRAIL), receptor activator of nuclear factor κ-B ligand (RANKL), a proliferation-inducing ligand (APRIL), B-cell activating factor (BAFF), and ectodysplasin A (EDA).

Suitable examples of interleukins include, but are not limited to, interleukin- (IL-) 1α, IL-1β, IL-1Ra, IL-2, IL-3, IL-4, IL-5, IL-6, IL-7, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36α, IL-36β, IL-36γ, IL-36Ra, IL-37, IL-38, interferon (IFN) α, IFNβ, IFNκ, and IFNω.

Suitable examples of colony-stimulating factors include, but are not limited to, granulocyte-macrophage colony-stimulating factor (GM-CSF) (including granulocyte-colony stimulating factor (G-CSF) and macrophage colony-stimulating factor (M-CSF)), haematopoietin, and thrombopoietin.

In one embodiment, the therapeutic or diagnostic payload is a growth factor.

Suitable examples of growth factors include, but are not limited to, fibroblast growth factor (FGF) 1, FGF2, FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF23, transforming growth factor (TGF) α, epidermal growth factor (EGF), heparin-binding EGF-like growth factor (HB-EGF), transforming growth factor (TGF) β, insulin-like growth factor (IGF) 1, IGF2, Platelet-derived growth factor (PDGF) subunit A (PDGFA), PDGF subunit B (PDGFB), PDGF subunit C (PDGFC), PDGF subunit D (PDGFD), vascular endothelial growth factor (VEGF)-A, VEGF-B, VEGF-C, VEGF-D, placental growth factor (PGF), nerve growth factor (NGF), and hepatocyte growth factor (HGF).

In one embodiment, the therapeutic or diagnostic payload is an antibody or an antigen-binding fragment thereof.

Suitable examples of antibodies or antigen-binding fragments thereof include, but are not limited to, monoclonal antibodies, polyclonal antibodies, bispecific antibodies, multispecific antibodies, antibody fragments, and antibody mimetics, such as, e.g., scFv, di-scFv, tri-scFv, single domain antibodies, nanobodies, bispecific T-cell engagers (BiTEs), Fab, F(ab′)2, Fab′, chemically linked Fab, X-Link Fab, tandem-scFv/BiTE, diabodies, tandem diabodies, diabody-Fc fusions, tandem diabody-Fc fusion, tandem diabody-CH3 fusion, tetra scFv-Fc fusion, dual variable domain immunoglobulin, knob·hole, strand exchange engineered domain, CrossMab, quadroma-derived bispecific antibody, single domain based antibody, affibodies, affilins, affimers, affitins, alphabodies, anticalins, avimer, DARPins, Kunitz domain peptides, monobodies and nanoCLAMPs.

In one embodiment, the therapeutic or diagnostic payload is a hormone.

Suitable examples of hormones include, but are not limited to, GnRH, TRH, dopamine, CRH, GHRH, somatostatin, MCH, oxytocin, vasopressin, FSH, LH, TSH, prolactin, POMC, CLIP, ACTH, MSH, endorphins, lipotropin, GH, aldosterone, cortisol, cortisone, DHEA, DHEA-S, androstenedione, epinephrine, norepinephrine, thyroid hormone T3, thyroid hormone T4, calcitonin, PTH, testosterone, AMH, inhibin, estradiol, progesterone, activin, relaxin, GnSAF, hCG, HPL, estrogen, glucagon, insulin, amylin, pancreatic polypeptide, melatonin, N,N-dimethyltryptamine, 5-methoxy-N,N-dimethyltryptamine, thymosin α1, beta thymosins, thymopoietin, thymulin, gastrin, ghrelin, CCK, GIP, GLP-1, secretin, motilin, VIP, enteroglucagon, peptide YY, IGF-1, IGF-2, leptin, adiponectin, resistin, osteocalcin, renin, EPO, calcitriol, prostaglandin, ANP, and BNP.

In one embodiment, the therapeutic or diagnostic payload is a coding or non-coding oligonucleotide.

Suitable examples of coding or non-coding oligonucleotides include, but are not limited to, messenger RNA (mRNA), antisense RNA (asRNA), small interfering RNA (siRNA), microRNA (miRNA), anti-miRNA (antimiR), long non-coding RNA (IncRNA) (such as, e.g., transfer RNA [tRNA], ribosomal RNA [rRNA], and the like), small temporal RNA (stRNA), trans-acting siRNA, short hairpin RNA (shRNA), cis-natural antisense transcripts (NATs), CRISPR RNA, long noncoding RNA, piwi-interacting RNA (piRNA), repeat-associated siRNA (rasiRNA), RNA aptamers, ribozymes, and the like.

Further suitable examples of coding or non-coding oligonucleotides include, but are not limited to, recapuldencel-T, TriMix, BI-1361849, nusinersen, volanesorsen sodium, eteplirsen, ATL1105, ASM-8, inclisiran, patisiran, RXI-109, fitusiran, cemdisiran, QPI-1002, BMS-986263, PF-655, pegaptanib, avacincaptad pegol sodium, olaptesed pegol, emapticap pegol, SPC3649, bevasiranib, AGN-745, QPI-1007, TD101, SYL040012, SYL1001, Excellair, ALN-RSV01, CEQ508, siG12D LODER, TKM-ApoB, TKM-PLK1, ALN-VSP02, ALN-TTR01, Ber-Abl siRNA, Atu027, 15NP, CALAA-01, FANG vaccine, iPsiRNA, Tat/Rev shRNA, ARC1779, ARC19499, AS1411 (AGRO001), Fovista, NOX-A12, NOX-E36, NOX-H94, NU172, RB006 plus RB007, ARC1905, as well as those described in Table 1 and Table 2 of Crooke et al., 2018 (Cell Metab. 27(4):714-739), herein incorporated by reference.

In one embodiment, the therapeutic or diagnostic payload is a photodetectable label.

As used herein, the terms “photodetectable label” or “fluorophore” refer to a molecule that can re-emit light upon light excitation.

Suitable examples of photodetectable labels include, but are not limited to, Alexa Fluor® dyes, BODIPY® dyes, fluorescein, 5-carboxyfluorescein, 5-(4,6-dichlorotriazin-2-yl) aminofluorescein, 2′7′-dimethoxy-4′5′-dichloro-6-carboxyfluorescein, fluorescein isothiocyanate (FITC), QFITC, Oregon Green® 488, Oregon Green® 514, rhodamine and derivatives thereof (such as, e.g., rhodamine green, rhodamine green-X, rhodamine red-X, X-rhodamine., 6-carboxy-X-rhodamine (ROX), 6-carboxyrh(xiamine. (R6G), N,N,N′,N′-tetramethyl-6-carboxyrhodamine (TAMRA), lissamine rhodamine B, rhodamine 123, rhodamine X isothiocyanate, sulforhodamine B, sulforhodamine 101 (Texas Red), tetramethyl rhodamine, tetramethyl rhodamine isothiocyanate (TRITC)), eosin, eosin isothiocyanate, erythrosine, erythrosine B, erythrosin isothiocyanate, Texas Red®, Texas Red®X, naphthofluorescein, malachite green, malachite green isothiocyanate, coumarin derivatives, Pacific Orange, cascade blue, cascade yellow, dansyl chloride, dapoxyl dye, 1-dimethylamine-N(2-azido-ethyl)naphthalene-5-sulfonamide, 6-(6-amino-2-(2-azidoethyl)-1,3-dioxo-1H- benzo(de)-2(3H)isoquinoline, 6-(6-amino-2-(2-propinyl)-1,3-dioxo-1H-benzo(de)-2(3H)isoquinoline, 8-(4-azidoethyloxyphenyl)-2,6-diethyl-1,3,5,7-tetramethyl-4,4-difluoro-4-bora-3a,4a-diaza-s-indacene, 8-(4-propynyioxyphenyl)-2,6-diethyl-1,3,5,7-tetramethyl-4,4-difluoro-4-bora-3a,4a-diaza-s-indacene, 1-(3-azido-propoxy)-7-methylamino-phenoxazin-3-one, 1-(2-propynyl)-7-methylamino-phenoxazm-3-one, N-(5-(3-azidopropylamino)-9H-benzo(a)-phenoxa-2-in-9-ylidene)-N-methyl-methanaminium chloride, N-(5-(3-propynyl-amino)-9H-benzo(a)-phenoxazin-9-ylene)-N-methyl-methanaminium chloride, (9-(3-azido-propoxy)-7-piperidin-1-yl-phenoxazin-3-ylidene)-dimethyl-ammonium perchlorate, 4-acetamido-4′-isothiocyanatostilbene-2,2′-disulfonic acid, acridine, acridine isothiocyanate, 5-(2′-aminoethyl)aminonaphthalene-1-sulfonic acid, 4-amino-N-[3-vinylsulfonyl)phenyl]naphthalimide-3,5 disulfonate, N-(4-anilino-1-naphthyl)maleimide, anthranilamide, Brilliant Yellow, coumarin, coumarin derivatives, 7-amino-4-methylcoumarin, 7-amino-trifluoromethylcouluarin, cyanosine, 4′,6-diaminidino-2-phenylindole, 5′,5″-dibromopyrogallol-sulfonephthalein, 7-diethylamino-3-(4′-isothiocyanatophenyl)-4-methylcoumarin-4,4′-diisothiocyanatodihydro-stilbene-2,2′-disulfonic acid, 4,4′-diisothiocyanatostilbene-2,2′-disulfonic acid, ethidium, IR144, IR1446, 4-methylumbelliferone, o-cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4, riboflavin, rosolic acid, lanthanide chelates, quantum dots, cyanines, pyrelium dyes, and squaraines.

In one embodiment, the therapeutic or diagnostic payload is a contrast agent.

As used herein, the term “contrast agent” refers to any molecule used to increase the contrast of structures or fluids within the body in medical imaging. Contrast agents absorb or alter external electromagnetism or ultrasound (which differs from radiolabels which emit radiation themselves).

Suitable examples of contrast agents include, but are not limited to, diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, calcium iopodate, iopydol, propyliodone, iofendylate, lipiodol, barium sulfate, gadobenic acid, gadobutrol, gadodiamide, gadofosveset, gadolinium, gadopentetic acid, gadoteric acid, gadoteridol, gadoversetamide, gadoxetic acid, ferric ammonium citrate, mangafodipir, ferumoxsil, ferristene, perflubron, microspheres of human albumin, microparticles of galactose, perflenapent, microspheres of phospholipids, sulfur hexafluoride, and the like.

In one embodiment, the therapeutic or diagnostic payload is a radiolabel.

As used herein, the terms “radiolabel” or “radiopharmaceutical” refer to any molecule which emits radiation. Radiolabels can be used for therapeutics or diagnostic purposes.

Suitable examples of radiolabels include, but are not limited to, ^{99m}Tc compounds (such as, e.g., exametazime, medronic acid, macroaggregated albumin, sestamibi, tetrofosmin, exametazime, sulesomab, tilmanocept, arcitumomab, votumumab, hynic-octreotide, and the like); ¹²³I, ¹²⁵I or ¹³¹I compounds (such as, e.g., ioflupane, iofetamine, iomazenil, sodium iodohippurate, iobenguane, iodocholesterol, minretumomab, tositumomab, and the like); ¹⁸F compounds (such as, e.g., florbetapir, flutemetamol, fluciclovine, fludeoxyglucose, fluoroethyltyrosine, sodium fluoride, and the like); ⁶⁴Cu compounds (such as, e.g., Cu-ETS2, and the like); ⁷⁵Se compounds (such as, e.g., SeHCAT); ¹¹¹In compounds (such as, e.g., imciromab, capromab pendetide, satumomab pendetide, and the like); ⁸²Rb compounds (such as, e.g., rubidium chloride); ¹⁵³Sm compounds (such as, e.g., lexidronam, and the like); ⁸⁹Sr compounds (such as, e.g., strontium-89 chloride, and the like); ⁹⁰Y compounds (such as, e.g., ibritumomab tiuxetan, and the like); ²²³Ra compounds (such as, e.g., radium-223 chloride, and the like); ¹⁷⁷Lu compounds (such as, e.g., oxodotreotide, and the like); and any compounds comprising at least one ²H, ³H, ¹¹C, ¹³N, ¹⁴C, ¹⁵O, ¹⁸F, ²²Na, ²⁴Na, ³²P, ⁴⁷Ca, ⁵¹Cr, ⁵⁷Co, ⁵⁸Co, ⁵⁹Fe, ⁶⁴Cu, ⁶⁷Ga, ⁶⁸Ga, ⁷⁵Se, ⁸¹mKr, ⁸²Rb, ⁸⁹Sr, ⁹⁰Y, ^{99m}Tc, ¹¹¹In, ¹²³I, ¹²⁵I, ¹³¹I, ¹³³Xe, ¹⁵³Sm, ¹⁶⁵Dy, ¹⁶⁹Er, ¹⁷⁷Lu ¹⁸⁶Re, ¹⁹⁸Au, ²⁰¹T1 and/or ²²³Ra atom.

Additionally or alternatively, the payload may be a carrier, i.e., a fused moiety which role is to improve the pharmacokinetics and/or biodistribution of the peptide according to the present invention. Such conjugates are sometimes referred to as “biobetters”. For a review, see, e.g., Strohl, 2015. BioDrugs. 29(4):215-239.

In one embodiment, the carrier is chosen in the group comprising or consisting of a Fc domain of an immunoglobulin, transferrin or a domain thereof, albumin or a domain thereof, an XTEN sequence, a homo-amino acid polymer (HAP), a proline-alanine-serine polymer (PAS), an elastin-like peptide (ELP), polyethylene glycol, and hyaluronic acid.

In one embodiment, the carrier is chosen in the group comprising or consisting of a Fc domain of an immunoglobulin, transferrin or a domain thereof, and albumin or a domain thereof.

In one embodiment, the carrier is a domain of human serum albumin, such as, e.g., domain III of human serum albumin. Domain III of human serum albumin comprises or consists of amino acid sequence SEQ ID NO: 343.

In one embodiment where the carrier is a Fc domain of an immunoglobulin, the peptide conjugate does not mediate, trigger or enhance antibody-dependent cellular cytotoxicity (ADCC), antibody-dependent cellular phagocytosis (ADCP) and/or complement-dependent cytotoxicity (CDC).

In one embodiment, the carrier is a fatty acid that binds to human serum albumin.

Exemplary fatty acids that bind to human serum albumin include, but are not limited to, saturated or unsaturated fatty acid with aliphatic tails typically comprising from 12 to 40 carbons, preferably from 12 to 30 carbons, more preferably from 12 to 25 carbon atoms. These include, without limitation, lauric acid (C12:0), lauroleic acid (C12:1), laurolinoleic acid (C12:2), tridecylic acid (C13:0), myristic acid (C14:0), myristovaccenic acid (C14:1), myristoleic acid (C14:1), 8-tetradecenoic acid (C14:1), myristolinoleic acid (C14:2), myristolinolenic acid (C14:3), pentadecylic acid (C15:0), palmitic acid (C16:0), palmitovaccenic acid (C16:1), palmitoleic acid (C16:1), sapienic acid (C16:1), 4-hexadecenoic acid (C16:1), palmitolinolenic acid (C16:3), palmitidonic acid (C16:4), margaric acid (C17:0), stearic acid (C18:0), 12-octadecenoic acid (C18:1), vaccenic acid (C18:1), oleic acid (C18:1), elaidic acid (trans-C18:1), petroselinic acid (C18:1), linoleic acid (C18:2), linolelaidic acid (trans-C18:2), rumenic acid (C18:2), α-linolenic acid (C18:3), α-eleostearic acid (C18:3), β-eleostearic acid (trans-C18:3), punicic acid (C18:3), 7,10,13-octadecatrienoic acid (C18:3), γ-linolenic acid (C18:3), calendic acid (C18:3), pinolenic acid (C18:3), stearidonic acid (C18:4), nonadecylic acid (C19:0), arachidic acid (C20:0), paullinic acid (C20:1), gondoic acid (C20:1), gadoleic acid (C20:1), 8-eicosenoic acid (C20:1), dihomo-linoleic acid (C20:2), 8,11-eicosadienoic acid (C20:2), dihomo-α-linolenic acid (C20:3), 9,12,15-eicosatrienoic acid (C20:3), dihomo-γ-linolenic acid (C20:3), 7,10,13-eicosatrienoic acid (C20:3), mead acid (C20:3), eicosatetraenoic acid (C20:4), β-eicosatetraenoic acid (C20:4), arachidonic acid (C20:4), eicosapentaenoic acid (C20:5), heneicosylic acid (C21:0), behenic acid (C22:0), erucic acid (C22:1), adrenic acid (C22:4), clupanodonic acid (C22:5), osbond acid (C22:5), docosahexaenoic acid (C22:6), tricosylic acid (C23:0), lignoceric acid (C24:0), nervonic acid (C24:1), 9,12,15,18,21-tetracosapentaenoic acid (C24:5), 6,9,12,15,18,21-tetracosahexaenoic acid (C24:6), pentacosylic acid (C25:0), cerotic acid (C26:0), carboceric acid (C27:0), montanic acid (C28:0), nonacosylic acid (C29:0), melissic acid (C30:0), hentriacontylic acid (C31:0), lacceroic acid (C32:0), psyllic acid (C33:0), geddic acid (C34:0), ceroplastic acid (C35:0), hexatriacontylic acid (C36:0), heptatriacontylic acid (C37:0), octatriacontylic acid (C38:0), nonatriacontylic acid (C39:0), and tetracontylic acid (C40:0). It will be appreciated that fatty acid chain length may be selected by the skilled artisan on the basis of the intended use of the product and required circulating half-life.

In one embodiment, the carrier is an adsorption enhancer. In one embodiment, the carrier increases transcellular stomach adsorption.

Exemplary adsorption enhancers include, without limitation, those described in §0053-0171 of WO2015/127416A1, the content of which paragraphs is incorporated herein by this reference.

In one embodiment, the carrier comprises or consists of sodium N-[8-(2-hydroxybenzoyl)amino]caprylate (SNAC).

The specific binding between the peptides according to the present invention and CD38 implies that said peptides exhibit appreciable affinity for CD38.

The affinity of the peptides according to the present invention for CD38 can be determined by various methods well known to the skilled artisan. These methods can be qualitative or semi-quantitative (such as, e.g., co-immunoprecipitation, pull-down assay, and the like), or can be quantitative (such as, e.g., isothermal titration calorimetry, surface plasmon resonance, biolayer interferometry, and the like).

Alternatively or additionally, whether the peptides according to the present invention bind to CD38 can be tested readily by, inter alia, comparing the reaction of said peptides with CD38 or a fragment thereof (in particular, a fragment comprising or consisting of the binding site) with the reaction of said peptides with proteins or antigens other than CD38 or a fragment thereof.

In one embodiment, the peptides according to the present invention bind specifically to CD38 with a dissociation constant (K_{d}) lower than about 1×10⁻⁶ M, such as, lower than about 9×10⁻⁷ M, 8×10⁻⁷ M, 7×10⁻⁷ M, 6×10⁻⁷ M, 5×10⁻⁷ M, 4×10⁻⁷ M, 3×10⁻⁷ M, 2×10⁻⁷ M, 1×10⁻⁷ M, 9×10⁻⁸ M, 8×10⁻⁸ M, 7×10⁻⁸ M, 6×10⁻⁸ M, 5×10⁻⁸ M, 4×10⁻⁸ M, 3×10⁻⁸ M, 2×10⁻⁸ M, 1×10⁻⁸ M, 9×10⁻⁹ M, 8×10⁻⁹ M, 7×10⁻⁹ M, 6×10⁻⁹ M, 5×10⁻⁹ M, 4×10⁻⁹ M, 3×10⁻⁹ M, 2×10⁻⁹ M, 1×10⁻⁹ M, 9×10⁻¹⁰ M, 8×10⁻¹⁰ M, 7×10⁻¹⁰ M, 6×10⁻¹ M, 5×10⁻¹⁰ M, 4×10⁻¹⁰ M, 3×10⁻¹⁰ M, 2×10⁻¹⁰ M, 1×10⁻¹⁰ M or less.

In one embodiment, the peptides according to the present invention bind specifically to CD38 with a dissociation constant (K_{d}) in the nanomolar range or lower, such as about 1×10⁻ ⁷ M, 9×10⁻⁸ M, 8×10⁻⁸ M, 7×10⁻⁸ M, 6×10⁻⁸ M, 5×10⁻⁸ M, 4×10⁻⁸ M, 3×10⁻⁸ M, 2×10⁻⁸ M, 1×10⁻⁸ M, 9×10⁻⁹ M, 8×10⁻⁹ M, 7×10⁻⁹ M, 6×10⁻⁹ M, 5×10⁻⁹ M, 4×10⁻⁹ M, 3×10⁻⁹ M, 2×10⁻⁹ M, 1×10⁻⁹ M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M, 3×10⁻¹⁰ M, 2×10⁻¹⁰ M, 1×10⁻¹⁰ M or less.

In one embodiment, the peptides according to the present invention specifically bind to at least one binding site within CD38.

In one embodiment, the compound according to the present invention specifically binds to at least one binding site within human CD38.

In one embodiment, said binding site is linear. In one embodiment, said binding site is conformational.

In one embodiment, said binding site comprises 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 contiguous or non-contiguous amino acid residues.

In one embodiment, said binding site extends at least throughout amino acid residue 1 to amino acid residue 300 of human CD38 having SEQ ID NO: 3, or throughout the corresponding amino acid residues in homologous CD38 from other species.

In one embodiment, said binding site extends at least throughout amino acid residue 1 to amino acid residue 296 of human CD38 having SEQ ID NO: 3, or throughout the corresponding amino acid residues in homologous CD38 from other species.

In one embodiment, said binding site extends at least throughout amino acid residue 1 to amino acid residue 294 of human CD38 having SEQ ID NO: 3, or throughout the corresponding amino acid residues in homologous CD38 from other species.

In one embodiment, said binding site extends at least throughout amino acid residue 1 to amino acid residue 285 of human CD38 having SEQ ID NO: 3, or throughout the corresponding amino acid residues in homologous CD38 from other species.

In one embodiment, said binding site extends at least throughout amino acid residue 70 to amino acid residue 300 of human CD38 having SEQ ID NO: 3, or throughout the corresponding amino acid residues in homologous CD38 from other species.

In one embodiment, said binding site extends at least throughout amino acid residue 70 to amino acid residue 296 of human CD38 having SEQ ID NO: 3, or throughout the corresponding amino acid residues in homologous CD38 from other species.

In one embodiment, said binding site extends at least throughout amino acid residue 70 to amino acid residue 294 of human CD38 having SEQ ID NO: 3, or throughout the corresponding amino acid residues in homologous CD38 from other species.

In one embodiment, said binding site extends at least throughout amino acid residue 70 to amino acid residue 285 of human CD38 having SEQ ID NO: 3, or throughout the corresponding amino acid residues in homologous CD38 from other species.

In one embodiment, said binding site extends at least throughout amino acid residue 189 to amino acid residue 300 of human CD38 having SEQ ID NO: 3, or throughout the corresponding amino acid residues in homologous CD38 from other species.

In one embodiment, said binding site extends at least throughout amino acid residue 189 to amino acid residue 296 of human CD38 having SEQ ID NO: 3, or throughout the corresponding amino acid residues in homologous CD38 from other species.

In one embodiment, said binding site extends at least throughout amino acid residue 189 to amino acid residue 294 of human CD38 having SEQ ID NO: 3, or throughout the corresponding amino acid residues in homologous CD38 from other species.

In one embodiment, said binding site extends at least throughout amino acid residue 189 to amino acid residue 285 of human CD38 having SEQ ID NO: 3, or throughout the corresponding amino acid residues in homologous CD38 from other species.

In one embodiment, said binding site extends at least throughout amino acid residue 219 to amino acid residue 300 of human CD38 having SEQ ID NO: 3, or throughout the corresponding amino acid residues in homologous CD38 from other species.

In one embodiment, said binding site extends at least throughout amino acid residue 219 to amino acid residue 296 of human CD38 having SEQ ID NO: 3, or throughout the corresponding amino acid residues in homologous CD38 from other species.

In one embodiment, said binding site extends at least throughout amino acid residue 219 to amino acid residue 294 of human CD38 having SEQ ID NO: 3, or throughout the corresponding amino acid residues in homologous CD38 from other species.

In one embodiment, said binding site extends at least throughout amino acid residue 219 to amino acid residue 285 of human CD38 having SEQ ID NO: 3, or throughout the corresponding amino acid residues in homologous CD38 from other species.

In one embodiment, said binding site extends at least throughout amino acid residue 220 to amino acid residue 300 of human CD38 having SEQ ID NO: 3, or throughout the corresponding amino acid residues in homologous CD38 from other species.

In one embodiment, said binding site extends at least throughout amino acid residue 220 to amino acid residue 296 of human CD38 having SEQ ID NO: 3, or throughout the corresponding amino acid residues in homologous CD38 from other species.

In one embodiment, said binding site extends at least throughout amino acid residue 220 to amino acid residue 294 of human CD38 having SEQ ID NO: 3, or throughout the corresponding amino acid residues in homologous CD38 from other species.

In one embodiment, said binding site extends at least throughout amino acid residue 220 to amino acid residue 285 of human CD38 having SEQ ID NO: 3, or throughout the corresponding amino acid residues in homologous CD38 from other species.

In one embodiment, said binding site extends at least throughout amino acid residue 233 to amino acid residue 300 of human CD38 having SEQ ID NO: 3, or throughout the corresponding amino acid residues in homologous CD38 from other species.

In one embodiment, said binding site extends at least throughout amino acid residue 233 to amino acid residue 296 of human CD38 having SEQ ID NO: 3, or throughout the corresponding amino acid residues in homologous CD38 from other species.

In one embodiment, said binding site extends at least throughout amino acid residue 233 to amino acid residue 294 of human CD38 having SEQ ID NO: 3, or throughout the corresponding amino acid residues in homologous CD38 from other species.

In one embodiment, said binding site extends at least throughout amino acid residue 233 to amino acid residue 285 of human CD38 having SEQ ID NO: 3, or throughout the corresponding amino acid residues in homologous CD38 from other species.

In one embodiment, said binding site extends at least throughout amino acid residue 220 to amino acid residue 241 and amino acid residue 273 to amino acid residue 285 of human CD38 having SEQ ID NO: 3, or throughout the corresponding amino acid residues in homologous CD38 from other species.

In one embodiment, said binding site comprises at least cysteine 254 of human CD38 having SEQ ID NO: 3 or the corresponding cysteine in homologous CD38 from other species.

In one embodiment, said binding site comprises at least cysteine 275 of human CD38 having SEQ ID NO: 3 or the corresponding cysteine in homologous CD38 from other species.

In one embodiment, said binding site comprises at least cysteine 254 and cysteine 275 of human CD38 having SEQ ID NO: 3 or the corresponding cysteine in homologous CD38 from other species.

In one embodiment, said binding site comprises the 5^{th} (penultimate) C-terminal disulfide loop involving cysteine 254 and cysteine 275 of human CD38 having SEQ ID NO: 3 or the corresponding cysteine in homologous CD38 from other species. In particular, the 5^{th} (penultimate) C-terminal disulfide loop of human CD38 comprises amino acid residues 220 to amino acid residue 285 of human CD38 having SEQ ID NO: 3, or the corresponding cysteine in homologous CD38 from other species.

In one embodiment, said binding site does not comprise cysteine 287 of human CD38 having SEQ ID NO: 3 or the corresponding cysteine in homologous CD38 from other species.

In one embodiment, said binding site does not comprise cysteine 290 of human CD38 having SEQ ID NO: 3 or the corresponding cysteine in homologous CD38 from other species.

In one embodiment, said binding site does not comprise cysteine 287 and cysteine 290 of human CD38 having SEQ ID NO: 3 or the corresponding cysteine in homologous CD38 from other species.

In one embodiment, said binding site does not comprise the 6^{th} C-terminal disulfide loop involving cysteine 287 and cysteine 296 of human CD38 having SEQ ID NO: 3 or the corresponding cysteine in homologous CD38 from other species. In particular, the 6^{th} C-terminal disulfide loop of human CD38 comprises amino acid residues 285 to amino acid residue 300 of human CD38 having SEQ ID NO: 3, or the corresponding cysteine in homologous CD38 from other species.

In one embodiment, the peptides according to the present invention directly protect dopaminergic neurons from mitochondrial inhibition induced by the neurotoxin MPP⁺.

In one embodiment, the peptides according to the present invention have at least one, preferably at least two, more preferably the three following properties:
they protect dopaminergic neurons against energy deficit due to mitochondrial complex I inhibition, in particular against the mitochondrial neurotoxin MPP⁺;
they increase the release of the anti-inflammatory cytokine interleukin-10 (IL-10) in vitro using human peripheral blood mononuclear cells (PBMC); and/or
they increase IL-10 levels in vivo when injected to mice.

In one embodiment, the peptides according to the present invention compete with endogenous CD31 for CD38 binding, preferably with endogenous human CD31, and induce tyrosine phosphorylation. In particular, the peptides according to the present invention induce tyrosine phosphorylation of discrete cytoplasmic substrates that is inhibited in the presence of genistein.

Examples of discrete cytoplasmic substrates phosphorylated include, but are not limited to, the c-cbl proto-oncogene, the protein kinase syk, the p85 subunit of phosphatidylinositol-3 kinase, phospholipase C-γ (PLC-γ), the Raf-1/MAP kinase, the CD3-ζ/ZAP-70 signaling pathway.

In one embodiment, the peptides according to the present invention compete with endogenous CD31 for CD38 binding, preferably with endogenous human CD31, and trigger CD38 internalization.

In one embodiment, the peptides according to the present invention compete with endogenous CD31 for CD38 binding, preferably with endogenous human CD31, and induce lysosomal exocytosis. In particular, the peptides according to the present invention induce lysosomal exocytosis that is inhibited in the presence of vacuolin-1.

In one embodiment, the peptides according to the present invention compete with endogenous CD31 for CD38 binding, preferably with endogenous human CD31, and increase glucose uptake.

In one embodiment, the peptides according to the present invention comprise or consist of less than 195 amino acid residues, preferably less than 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30 or 25 amino acid residues.

In one embodiment, the peptides according to the present invention comprise or consist of less than 195 contiguous amino acid residues from human sCD31 having SEQ ID NO: 2 or an homolog thereof, preferably less than 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30 or 25 contiguous amino acid residues from human sCD31 having SEQ ID NO: 2 or an homolog thereof.

In one embodiment, the peptides according to the present invention comprise or consist of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 amino acid residues.

In one embodiment, the peptides according to the present invention comprise or consist of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 contiguous amino acid residues from human sCD31 having SEQ ID NO: 2 or an homolog thereof.

In one embodiment, the peptides according to the present invention comprise or consist of 5, 6, 7, 8, 9, 10, 11, 12, 13, 14, 15, 16, 17, 18, 19, 20, 21 or 22 amino acid residues.

It shall be understood that the length of the peptide as defined herein only takes into account the length of the CD31 peptide itself, excluding the length of a payload in the case of a CD31 peptide conjugate as described above.

In one embodiment, the peptides according to the present invention are not antibodies or antigen-binding fragment thereof.

The present invention also relates to a composition comprising, consisting of, or consisting essentially of, at least one peptide according to the present invention, which specifically binds to CD38.

The present invention also relates to a pharmaceutical composition comprising at least one peptide according to the present invention, which specifically binds to CD38; and at least one pharmaceutically acceptable excipient.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, e.g., FDA Office or EMA.

Pharmaceutically acceptable excipients that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., sodium carboxymethylcellulose), polyethylene glycol, polyacrylates, waxes, polyethylene-polyoxypropylene- block polymers, polyethylene glycol and wool fat.

In one embodiment, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The present invention also relates to a medicament comprising, consisting of, or consisting essentially of, at least one peptide according to the present invention, which specifically binds to CD38.

The present invention also relates to methods of preventing and/or treating a disease in a subject in need thereof, comprising administering to said subject a peptide according to the present invention, or the composition, the pharmaceutical composition or the medicament according to the present invention.

The present invention also relates to a peptide according to the present invention, or to the composition, the pharmaceutical composition or the medicament according to the present invention, for use as a drug.

The present invention also relates to a peptide according to the present invention, or to the composition, the pharmaceutical composition or the medicament according to the present invention, for use in preventing and/or treating a disease in a subject in need thereof.

In one embodiment, the disease is related to, due to, caused by, or characterized by, increased soluble CD38 levels in the subject. In particular, increased soluble CD38 levels may be detected in a blood sample (including whole blood, plasma and serum, preferably in a plasma sample), in a cerebrospinal fluid sample and/or in a biopsy sample.

By “increased soluble CD38 levels”, it is meant an amount or concentration of total soluble CD38 which is 10% higher, preferably 20%, 30%, 40%, 50% or more higher than in a substantially healthy subject, i.e., a subject known not to be affected with the disease at stake. Mean total soluble CD38 levels in substantially healthy human subjects are about 121 ± 7.8 U per mL of plasma (according to Lebedev et al., 2004. Burns. 30(6):552-6), or about 44.71 ± 4.3 pg per mL of plasma (according to Li et al., 2018. Anal Chim Acta. 1029:65-71) or up to 0.1 ng per mL of plasma (according to Zuch de Zafra et al., 2019. Clin Cancer Res. 25(13):3921-3933).

In one embodiment, the disease is related to, due to, caused by, or characterized by, reduced soluble CD31 levels in the subject. In particular, reduced soluble CD31 levels may be detected in a blood sample (including whole blood, plasma and serum, preferably in a plasma sample), in a cerebrospinal fluid sample and/or in a biopsy sample.

By “reduced soluble CD31 levels”, it is meant an amount or concentration of total soluble CD31 which is 10% lower, preferably 20%, 30%, 40%, 50% or more lower than in a substantially healthy subject, i.e., a subject known not to be affected with the disease at stake. Mean total soluble CD31 levels in substantially healthy human subjects are about 8.8 ± 4.8 ng per mL of plasma (according to Sato et al., 2001. J Rheumatol. 28(11):2460-5) or 12.87 ± 0.89 ng per mL of plasma (according to Fornasa et al., 2010. J Immunol. 184(10):5485-92) or 7.40 ± 3.27 ng per mL of plasma (according to Li et al., 2021. J Infect Dis. 223(1):178-179).

In one embodiment, the disease is related to, due to, caused by, or characterized by, reduced soluble CD31/soluble CD38 ratio in the subject. In particular, reduced soluble CD31/soluble CD38 ratio may be detected in a blood sample (including whole blood, plasma and serum, preferably in a plasma sample), in a cerebrospinal fluid sample and/or in a muscle biopsy.

In one embodiment, the disease is selected from the group comprising, or consisting of: inflammatory diseases; autoimmune diseases; metabolic and endocrine diseases; neurodegenerative diseases; neuroinflammatory diseases; cardiovascular diseases; kidney diseases; ocular diseases; age-related diseases and aging; fibrotic disorders; and cancer and metastasis.

In one embodiment, the disease is selected from the group comprising, or consisting of, a metabolic and endocrine disease, a neurodegenerative disease, a neuroinflammatory disease, an inflammatory disease, an autoimmune disease, and a combination thereof. In one embodiment, the disease is selected from the group comprising, or consisting of, a neurodegenerative disease, a neuroinflammatory disease, an inflammatory disease, an autoimmune disease, a metabolic and endocrine disease, and a combination thereof. In one embodiment, the disease is selected from the group comprising, or consisting of, an inflammatory disease, an autoimmune disease, a metabolic and endocrine disease, and a combination thereof. In one embodiment, the disease is selected from the group comprising, or consisting of, an autoimmune disease, a metabolic and endocrine disease, and a combination thereof.

It is to be understood that the present disclosure also encompasses symptoms of the recited diseases; hence preventing and/or treating a disease includes preventing the appearance or onset of at least one symptom associated with the disease and/or slowing down or stopping the progression, aggravation, or deterioration of at least one symptom of the disease and/or reducing or alleviating the severity of at least one symptom of the disease.

In one embodiment, the disease is an inflammatory disease.

Examples of inflammatory diseases include, but are not limited to, arthritis (including, e.g., osteoarthritis, rheumatoid arthritis, spondyloarthropathies and psoriatic arthritis); asthma (including, e.g., atopic asthma, nonatopic asthma, allergic asthma, exercise-induced asthma, drug-induced asthma, occupational asthma and late stage asthma); inflammatory bowel disease (including, e.g., Crohn’s disease, ulcerative colitis and colitis); inflammatory skin disorders (including, e.g., psoriasis, atopic dermatitis and contact hypersensitivity); multiple sclerosis; osteoporosis; tendonitis; allergic disorders (including, e.g., rhinitis, conjunctivitis and urticaria); inflammation in response to an insult to the host (including, e.g., injury or infection); transplant rejection; graft versus host disease; sepsis; and systematic lupus erythematosus.

In one embodiment, the disease is an autoimmune disease.

Examples of autoimmune diseases include, but are not limited to, alopecia areata; ankylosing spondylitis; arthritis; antiphospholipid syndrome; autoimmune Addison’s disease; autoimmune hemolytic anemia; autoimmune inner ear disease (also known as Ménière’s disease); autoimmune lymphoproliferative syndrome; autoimmune thrombocytopenic purpura; autoimmune hemolytic anemia; autoimmune hepatitis; Bechet’s disease; Crohn’s disease; diabetes mellitus type 1; glomerulonephritis; Graves’ disease; Guillain-Barré syndrome; inflammatory bowel disease; lupus nephritis; multiple sclerosis; myasthenia gravis; pemphigus; pernicous anemia; polyarteritis nodosa; polymyositis; primary biliary cirrhosis; psoriasis; Raynaud’s phenomenon; rheumatic fever; rheumatoid arthritis; scleroderma; Sjögren’s syndrome; systemic lupus erythematosus; ulcerative colitis; vitiligo; and Wegener’s granulomatosis.

In one embodiment, the disease is a metabolic and endocrine disease.

Examples of metabolic and endocrine diseases include, but are not limited to, diabetes (e.g., type 1 diabetes, type 2 diabetes, gestational diabetes) and diabetic complications; hyperglycemia; insulin resistance; impaired glucose tolerance; hyperinsulinism; diabetic complication; dyslipidemia; hypercholesterolemia; hypertriglyceridemia; HDL hypocholesterolemia; LDL hypercholesterolemia and/or HLD non-cholesterolemia; VLDL hyperproteinemia; dyslipoproteinemia; apolipoprotein A-1 hypoproteinemia; metabolic syndrome; syndrome X; obesity; non-alcoholic fatty liver disease (NAFLD); non-alcoholic steatohepatitis; and adrenal leukodystrophy.

In one embodiment, the metabolic and endocrine disease is a diabetic complication. By “diabetic complication”, it is meant a condition occurring in patients as a consequence of type 2 diabetes. Examples of diabetic complications include, but not limited to, heart failure, atherosclerosis, arrhythmias, hypertension, stroke, diabetic nephropathy, diabetic neuropathy, diabetic retinopathy, cataracts, glaucoma, non-proliferative retinopathy, proliferative retinopathy, macular edema, foot ulcers, sarcopenia, diabetic dermopathy, ketoacidosis, and gastroparesis.

In one embodiment, the disease is a neurodegenerative disease.

Examples of neurodegenerative diseases include, but are not limited to, Parkinson’s disease and related disorders (including, e.g., Parkinson’s disease, Parkinson-dementia, autosomal recessive PARK2 and PARK6-linked Parkinsonism, atypical parkinsonian syndromes, including, progressive supranuclear palsy, corticobasal degeneration syndrome, Lewy bodies dementia, multiple system atrophy, Guadeloupean Parkinsonism and Lytigo-bodig disease); motor neuron diseases (including, e.g., amyotrophic lateral sclerosis, frontotemporal dementia, progressive bulbar palsy, pseudobulbar palsy, primary lateral sclerosis, progressive muscular atrophy, spinal muscular atrophy and post-polio syndrome); neuro-inflammatory diseases; Alzheimer’s disease and related disorders (including, e.g., early stage of an Alzheimer’s disorder, mild stage of an Alzheimer’s disorder, moderate stage of an Alzheimer’s disorder, mild to moderate stage of an Alzheimer’s disorder, advanced stage of an Alzheimer’s disorder, mild cognitive impairment, vascular dementia, mixed dementia, Pick’s disease, argyrophilic grain disease, posterior cortical atrophy, and Wernicke-Korsakoff syndrome); prion diseases; lysosomal storage diseases; leukodystrophies; Huntington’s disease; multiple sclerosis; Down syndrome; spinal and bulbar muscular atrophy; HIV-associated neurocognitive disorder; Tourette syndrome; autosomal dominant spinocerebellar ataxia; Friedreich’s ataxia; dentatorubral pallidoluysian atrophy; myotonic dystrophy; schizophrenia; age associated memory impairment; autism and autism spectrum disorders; attention-deficit hyperactivity disorder; chronic pain; alcohol-induced dementia; progressive non-fluent aphasia; semantic dementia; spastic paraplegia; fibromyalgia; post-Lyme disease; neuropathies; withdrawal symptoms; Alpers’ disease; cerebro-oculo-facio-skeletal syndrome; Wilson’s disease; Cockayne syndrome; Leigh’s disease; neurodegeneration with brain iron accumulation; dyskinesia; dystonia (including, e.g., status dystonicus, spasmodic torticollis, Meige’s syndrome, and blepharospasm); athetosis; chorea; choreoathetosis; opsoclonus myoclonus syndrome; myoclonus; myoclonic epilepsy; akathisia; tremor (including, e.g., essential tremor, and intention tremor); restless legs syndrome; stiff-person syndrome; alpha-methylacyl-CoA racemase deficiency; Andermann syndrome; Arts syndrome; Marinesco-Sjögren syndrome; mitochondrial membrane protein-associated neurodegeneration; pantothenate kinase-associated neurodegeneration; polycystic lipomembranous osteodysplasia with sclerosing leukoencephalopathy; riboflavin transporter deficiency neuronopathy; and ataxia telangiectasia.

In one embodiment, the disease is a neuroinflammatory disease.

Examples of neuroinflammatory diseases include, but are not limited to, multiple sclerosis; encephalitis (including, e.g., viral encephalitis, herpesviral encephalitis, limbic encephalitis, and encephalitis lethargica); myelitis (including, e.g., poliomyelitis, acute disseminated encephalomyelitis, transverse myelitis, myalgic encephalomyelitis, and neuromyelitis optica); meningoencephalitis; optic neuritis; tropical spastic paraparesis; cavernous sinus thrombosis; brain abscess; epidural abscess; and the like. In some instances, primary conditions with secondary neuroinflammation (e.g., traumatic brain injury with secondary neuroinflammation) may also be considered a neuroinflammatory disease.

In one embodiment, the disease is cancer.

Examples of cancers include those listed in the 10^{th} revision of the International Statistical Classification of Diseases and Related Health Problems (ICD), under chapter II, blocks C00 to D48.

Further examples of cancers include, but are not limited to, recurrent, metastatic or multidrug resistant cancer.

Further examples of cancers include, but are not limited to, adenofibroma, adenoma, agnogenic myeloid metaplasia, AIDS-related malignancies, ameloblastoma, anal cancer, angiofollicular mediastinal lymph node hyperplasia, angiokeratoma, angiolymphoid hyperplasia with eosinophilia, angiomatosis, anhidrotic ectodermal dysplasia, anterofacial dysplasia, apocrine metaplasia, apudoma, asphyxiating thoracic dysplasia, astrocytoma (including, e.g., cerebellar astrocytoma and cerebral astrocytoma), atriodigital dysplasia, atypical melanocytic hyperplasia, atypical metaplasia, autoparenchymatous metaplasia, basal cell hyperplasia, benign giant lymph node hyperplasia, bile duct cancer (including, e.g., extrahepatic bile duct cancer), bladder cancer, bone cancer, brain tumor (including, e.g., brain stem glioma, cerebellar astrocytoma glioma, malignant glioma, supratentorial primitive neuroectodermal tumors, visual pathway and hypothalamic glioma, ependymoma, medulloblastoma, gestational trophoblastic tumor glioma, and paraganglioma), branchionia, female breast cancer, male breast cancer, bronchial adenomas/carcinoids, bronchopulmonary dysplasia, cancer growths of epithelial cells, pre-cancerous growths of epithelial cells, metastatic growths of epithelial cells, carcinoid heart disease, carcinoid tumor (including, e.g., gastrointestinal carcinoid tumor), carcinoma (including, e.g., carcinoma of unknown primary origin, adrenocortical carcinoma, islet cells carcinoma, adeno carcinoma, adeoncortical carcinoma, basal cell carcinoma, basosquamous carcinoma, bronchiolar carcinoma, Brown-Pearce carcinoma, cystadenocarcinoma, ductal carcinoma, hepatocarcinoma, Krebs carcinoma, papillary carcinoma, oat cell carcinoma, small cell lung carcinoma, non-small cell lung carcinoma, squamous cell carcinoma, transitional cell carcinoma, Walker carcinoma, Merkel cell carcinoma, and skin carcinoma), cementoma, cementum hyperplasia, cerebral dysplasia, cervical cancer, cervical dysplasia, cholangioma, cholesteatoma, chondroblastoma, chondroectodermal dysplasia, chordoma, choristoma, chrondroma, cleidocranial dysplasia, colon cancer, colorectal cancer, local metastasized colorectal cancer, congenital adrenal hyperplasia, congenital ectodermal dysplasia, congenital sebaceous hyperplasia, connective tissue metaplasia, craniocarpotarsal dysplasia, craniodiaphysial dysplasia, craniometaphysial dysplasia, craniopharyngioma, cylindroma, cystadenoma, cystic hyperplasia (including, e.g., cystic hyperplasia of the breast), cystosarconia phyllodes, dentin dysplasia, denture hyperplasia, diaphysial dysplasia, ductal hyperplasia, dysgenninoma, dysplasia epiphysialis hemimelia, dysplasia epiphysialis multiplex, dysplasia epiphysialis punctate, ectodermal dysplasia, Ehrlich tumor, enamel dysplasia, encephaloophthalmic dysplasia, endometrial cancer (including, e.g., ependymoma and endometrial hyperplasia), ependymoma, epithelial cancer, epithelial dysplasia, epithelial metaplasia, esophageal cancer, Ewing’s family of tumors (including, e.g., Ewing’s sarcoma), extrahepatic bile duct cancer, eye cancer (including, e.g., intraocular melanoma and retinoblastoma), faciodigitogenital dysplasia, familial fibrous dysplasia of jaws, familial white folded dysplasia, fibroma, fibromuscular dysplasia, fibromuscular hyperplasia, fibrous dysplasia of bone, florid osseous dysplasia, focal epithelial hyperplasia, gall bladder cancer, ganglioneuroma, gastric cancer (including, e.g., stomach cancer), gastrointestinal carcinoid tumor, gastrointestinal tract cancer, gastrointestinal tumors, Gaucher’s disease, germ cell tumors (including, e.g., extracranial germ cell tumors, extragonadal germ cell tumors, and ovarian germ cell tumors), giant cell tumor, gingival hyperplasia, glioblastoma, glomangioma, granulosa cell tumor, gynandroblastoma, hamartoma, head and neck cancer, hemangioendothelioma, hemangioma, hemangiopericytoma, hepatocellular cancer, hepatoma, hereditary renal-retinal dysplasia, hidrotic ectodermal dysplasia, histiocytonia, histiocytosis, hypergammaglobulinemia, hypohidrotic ectodermal dysplasia, hypopharyngeal cancer, inflammatory fibrous hyperplasia, inflammatory papillary hyperplasia, intestinal cancers, intestinal metaplasia, intestinal polyps, intraocular melanoma, intravascular papillary endothelial hyperplasia, kidney cancer, laryngeal cancer, leiomyoma, leukemia (including, e.g., acute lymphoblastic leukemia, acute lymphocytic leukemia, acute myeloid leukemia, acute myelogenous leukemia, acute hairy cell leukemia, acute B-cell leukemia, acute T-cell leukemia, acute HTLV leukemia, chronic lymphoblastic leukemia, chronic lymphocytic leukemia, chronic myeloid leukemia, chronic myelogenous leukemia, chronic hairy cell leukemia, chronic B-cell leukemia, chronic T-cell leukemia, and chronic HTLV leukemia), Leydig cell tumor, lip and oral cavity cancer, lipoma, liver cancer, lung cancer (including, e.g., small cell lung cancer and non-small cell lung cancer), lymphangiomyoma, lymphaugioma, lymphoma (including, e.g., AIDS-related lymphoma, central nervous system lymphoma, primary central nervous system lymphoma, Hodgkin’s lymphoma, non-Hodgkin’s lymphoma, Hodgkin’s lymphoma during pregnancy, non-Hodgkin’s lymphoma during pregnancy, mast cell lymphoma, B-cell lymphoma, adenolymphoma, Burkitt’s lymphoma, cutaneous T-cell lymphoma, large cell lymphoma, and small cell lymphoma), lymphopenic thymic dysplasia, lymphoproliferative disorders, macroglobulinemia (including, e.g., Waldenstrom’s macroglobulinemia), malignant carcinoid syndrome, malignant mesothelioma, malignant thymoma, mammary dysplasia, mandibulofacial dysplasia, medulloblastoma, meningioma, mesenchymoma, mesonephroma, mesothelioma (including, e.g., malignant mesothelioma), metaphysial dysplasia, metaplastic anemia, metaplastic ossification, metaplastic polyps, metastatic squamous neck cancer (including, e.g., metastatic squamous neck cancer with occult primary), Mondini dysplasia, monostotic fibrous dysplasia, mucoepithelial dysplasia, multiple endocrine neoplasia syndrome, multiple epiphysial dysplasia, multiple myeloma/plasma cell neoplasm, mycosis fungoides, myelodysplastic syndrome, myeloid metaplasia, myeloproliferative disorders, chronic myeloproliferative disorders, myoblastoma, myoma, myxoma, nasal cavity and paranasal sinus cancer, nasopharyngeal cancer, prostatic neoplasm, colon neoplasm, abdomen neoplasm, bone neoplasm, breast neoplasm, digestive system neoplasm, liver neoplasm, pancreas neoplasm, peritoneum neoplasm, endocrine glands neoplasm (including, e.g., adrenal neoplasm, parathyroid neoplasm, pituitary neoplasm, testicles neoplasm, ovary neoplasm, thymus neoplasm, and thyroid neoplasm), eye neoplasm, head and neck neoplasm, nervous system neoplasm (including, e.g., central nervous system neoplasm and peripheral nervous system neoplasm), lymphatic system neoplasm, pelvic neoplasm, skin neoplasm, soft tissue neoplasm, spleen neoplasm, thoracic neoplasm, urogenital tract neoplasm, neurilemmoma, neuroblastoma, neuroepithelioma, neurofibroma, neurofibromatosis, neuroma, nodular hyperplasia of prostate, nodular regenerative hyperplasia, oculoauriculovertebral dysplasia, oculodentodigital dysplasia, oculovertebral dysplasia, odontogenic dysplasia, odontoma, opthalmomandibulomelic dysplasia, oropharyngeal cancer, osteoma, ovarian cancer (including, e.g., ovarian epithelial cancer and ovarian low malignant potential tumor), pancreatic cancer (including, e.g., islet cell pancreatic cancer and exocrine pancreatic cancer), papilloma, paraganglioma, nonchromaffin paraganglioma, paranasal sinus and nasal cavity cancer, paraproteinemias, parathyroid cancer, periapical cemental dysplasia, pheochromocytoma (including, e.g., penile cancer), pineal and supratentorial primitive neuroectodermal tumors, pinealoma, pituitary tumor, plasma cell neoplasm/multiple myeloma, plasmacytoma, pleuropulmonary blastoma, polyostotic fibrous dysplasia, polyps, pregnancy cancer, pre-neoplastic disorders (including, e.g., benign dysproliferative disorders such as benign tumors, fibrocystic conditions, tissue hypertrophy, intestinal polyps, colon polyps, esophageal dysplasia, leukoplakia, keratoses, Bowen’s disease, Farmer’s skin, solar cheilitis, and solar keratosis), primary hepatocellular cancer, primary liver cancer, primary myeloid metaplasia, prostate cancer, pseudoachondroplastic spondyloepiphysial dysplasia, pseudoepitheliomatous hyperplasia, purpura, rectal cancer, renal cancer (including, e.g., kidney cancer, renal pelvis, ureter cancer, transitional cell cancer of the renal pelvis and ureter), reticuloendotheliosis, retinal dysplasia, retinoblastoma, salivary gland cancer, sarcomas (including, e.g., uterine sarcoma, soft tissue sarcoma, carcinosarcoma, chondrosarcoma, fibrosarcoma, hemangiosarcoma, Kaposi’s sarcoma, leiomyosarcoma, liposarcoma, lymphangiosarcoma, myosarcoma, myxosarcoma, rhabdosarcoma, sarcoidosis sarcoma, osteosarcoma, Ewing sarcoma, malignant fibrous histiocytoma of bone, and clear cell sarcoma of tendon sheaths), sclerosing angioma, secondary myeloid metaplasia, senile sebaceous hyperplasia, septooptic dysplasia, Sertoli cell tumor, Sezary syndrome, skin cancer (including, e.g., melanoma skin cancer and nonmelanoma skin cancer), small intestine cancer, spondyloepiphysial dysplasia, squamous metaplasia (including, e.g., squamous metaplasia of amnion), stomach cancer, supratentorial primitive neuroectodermal and pineal tumors, supratentorial primitive neuroectodermal tumors, symptomatic myeloid metaplasia, teratoma, testicular cancer, theca cell tumor, thymoma (including, e.g., malignant thymoma), thyroid cancer, trophoblastic tumors (including, e.g., gestational trophoblastic tumors), ureter cancer, urethral cancer, uterine cancer, vaginal cancer, ventriculoradial dysplasia, verrucous hyperplasia, vulvar cancer, Waldenstrom’s macroglobulinemia, and Wilms’ tumor.

In one embodiment, the disease is a cardiovascular disease.

Examples of cardiovascular diseases include, but are not limited to, aneurysm, angina, arrhythmia, atherosclerosis, cardiomyopathy, diabetic cardiomyopathy, cerebrovascular disease, congenital heart disease, congestive heart failure, myocarditis, valve disease coronary, artery disease dilated, cardiomyopathy, diastolic dysfunction, pericarditis, endocarditis, heart attack, hypertension, hypertrophic cardiomyopathy, mitral valve prolapse, mitral valve stenosis, aortic stenosis, myocardial infarction, stroke, ischemia-reperfusion injury, venous thromboembolism, coarctation of the aorta, Kawasaki disease, giant cell arteritis, Takayasu arteritis, polyarteritis nodosa, ANCA-associated vasculitis, microscopic polyangiitis, Wegener’s disease, Churg-Strauss syndrome, anti-glomerular basement membrane (GBM) disease, Henoch-Schönlein purpura, cryoglobulinemic vasculitis, hypocomplementemic urticarial vasculitis, thromboangiitis obliterans, Behçet’s disease, Cogan’s disease, cutaneous leukocytoclastic angiitis, cutaneous arteritis, primary central nervous system vasculitis, isolated aortitis, lupus vasculitis, rheumatoid vasculitis, sarcoid vasculitis, hepatitis C virus-associated cryoglobulinemic vasculitis, hepatitis B virus-associated vasculitis, syphilis-associated aortitis, drug-associated immune complex vasculitis, drug-associated ANCA-associated vasculitis, and cancer-associated vasculitis.

In one embodiment, the disease is a kidney disease.

Examples of kidney diseases include, but are not limited to, nephritis, nephrosis, acute renal failure, chronic renal failure, diabetic nephropathy, acute kidney injury, polycystic kidney disease, chronic kidney disease, end-stage renal disease, acute tubular necrosis, renal cortical necrosis, renal artery stenosis, renal ischemia, hypertensive nephropathy, renovascular hypertension, renal tubular acidosis, pyelonephritis, Balkan endemic nephropathy, minimal change disease, focal segmental glomerulosclerosis, membranous glomerulonephritis, mesangial proliferative glomerulonephritis, endocapillary proliferative glomerulonephritis, and membranoproliferative glomerulonephritis.

In one embodiment, the disease is an ocular disease.

Examples of ocular diseases include, but are not limited to, age-related macular degeneration, glaucoma, macular edema, diabetic retinopathy, cataract, non-proliferative retinopathy, proliferative retinopathy, macular edema, dry eye syndrome, uveitis, allergic conjunctivitis, scleritis, keratitis, keratoconjunctivitis, vernal keratoconjunctivitis, atopic keratoconjunctivitis, cicatrizing conjunctivitis, blepharitis, endophthalmitis, retinitis, retinopathies, choroiditis, Sjogren’s syndrome and retinal necrosis.

In one embodiment, the disease is an age-related disease.

Examples of age-related diseases include, but are not limited to, benign tumors; cardiovascular diseases, such as stroke, atherosclerosis and hypertension; angioma; osteoporosis; insulin-resistance and type 2 diabetes, including diabetic retinopathy and neuropathy; Alzheimer’s disease; Parkinson’s disease; age-related macular degeneration; arthritis; seborrheic keratosis; actinic keratosis; photoaged skin; skin spots; skin cancer; systemic lupus erythematosus; psoriasis; smooth muscle cell proliferation and intimal thickening following vascular injury; inflammation; arthritis; side effects of chemotherapy; benign prostatic hyperplasia; as well as less common diseases wherein their incidence is higher in elderly people than in young people.

In one embodiment, the disease is aging.

“Aging”, also referred to as “organismal aging”, is manifested by age-related diseases, the incidence of which increases with age. Death from aging means death from age-related diseases. Suppression or alleviation of aging delays one, some or most age-related diseases. Slow aging is manifested by delayed age-related diseases. Slow aging is considered to be a type of healthy aging.

In one embodiment, the methods according to the present invention are for delaying organismal aging.

In one embodiment, the peptide according to the present invention, or the composition, the pharmaceutical composition or the medicament according to the present invention, is for use in delaying organismal aging.

In one embodiment, the disease is a fibrotic disorder.

By “fibrotic disorder”, it is meant a disease characterized by the development of fibrosis in a single or several organs, including, without limitation, lungs, liver, brain, heart, kidney, skin, and joints.

As used herein, the term “fibrosis” refers to a pathological wound healing in which an excessive accumulation of extracellular matrix components, such as the collagen, leads to connective tissue replacing normal parenchymal tissue.

Examples of fibrotic disorders affecting lungs include, but are not limited to, cystic fibrosis, chronic obstructive pulmonary disease (COPD), acute respiratory distress syndrome (ARDS), combined pulmonary fibrosis and emphysema (CPFR), pulmonary edema, pneumonia, coronavirus pneumonia, eosinophilic pneumonia, respiratory hypersensitivity, allergic bronchopulmonary aspergillosis (ABPA), hypersensitivity pneumonitis (HP), interstitial lung disease (ILD), diffuse parenchymal lung disease, pneumoconiosis, extrinsic allergic alveolitis, iatrogenic ILD caused by drugs and/or radiation, post-infectious ILD, sarcoidosis, idiopathic interstitial pneumonia, idiopathic pulmonary fibrosis (IPF), pulmonary fibrosis with a histopathological pattern of usual interstitial pneumonia, nonspecific interstitial pneumonia, respiratory bronchiolitis ILD, desquamative interstitial pneumonia, cryptogenic organizing pneumonia, lymphoid interstitial pneumonia, and acute interstitial pneumonia.

Examples of fibrotic disorders affecting the liver include, but are not limited to, hepatitis, chronic cholestatic disease, progressive familial intrahepatic cholestasis (PFIC), primary biliary cholangitis (PBC), primary sclerosing cholangitis (PSC), alcoholic and non-alcoholic fatty liver disease, non-alcoholic steatohepatitis (NASH), autoimmune hepatitis, and cirrhosis.

Examples of fibrotic disorders affecting the heart include, but are not limited to, Löffler’s endomyocarditis, endomyocardial fibrosis, heart failure, valvular disease, arrhythmia, diabetic hypertrophic cardiomyopathy, idiopathic dilated cardiomyopathy, coronary heart disease, hypertensive left ventricular hypertrophy, healed myocarditis, dilated cardiomyopathies (DCMs), and myotonic dystrophy.

Examples of fibrotic disorders affecting skin include, but are not limited to, keloid disorders, systemic sclerosis, scleroderma, nephrogenic fibrosing dermopathy, mixed connective tissue disease, scleromyxedema, scleredema, sclerodermatous graft vs. host disease, eosinophilia-myalgia syndrome, and eosinophilic fasciitis

Examples of fibrotic disorders affecting the kidney include, but are not limited to, diabetic nephropathy, hypertensive nephrosclerosis, allograft nephropathy, vasculitis, lupus nephritis, immune complex glomerulonephritis, and post-infectious glomerulonephritis, focal segmental glomerulosclerosis, hypertensive glomerulosclerosis, collapsing glomerulopathy, primary membranous nephropathy, thin basement membrane disease, interstitial nephritis, minimal change disease, pyelonephritis, polycystic kidney disease, urinary tract infections, congenital nephrotic syndrome, congestive renal failure, cystinuria, glomerulopathy, glomerulonephritis, and Sézary syndrome.

Examples of fibrotic disorders affecting other organs include, but are not limited to, rheumatoid arthritis, myelopathy, sarcopaenia, eosinophilic esophagitis, hypereosinophilic syndromes (HES), Hamman-Rich syndrome, myelofibrosis, mediastinal fibrosis, obesity, type 2 diabetes mellitus chronic heart failure with liver congestion, hereditary hemochromatosis, amyloidosis, and chemotherapy- or radiation-induced fibrosis.

In one embodiment, the disease is selected from the group comprising or consisting of type 2 diabetes; diabetic complications; rheumatoid arthritis; systemic lupus erythematosus; obesity; ulcerative colitis; non-alcoholic steatohepatitis; chronic kidney disease; age-related macular degeneration; and heart failure.

In one embodiment, the disease is selected from the group comprising or consisting of type 2 diabetes; rheumatoid arthritis; systemic lupus erythematosus; Crohn’s disease; ulcerative colitis; atherosclerosis; heart failure; diabetic cardiopathy; diabetic nephropathy; chronic kidney disease; sarcopenia; systemic sclerosis; idiopathic pulmonary fibrosis; obesity; non-alcoholic steatohepatitis; diabetic retinopathy; age-related macular degeneration; glaucoma; dry eye syndrome; and chemotherapy- or radiation-induced fibrosis.

In one embodiment, the disease is selected from the group comprising or consisting of multiple sclerosis; rheumatoid arthritis; systemic lupus erythematosus; diabetes; obesity; non-alcoholic steatohepatitis; amyotrophic lateral sclerosis; Parkinson’s disease and related disorders; Alzheimer’s disease and related disorders; Huntington disease; Gaucher’s disease; age-related macular degeneration; and glaucoma.

In one embodiment, the disease is selected from the group comprising or consisting of multiple sclerosis; rheumatoid arthritis; systemic lupus erythematosus; diabetes; obesity; and non-alcoholic steatohepatitis.

In one embodiment, the disease is selected from the group comprising or consisting of amyotrophic lateral sclerosis; Parkinson’s disease and related disorders; Alzheimer’s disease and related disorders; and multiple sclerosis.

In some embodiments, the disease is multiple sclerosis. In some embodiments, the disease is rheumatoid arthritis. In some embodiments, the disease is systemic lupus erythematosus. In some embodiments, the disease is diabetes. In some embodiments, the disease is obesity. In some embodiments, the disease is non-alcoholic steatohepatitis. In some embodiments, the disease is amyotrophic lateral sclerosis. In some embodiments, the disease is Parkinson’s disease and related disorders. In some embodiments, the disease is Alzheimer’s disease and related disorders. In some embodiments, the disease is Huntington disease. In some embodiments, the disease is Gaucher’s disease. In some embodiments, the disease is age-related macular degeneration. In some embodiments, the disease is glaucoma. In some embodiments, the disease is atherosclerosis. In some embodiments, the disease is chemotherapy- or radiation-induced fibrosis. In some embodiments, the disease is chronic kidney disease. In some embodiments, the disease is Crohn’s disease. In some embodiments, the disease is diabetic cardiomyopathy. In some embodiments, the disease is diabetic complications. In some embodiments, the disease is diabetic nephropathy. In some embodiments, the disease is diabetic retinopathy. In some embodiments, the disease is dry eye syndrome. In some embodiments, the disease is heart failure. In some embodiments, the disease is idiopathic pulmonary fibrosis. In some embodiments, the disease is rheumatoid arthritis. In some embodiments, the disease is sarcopenia. In some embodiments, the disease is systemic sclerosis. In some embodiments, the disease is type 2 diabetes. In some embodiments, the disease is ulcerative colitis.

The present invention also relates to methods of reducing the severity of fibrosis and/or slowing down the progression of fibrosis and/or preventing the development of fibrosis in a subject in need thereof, comprising administering to said subject a peptide according to the present invention, or the composition, the pharmaceutical composition or the medicament according to the present invention.

The present invention also relates to a peptide according to the present invention, or to the composition, the pharmaceutical composition or the medicament according to the present invention, for use in reducing the severity of fibrosis and/or slowing down the progression of fibrosis and/or preventing the development of fibrosis in a subject in need thereof.

The severity or the progression of fibrosis can be readily measured by the attending physician using means and methods well known in the art, include, without limitation, hematology analyses, physiological examinations, serum markers of fibrosis, magnetic resonance elastography (MRE), and ultrasound/transient elastography.

The present invention also relates to methods of increasing the level of at least one anti-inflammatory cytokine in a subject in need thereof, in particular in the blood of said subject, comprising administering to said subject a peptide according to the present invention, or the composition, the pharmaceutical composition or the medicament according to the present invention.

The present invention also relates to a peptide according to the present invention, or to the composition, the pharmaceutical composition or the medicament according to the present invention, for use in increasing the level of at least one anti-inflammatory cytokine in a subject in need thereof, in particular in the blood of said subject.

Examples of anti-inflammatory cytokines include, but are not limited to, interleukin-10 (IL-10), transforming growth factor β (TGF-β), interleukin-1ra (IL-1ra), interleukin-4 (IL-4), interleukin-6 (IL-6), interleukin-11 (IL-11), interleukin-13 (IL-13) and interleukin-22 (IL-22).

Methods to measure blood cytokine levels in a subject are known in the art. Such methods include, for example, the use of ELISA test, such as the one described in the Example section below.

In particular, the present invention relates to a method of increasing the level of IL-10 in a subject in need thereof, in particular in the blood of said subject, comprising administering to said subject a peptide according to the present invention, or the composition, the pharmaceutical composition or the medicament according to the present invention.

In particular, the present invention relates to a peptide according to the present invention, or to the composition, the pharmaceutical composition or the medicament according to the present invention, for use in increasing the level of IL-10 in a subject in need thereof, in particular in the blood of said subject.

Methods to measure IL-10 levels in a blood sample from a subject are well known in the art and include, e.g., the use of an ELISA test.

In one embodiment, the peptide according to the present invention, or the composition, the pharmaceutical composition or the medicament according to the present invention triggers an increase of IL-10 levels in the blood of a subject by at least 100%, 200%, 300%, in particular at least 400% and more particularly at least 500%, as compared to the administration of negative control molecule.

The present invention also relates to methods of lowering glucose levels in a subject in need thereof, in particular in the blood of said subject, comprising administering to said subject a peptide according to the present invention, or the composition, the pharmaceutical composition or the medicament according to the present invention.

The present invention also relates to a peptide according to the present invention, or to the composition, the pharmaceutical composition or the medicament according to the present invention, for use in lowering glucose levels in a subject in need thereof, in particular in the blood of said subject.

Methods to measure blood glucose levels in a subject are known in the art, and include, without limitation, chemical methods (such as, e.g., the Folin-Wu method, the Benedict’s method, the Nelson-Somogyi method, the neocuproine method, the Shaeffer-Hartmann-Somogyi method, the Hagedorn-Jensen method, the ortho-toluidine method, and the anthrone method) and enzymatic methods (such as, e.g., the Saifer-Gerstenfeld method, the Trinder method, the Kodak Ektachem method, and the use of glucometers, either electronic or in the form of strips impregnated with a glucose oxidase reagent).

In humans, so-called “normal” blood glucose levels range from about 4.4 mmol/L to about 6.1 mmol/L, that is to say from about 79 mg/dL to about 110 mg/dL.

In one embodiment, the expression “lowering glucose levels” refer to reducing or decreasing the level or measurement (when compared to baseline) of one or more of (i) fasting whole blood glucose test; (ii) fasting plasma glucose test; (iii) fasting serum glucose test; (iv) non-fasting whole blood glucose test; (v) non-fasting plasma glucose test; (vi) non-fasting serum glucose test; or (vii) OGTT, by any amount compared to baseline. In one embodiment, a decrease in blood glucose level is determined relative to the starting blood glucose level of the subject (for example, prior to treatment with a peptide according to the present invention, or the composition, the pharmaceutical composition or the medicament according to the present invention).

In one embodiment, the glucose level is lowered or reduced by at least 1 mg/dL, such as by 5 mg/dL, 10 mg/dL, 20 mg/dL, 30 mg/dL, 40 mg/dL, 50 mg/dL, 60 mg/dL, 70 mg/dL, 80 mg/dL, 90 mg/dL, 100 mg/dL or more.

Depending on the subject in need thereof, the uses and methods described above can be for human or veterinary applications.

In one embodiment, the subject is an animal.

Examples of animals include, but are not limited to, mammals, birds, reptiles, amphibians, fishes, insects and mollusks.

In one embodiment, the subject is a non-human animal, including, but not limited to, a farm animal - or an animal of agricultural value (such as, e.g., cattle, cows, bison, pigs, swine, sheep, goats, horses, donkeys, alpacas, llamas, deer, elks, moose, ostriches, emus, ducks, geese, chickens, partridges, quails, pheasants, minks, salmons, codfishes, catfishes, herrings, trout, basses, perches, flounders, sharks, tuna fishes, cancers, lobsters, crayfishes, snails, clams, oysters, and the like), a companion animal (such as, e.g., dog, cats, rabbits, rodents, fishes, snakes and the like), and a non-human primate (such as, e.g., great apes including chimpanzees, gorillas, and orangutans; lesser apes, including gibbons; Old World monkeys; New World monkeys; and prosimians, including tarsiers, lemurs, and lorises).

In one embodiment, the non-human animal is selected from the group comprising or consisting of dogs, cats, and cows.

In one embodiment, the subject is a human.

In one embodiment, the subject is an adult (e.g., a subject above the age of 18 in human years or a subject after reproductive capacity has been attained). In another embodiment, the subject is a child (for example, a subject below the age of 18 in human years or a subject before reproductive capacity has been attained).

In one embodiment, the subject is a male. In one embodiment, the subject is a female.

In one embodiment, the subject is/was diagnosed with a disease selected from the group comprising or consisting of inflammatory diseases; autoimmune diseases; metabolic and endocrine diseases; neurodegenerative diseases; neuroinflammatory diseases; cardiovascular diseases; kidney diseases; ocular diseases; age-related diseases; fibrotic disorders; and cancer and metastasis.

In one embodiment, the subject is/was diagnosed with a disease selected from the group comprising or consisting of age-related macular degeneration; Alzheimer’s disease and related disorders; amyotrophic lateral sclerosis; atherosclerosis; chemotherapy- or radiation-induced fibrosis; chronic kidney disease; Crohn’s disease; diabetes; diabetic cardiomyopathy; diabetic complications; diabetic nephropathy; diabetic retinopathy; dry eye syndrome; Gaucher’s disease; glaucoma; heart failure; Huntington disease; idiopathic pulmonary fibrosis; multiple sclerosis; non-alcoholic steatohepatitis; obesity; Parkinson’s disease and related disorders; rheumatoid arthritis rheumatoid arthritis; sarcopenia; systemic lupus erythematosus; systemic sclerosis; and type 2 diabetes. ulcerative colitis.

In one embodiment, the subject is at risk of developing a disease selected from the group comprising or consisting of inflammatory diseases; autoimmune diseases; metabolic and endocrine diseases; neurodegenerative diseases; neuroinflammatory diseases; cardiovascular diseases; kidney diseases; ocular diseases; age-related diseases; fibrotic disorders; and cancer and metastasis.

In one embodiment, the subject is at risk of developing a disease selected from the group comprising or consisting of age-related macular degeneration; Alzheimer’s disease and related disorders; amyotrophic lateral sclerosis; atherosclerosis; chemotherapy- or radiation-induced fibrosis; chronic kidney disease; Crohn’s disease; diabetes; diabetic cardiomyopathy; diabetic complications; diabetic nephropathy; diabetic retinopathy; dry eye syndrome; Gaucher’s disease; glaucoma; heart failure; Huntington disease; idiopathic pulmonary fibrosis; multiple sclerosis; non-alcoholic steatohepatitis; obesity; Parkinson’s disease and related disorders; rheumatoid arthritis rheumatoid arthritis; sarcopenia; systemic lupus erythematosus; systemic sclerosis; and type 2 diabetes..

In one embodiment, the subject is/was diagnosed with increased plasma soluble CD38 levels.

In one embodiment, the subject is/was diagnosed with increased cerebrospinal fluid soluble CD38 levels.

In one embodiment, the subject is/was diagnosed with increased soluble CD38 levels in a biopsy sample.

Methods to detect and measure soluble CD38 levels are well known to the skilled artisan. In particular, means and kits for detecting and measuring soluble CD38 levels are commercially available.

In one embodiment, the subject is/was diagnosed with reduced plasma soluble CD31 levels.

In one embodiment, the subject is/was diagnosed with reduced cerebrospinal fluid soluble CD31 levels.

In one embodiment, the subject is/was diagnosed with reduced soluble CD31 levels in a biopsy sample.

Methods to detect and measure soluble CD31 levels are well known to the skilled artisan. In particular, means and kits for detecting and measuring soluble CD31 levels are commercially available.

In one embodiment, the subject is/was diagnosed with reduced plasma soluble CD31/soluble CD38 ratio.

In one embodiment, the subject is/was diagnosed with reduced cerebrospinal fluid soluble CD31/soluble CD38 ratio.

In one embodiment, the subject is/was diagnosed with reduced soluble CD31/soluble CD38 ratio in a biopsy sample.

Methods to detect and measure soluble CD31 and soluble CD38 levels are well known to the skilled artisan. In particular, means and kits for detecting and measuring soluble CD31 and soluble CD38 levels are commercially available.

In one embodiment, the peptide, composition, pharmaceutical composition or medicament according to the present invention will be formulated for administration to the subject.

In one embodiment, the peptide, composition, pharmaceutical composition or medicament according to the present invention is to be administered systemically or locally.

In one embodiment, the peptide, composition, pharmaceutical composition or medicament according to the present invention is to be administered by injection, orally, topically, nasally, buccally, rectally, vaginaly, intratracheally, by endoscopy, transmucosally, or by percutaneous administration.

In one embodiment, the peptide, composition, pharmaceutical composition or medicament according to the present invention is to be injected, preferably systemically injected.

Examples of formulations adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Examples of systemic injections include, but are not limited to, intravenous (iv), subcutaneous (sc), intradermal (id), intramuscular (im), intraarterial, intraparenteral, intranodal, intralymphatic, intraperitoneal (ip), intracranial, intracardiac, intralesional, intraprostatic, intravaginal, intrarectal, intrathecal, intranasal, intratumoral (it), intravesicular, and perfusion.

In one embodiment, when injected, the peptide, composition, pharmaceutical composition or medicament according to the present invention is sterile. Methods for obtaining a sterile composition include, but are not limited to, GMP synthesis (where GMP stands for “Good manufacturing practice”).

Sterile injectable forms of a composition may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer’s solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In one embodiment, the peptide, composition, pharmaceutical composition or medicament according to the present invention is to be administered orally. Example of formulations adapted for oral administration include, but are not limited to, solid forms, liquid forms and gels. Examples of solid forms adapted to oral administration include, but are not limited to, tablets, capsules (each of which includes sustained-release or timed-release formulations), pills, powders, granules, wafers, as well as effervescent tablet and any other solid forms suitable for solution in, or suspension in, liquid prior to oral administration. Examples of liquid forms adapted to oral administration include, but are not limited to, solutions, suspensions, emulsions, elixirs, tinctures, potions, syrups and liquors.

It will be understood that other suitable routes of administration are also contemplated in the present invention, and the administration mode will ultimately be decided by the attending physician within the scope of sound medical judgment. Apart from administration by injection (iv, ip, im and the like), other routes are available, such as nebulization (Respaud et al., 2014. MAbs. 6(5):1347-55; Guilleminault et al., 2014. J Control Release. 196:344-54; Respaud et al., 2015. Expert Opin Drug Deliv. 12(6):1027-39) or subcutaneous administration (Jackisch et al., 2014. Geburtshilfe Frauenheilkd. 74(4):343-349; Solal-Celigny, 2015. Expert Rev Hematol. 8(2):147-53).

In one embodiment, the peptide, composition, pharmaceutical composition or medicament according to the present invention is able to cross the blood-brain barrier (BBB), preferably when using an oral, intravenous or subcutaneous route of administration.

In one embodiment, the peptide, composition, pharmaceutical composition or medicament according to the present invention is to be administered to the subject in need thereof in a therapeutically effective amount.

Such therapeutically amount can be determined by the skilled artisan by routine tests including assessment of the effect of administration of the peptide, composition, pharmaceutical composition or medicament on the diseases which are sought to be prevented and/or to be treated by the administration of said peptide, composition, pharmaceutical composition or medicament according to the present invention.

For example, such tests can be implemented by analyzing both quantitative and qualitative effect of the administration of different amounts of said aforementioned peptide, composition, pharmaceutical composition or medicament according to the present invention on a set of markers (biological and/or clinical) characteristics of said diseases, in particular from a biological sample of a subject.

It will be however understood that the total daily usage of the peptide, composition, pharmaceutical composition or medicament according to the present invention will be decided by the attending physician within the scope of sound medical judgment. The specific therapeutically effective dose level for any particular patient will depend upon a variety of factors including the disease being treated and the severity of the disease; activity of the peptide, composition, pharmaceutical composition or medicament employed; the age, body weight, general health, sex and diet of the subject; the time of administration, route of administration, and rate of excretion of the peptide, composition, pharmaceutical composition or medicament employed; the duration of the treatment; drugs used in combination or coincidental with the peptide, composition, pharmaceutical composition or medicament employed; and like factors well known in the medical arts. For example, it is well within the skill of the art to start doses of the peptide at levels lower than those required to achieve the desired therapeutic effect and to gradually increase the dosage until the desired effect is achieved. The total dose required for each treatment may be administered by multiple doses or in a single dose.

In one embodiment, a therapeutically effective amount of the peptide, composition, pharmaceutical composition or medicament according to the present invention ranges from about 1 ng/kg to about 1 mg/kg, preferably from about 1 µg/kg to about 1 mg/kg. In one embodiment, a therapeutically effective amount of the peptide, composition, pharmaceutical composition or medicament according to the present invention is about 1 ng/kg, 5 ng/kg, 10 ng/kg, 25 ng/kg, 50 ng/kg, 75 ng/kg, 100 ng/kg, 150 ng/kg, 200 ng/kg, 250 ng/kg, 300 ng/kg, 350 ng/kg, 400 ng/kg, 450 ng/kg, 500 ng/kg, 550 ng/kg, 600 ng/kg, 650 ng/kg, 700 ng/kg, 750 ng/kg, 800 ng/kg, 850 ng/kg, 900 ng/kg, 950 ng/kg, 1 µg/kg, 5 µg/kg, 10 µg/kg, 25 µg/kg, 50 µg/kg, 75 µg/kg, 100 µg/kg, 150 µg/kg, 200 µg/kg, 250 µg/kg, 300 µg/kg, 350 µg/kg, 400 µg/kg, 450 µg/kg, 500 µg/kg, 550 µg/kg, 600 µg/kg, 650 µg/kg, 700 µg/kg, 750 µg/kg, 800 µg/kg, 850 µg/kg, 900 µg/kg, 950 µg/kg, 1 mg/kg.

In one embodiment, a therapeutically effective amount of the peptide, composition, pharmaceutical composition or medicament according to the present invention ranges from about 50 ng/day to about 100 mg/day, preferably from about 50 µg/day to about 100 mg/day. In one embodiment, a therapeutically effective amount of the peptide, composition, pharmaceutical composition or medicament according to the present invention is about 50 ng/day, 100 ng/day, 200 ng/day, 300 ng/day, 400 ng/day, 500 ng/day, 600 ng/day, 700 ng/day, 800 ng/day, 900 ng/day, 1 µg/day, 5 µg/day, 10 µg/day, 25 µg/day, 50 µg/day, 75 µg/day, 100 µg/day, 150 µg/day, 200 µg/day, 250 µg/day, 300 µg/day, 350 µg/day, 400 µg/day, 450 µg/day, 500 µg/day, 550 µg/day, 600 µg/day, 650 µg/day, 700 µg/day, 750 µg/day, 800 µg/day, 850 µg/day, 900 µg/day, 950 µg/day, 1 mg/day, 5 mg/day, 10 mg/day, 15 mg/day, 20 mg/day, 25 mg/day, 30 mg/day, 35 mg/day, 40 mg/day, 45 mg/day, 50 mg/day, 55 mg/day, 60 mg/day, 65 mg/day, 70 mg/day, 75 mg/day, 80 mg/day, 85 mg/day, 90 mg/day, 95 mg/day, 100 mg/day.

In one embodiment, a therapeutically effective amount of the peptide, composition, pharmaceutical composition or medicament according to the present invention is to be administered once a day, twice a day, three times a day or more.

In one embodiment, a therapeutically effective amount of the peptide, composition, pharmaceutical composition or medicament according to the present invention is to be administered every day, every two days, every three days, every four days, every five days, every six days.

In one embodiment, a therapeutically effective amount of the peptide, composition, pharmaceutical composition or medicament according to the present invention is to be administered every week, every two weeks, every three weeks.

In one embodiment, a therapeutically effective amount of the peptide, composition, pharmaceutical composition or medicament according to the present invention is to be administered every month, every two months, every three months, every four months, every five months, every six months.

In a preferred embodiment, a therapeutically effective amount of the peptide, composition, pharmaceutical composition or medicament according to the present invention is to be administered every 12 hours, every 24 hours, every 36 hours, every 48 hours, every 60 hours, every 72 hours, every 96 hours.

In a preferred embodiment, a therapeutically effective amount of the peptide, composition, pharmaceutical composition or medicament according to the present invention is to be administered every 60 hours.

In one embodiment, the peptide, composition, pharmaceutical composition or medicament according to the present invention is for acute administration. In one embodiment, the peptide, composition, pharmaceutical composition or medicament according to the present invention is for chronic administration.

In one embodiment, a therapeutically effective amount of the peptide, composition, pharmaceutical composition or medicament according to the present invention is to be administered for about 5 days, 7 days, 10 days, 14 days, 21 days, 28 days, 1 month, 2 months, 3 months, 6 months, 1 year or more.

In one embodiment, the peptide, composition, pharmaceutical composition or medicament according to the present invention is to be administered before, concomitantly with or after an additional therapeutic or diagnostic agent.

Suitable additional therapeutic or diagnostic agents include all those described hereinabove as therapeutic or diagnostic payloads of the peptide conjugates, including, but are not limited to, chemotherapeutic agents, targeted therapy agents, cytotoxic agents, antibiotics, antivirals, cell cycle-synchronizing agents, ligands for cellular receptor(s), immunomodulatory agents, pro-apoptotic agents, anti-angiogenic agents, cytokines, growth factors, antibodies or antigen-binding fragments thereof, hormones, coding or non-coding oligonucleotides, photodetectable labels, contrast agents, radiolabels, and the like.

The skilled artisan will readily understand that these therapeutic or diagnostic agents can, additionally or alternatively, be provided as unconjugated to the peptides according to the present invention.

Accordingly, the present invention also relates to a method of promoting or enhancing reparation of DNA lesions or damages induced by a chemotherapeutic agent or by a radiotherapy in a subject in need thereof, comprising co-administering to said subject (i) said chemotherapeutic agent or radiotherapy and (ii) a peptide according to the present invention, or the composition, the pharmaceutical composition or the medicament according to the present invention.

The present invention also relates to a peptide according to the present invention, or to the composition, the pharmaceutical composition or the medicament according to the present invention, for use in promoting or enhancing reparation of DNA lesions or damages induced by a chemotherapeutic agent or a radiotherapy co-administered to said subject.

In one embodiment, the peptide according to the present invention, or the composition, the pharmaceutical composition or the medicament according to the present invention, is administered or to be administered to the subject before, concomitantly with, or after administration of the chemotherapeutic agent or radiotherapy; preferably the peptide according to the present invention, or the composition, the pharmaceutical composition or the medicament according to the present invention, is administered or to be administered to the subject after administration of the chemotherapeutic agent or radiotherapy.

Examples of DNA lesions or damages include, but are not limited to, oxidized nucleobases, alkylated nucleobases, thymine-thymine dimers, small base adducts, single-strand breaks (SSBs), DNA helix-distorting base lesions, and stalled replication.

Suitable chemotherapeutic agents include all those described hereinabove as therapeutic or diagnostic payloads of the peptide conjugates.

The following items are also disclosed herein:

Item 1: an isolated peptide which specifically binds to human CD38 having SEQ ID NO: 3 and which comprises at most 25 contiguous amino acid residues from human CD31 having SEQ ID NO: 2 or a variant thereof.

Item 2: the isolated peptide of item 1, being any one of the peptides of Table 1.

Item 3: the isolated peptide of item 1 or 2, being a 2-D structured peptide, preferably cyclized and/or beta-turned.

Item 4: the isolated peptide of item 3, wherein the 2-D structured peptide is any one of the peptides of Table 1, wherein:
a) (i) a cysteine residue is inserted at both N-terminal and C-terminal extremities; and
(ii) any cysteine residue within the isolated peptide sequence is either protected or mutated to another amino acid residue, preferably to serine; and/or
b) a Pro-Gly or Asp-Gly dipeptide is inserted at a position which is central in said isolated peptide.

Item 5: the isolated peptide of any one of items 1 to 4, being selected from the group comprising SEQ ID NOs: 4 to 15, preferably SEQ ID NOs: 4 to 13, more preferably SEQ ID NOs: 6 to 10.

Item 6: the isolated peptide of any one of items 1 to 5, being fused to a payload being a therapeutic or diagnostic payload and/or a carrier payload.

Item 7: a nucleic acid encoding the isolated peptide of any one of items 1 to 6.

Item 8: An expression vector comprising the nucleic acid of item 7.

Item 9: a composition comprising at least one isolated peptide of any one of items 1 to 6.

Item 10: the isolated peptide of any one of items 1 to 6, for use in preventing and/or treating a disease selected from inflammatory diseases; autoimmune diseases; metabolic and endocrine diseases; neurodegenerative diseases; neuroinflammatory diseases; ocular diseases; age-related diseases; and cancer and metastasis; in a subject in need thereof.

Item 11: the isolated peptide for use of item 10, wherein said disease is selected from multiple sclerosis; rheumatoid arthritis; systemic lupus erythematosus; diabetes; obesity; non-alcoholic steatohepatitis; amyotrophic lateral sclerosis; Parkinson’s disease and related disorders; Alzheimer’s disease and related disorders; Huntington disease; age-related macular degeneration; and glaucoma.

Item 12: the isolated peptide of any one of items 1 to 6, for use in increasing the level of at least one anti-inflammatory cytokine in a subject in need thereof, in particular in the blood of said subject.

Item 13: the isolated peptide for use of item 12, wherein said anti-inflammatory cytokine is interleukin-10 (IL-10).

Item 14: the isolated peptide of any one of items 1 to 6, for use in lowering glucose levels in a subject in need thereof, in particular in the blood of said subject.

Also disclosed herein are CD31-derived peptides with SEQ ID NOs: 19 to 284.

### BRIEF DESCRIPTION OF THE DRAWINGS

FIG. 1 is a plot showing the neuroprotective effect of sCD31 against MPP⁺-induced dopaminergic (DA) cell death. DA (TH⁺) cell survival in midbrain cultures treated with MPP⁺ (3 µM) between 5 and 7 DIV exposed or not to sCD31 (0.01, 0.03, 0.1, 0.3, 1 µg/mL). Results are expressed in % of corresponding control cultures. $ P < 0.05 one-way ANOVA vs control treatment; # P < 0.05 one-way ANOVA vs MPP⁺ treatment.

FIG. 2 is a bar graph showing the neuroprotective effect of sCD31 against MPP⁺-induced DA cell death. DA (TH⁺) cell survival in midbrain cultures treated with MPP⁺ (3 µM) between 5 and 7 DIV exposed or not to sCD31 (1 µg/mL) in the presence or not of anti-CD31 antibody clone Moon-1 (1 µg/mL), of the antagonistic anti-CD38 antibody clone OKT10 (OKT, 1 µg/mL), or of the antagonistic anti-CD38 antibody clone AT13/5 (1 µg/mL). Results are expressed in % of corresponding control cultures. $ P < 0.05 one-way ANOVA vs control treatment; # P < 0.05 one-way ANOVA vs MPP⁺ treatment.

FIG. 3 is a bar graph showing the neuroprotective effect of agonistic anti-CD38 antibody clone HB7 against MPP⁺-induced DA cell death. DA (TH⁺) cell survival in midbrain cultures treated with MPP⁺ (3 µM) between 5 and 7 DIV exposed or not to agonistic anti-CD38 antibody (clone HB7, 1 µg/mL) in the presence or not of the antagonistic anti-CD38 antibody clone OKT10 (OKT, 1 µg/mL), or of the antagonistic anti-CD38 antibody clone AT13/5 (1 µg/mL). Results are expressed in % of corresponding control cultures. $ P < 0.05 one-way ANOVA vs control treatment; # P < 0.05 one-way ANOVA vs MPP⁺ treatment.

FIG. 4 is a bar graph showing the neuroprotective effect of sCD31 or agonistic anti-CD38 antibody clone HB7 against MPP⁺-induced DA cell death in the presence or absence of the tyrosine kinase inhibitor genistein. DA (TH⁺) cell survival in midbrain cultures treated with MPP⁺ (3 µM) between 5 and 7 DIV exposed or not to sCD31 (1 µg/mL) or agonistic anti-CD38 antibody (clone HB7, 1 µg/mL) in the presence or not of genistein (GENI, 10 nM), an inhibitor of tyrosine kinase. Results are expressed in % of corresponding control cultures. $ P < 0.05 one-way ANOVA vs control treatment; # P < 0.05 one-way ANOVA vs MPP⁺ treatment.

FIG. 5 is a bar graph showing the neuroprotective effect of sCD31 or agonistic anti-CD38 antibody clone HB7 against MPP⁺-induced DA cell death in the presence or absence of the lysosomal exocytosis inhibitor vacuolin-1 or endosidin2. DA (TH⁺) cell survival in midbrain cultures treated with MPP⁺ (3 µM) between 5 and 7 DIV exposed or not to sCD31 (1 µg/ml) or agonistic anti-CD38 antibody (clone HB7, 1 µg/ml) in the presence or not of vacuolin-1 (VAC, 10 µM), or endosidin2 (40 µM), two inhibitors of lysosomal maturation and exocytosis. Results are expressed in % of corresponding control cultures. $ P < 0.05 one-way ANOVA vs control treatment; # P < 0.05 one-way ANOVA vs MPP⁺ treatment.

FIG. 6 is a bar graph showing the effect of agonistic anti-CD38 HB7 antibody injected intravenously (15 mg/kg iv, two days before MPTP injections) in the in vivo MPTP mouse model on the number of DA (TH⁺) neurons in the substantia nigra pars compacta. Results are expressed in % of control (PBS-injected) mice. PBS group: n = 5; MPTP group: n = 6; MPTP + HB7 15 mg/kg iv group: n= 8. $ P < 0.05 one-way ANOVA vs control (PBS-injected) group. # P < 0.05 one-way ANOVA vs MPTP group.

FIG. 7 is a bar graph showing the effect of intravenous injection of agonistic anti-CD38 clone HB7 on IL-10 levels in mice. $ P < 0.05 one-way ANOVA vs control (PBS-injected) group.

FIG. 8 is a diagram depicting the process used to identify anti-CD38 antibodies that competes with CD31.

FIG. 9 is a bar graph showing the neuroprotective effect of new humanized anti-CD38 antibodies (clone A02, B06, B08, B09, C05, C06, C08, D03, D04, D06, D07, D10, E03, E05, E08, F07) identified by phage display or anti-CD38 antibody clone HB7 against MPP⁺-induced DA cell death. DA (TH⁺) cell survival in midbrain cultures treated with MPP⁺ (3 µM) between 5 and 7 DIV exposed or not to anti-CD38 antibodies (clone A02, B06, B08, B09, C05, C06, C08, D03, D04, D06, D07, D10, E03, E05, E08, F07; 0.5 µg/mL) or anti-CD38 antibody clone HB7 (0.5 µg/mL). Results are expressed in % of corresponding control cultures. # P < 0.05 one-way ANOVA vs MPP⁺ treatment.

FIG. 10 is a bar graph showing the neuroprotective effect of new humanized anti-CD38 antibodies (clones B08, C05, C06, D06 and D07) identified by phage display against MPP⁺-induced DA cell death in the presence or absence of the lysosomal exocytosis inhibitor vacuolin-1. DA (TH⁺) cell survival in midbrain cultures treated with MPP⁺ (3 µM) between 5 and 7 DIV exposed or not to anti-CD38 antibodies (clones B08, C05, C06, C08, D06 and D07; 1 µg/ml) in the presence or not of vacuolin-1 (VAC, 10 µM), an inhibitor of lysosomal exocytosis. Results are expressed in % of corresponding control cultures. $ P < 0.05 one-way ANOVA vs control treatment; # P < 0.05 one-way ANOVA vs MPP⁺ treatment.

FIG. 11 is a bar graph showing the effect of anti-CD38 antibodies (clone HB7, B08 or D06) injected intracerebrally (0.1 mg/kg icv, concomitantly with 6-OHDA icv injection) in the in vivo 6-OHDA mouse model on the number of DA (TH⁺) neurons in the substantia nigra pars compacta. Results are expressed in % of contralateral (non-lesioned) side. PBS group: n = 8 (PBS); 6-OHDA + PBS group: n = 8 (PBS); 6-OHDA + HB7 0.1 mg/kg icv group: n = 10 (HB7 icv 0.1 mg/kg); 6-OHDA + B08 0.1 mg/kg icv group: n = 7 (B8 icv 0.1 mg/kg); 6-OHDA + D06 0.1 mg/kg icv group: n= 7 (D6 icv 0.1 mg/kg). $ P < 0.05 one-way ANOVA vs control (PBS-injected) group. # P < 0.05 one-way ANOVA vs 6-OHDA group.

FIG. 12 is a bar graph showing the effect of anti-CD38 antibodies (clone B08 or Daratumumab, DARA) injected intravenously (15 mg/kg iv, one day before 6-OHDA icv injection) in the in vivo 6-OHDA mouse model on the number of DA (TH⁺) neurons in the substantia nigra pars compacta. Results are expressed in % of contralateral (non-lesioned) side. PBS group: n = 8 (PBS); 6-OHDA group: n = 8 (PBS); 6-OHDA + B08 15 mg/kg iv group: n = 10 (B8 iv 15 mg/kg); 6-OHDA + Daratumumab 15 mg/kg iv group: n = 10 (DARA iv 15 mg/kg). $ P < 0.05 one-way ANOVA vs control (PBS-injected) group. # P < 0.05 one-way ANOVA vs 6-OHDA group.

FIG. 13 is a bar graph showing the effect of the B08 anti-CD38 antibody injected intravenously (15 mg/kg iv, two days after 6-OHDA icv injection) in the in vivo 6-OHDA mouse model on the number of DA (TH⁺) neurons in the substantia nigra pars compacta. Results are expressed in % of contralateral (non-lesioned) side. PBS group: n = 12 (PBS); 6-OHDA + PBS group: n = 12 (PBS); 6-OHDA + B08 15 mg/kg iv group: n = 12 (B8 iv 15 mg/kg). $ P < 0.05 one-way ANOVA vs control (PBS-injected) group. # P < 0.05 one-way ANOVA vs 6-OHDA group.

FIG. 14 is a bar graph showing the effect of anti-CD38 antibodies (clone HB7, B08, C05, D06 or D07) on the release of the anti-inflammatory cytokine IL-10 using human peripheral blood mononuclear cells from 3 healthy donors (pooled results). $ P < 0.05 one-way ANOVA vs control (PBS-treated) group.

FIGS. 15A-B are a set of scheme and graph. PBS group: n = 10 (PBS); B08 15 mg/kg iv group: n= 10 (B8 15 mg/kg iv). * P < 0.05 two-way ANOVA vs PBS group. ** P < 0.01 two-way ANOVA vs PBS group. Clinical score is calculated as described in the material and method section.

FIG. 15A is a scheme representing the protocol.

FIG. 15B is a bar graph showing the effect of anti-CD38 antibody (clone B08) injected intravenously (15 mg/kg iv, 10 and 20 days after MOG injection) in the in vivo EAE mouse model on the clinical score.

FIGS. 16A-B are a set of schema and graph. No-DSS group: n = 5 (PBS; white bar); DSS + PBS group: n = 6 (PBS; black bar); DSS + DARA 15 mg/kg iv group: n = 7 (DARA iv 15 mg/kg; black bar); DSS + B08 15 mg/kg iv group: n = 6 (B8 iv 15 mg/kg; black bar). $ P < 0.05 one-way ANOVA vs no-DSS group. # P < 0.05 one-way ANOVA vs DSS group.

FIG. 16A is a scheme representing the protocol.

FIG. 16B is a bar graph showing the effect of anti-CD38 antibodies (clone B08 or Daratumumab, DARA) injected intravenously (15 mg/kg iv, injected at D0) in the in vivo DSS mouse model on colon length.

FIGS. 17A-D are a set of four graphs showing the intensity profiles recorded for His-tagged CD38 at 20 µg/mL and 9 µg/mL on the CD31 peptide arrays. Signal intensities are plotted as a line plot indicating the binding intensity on the y-axis and CD31 peptide position in the sequence (indicated by the amino acid number of the last residue of each peptide; numbering based on residues 28 to 601 of SEQ ID NO: 1) on the x-axis.

FIG. 17A shows the intensity profiles recorded for His-tagged CD38 on the linear peptide arrays (LIN15).

FIG. 17B shows the intensity profiles recorded for His-tagged CD38 on the conformational peptide arrays (LOOP7).

FIG. 17C shows the intensity profiles recorded for His-tagged CD38 on the conformational peptide arrays (LOOP15).

FIG. 17D shows the intensity profiles recorded for His-tagged CD38 on the conformational peptide arrays (BET).

FIG. 18 is a bar graph showing the effect of insulin (1 µM - second bar), sCD31 (SEQ ID NO: 2; 1 µM - third bar) or test compounds (SEQ ID NOs: 4-17; 1 µM) versus negative control (first bar), on glucose uptake expressed as relative percentage of 2-NBDG fluorescence using human Daudi cells.

FIG. 19 is a bar graph showing the effect of insulin (1 µM - second bar), sCD31 (SEQ ID NO: 2; 1 µM - third bar) or test compounds (SEQ ID NOs: 7 and 285; 1 µM) versus negative control (first bar), on glucose uptake expressed as relative percentage of 2-NBDG fluorescence using human Daudi cells.

FIG. 20 is a bar graph showing the effect of insulin (1 µM - second bar), sCD31 (SEQ ID NO: 2; 1 µM - third bar) or test compounds (SEQ ID NOs: 6 and 286-290; 1 µM) versus negative control (first bar), on glucose uptake expressed as relative percentage of 2-NBDG fluorescence using human Daudi cells.

FIG. 21 is a bar graph showing the effect of insulin (1 µM - second bar), sCD31 (SEQ ID NO: 2; 1 µM - third bar) or test compounds (SEQ ID NOs: 8 and 292; 1 µM) versus negative control (first bar), on glucose uptake expressed as relative percentage of 2-NBDG fluorescence using human Daudi cells.

FIG. 22 is a bar graph showing the effect of insulin (1 µM - second bar), sCD31 (SEQ ID NO: 2; 1 µM - third bar) or test compounds (SEQ ID NOs: 8 and 293-300; 1 µM) versus negative control (first bar), on glucose uptake expressed as relative percentage of 2-NBDG fluorescence using human Daudi cells.

FIG. 23 is a bar graph showing the effect of insulin (1 µM - second bar), sCD31 (SEQ ID NO: 2; 1 µM - third bar) or test compounds (SEQ ID NOs: 8, 293 and 301-304; 1 µM) versus negative control (first bar), on glucose uptake expressed as relative percentage of 2-NBDG fluorescence using human Daudi cells.

FIG. 24 is a bar graph showing the effect of insulin (1 µM - second bar), sCD31 (SEQ ID NO: 2; 1 µM - third bar) or test compounds (SEQ ID NOs: 8, 294 and 305-310; 1 µM) versus negative control (first bar), on glucose uptake expressed as relative percentage of 2-NBDG fluorescence using human Daudi cells.

FIG. 25 is a bar graph showing the effect of insulin (1 µM - second bar), sCD31 (SEQ ID NO: 2; 1 µM - third bar) or test compounds (SEQ ID NOs: 8, 296 and 311-315; 1 µM) versus negative control (first bar), on glucose uptake expressed as relative percentage of 2-NBDG fluorescence using human Daudi cells.

FIG. 26 is a bar graph showing the effect of insulin (1 µM - second bar), sCD31 (SEQ ID NO: 2; 1 µM - third bar) or test compounds (SEQ ID NOs: 8, 297 and 316-319; 1 µM) versus negative control (first bar), on glucose uptake expressed as relative percentage of 2-NBDG fluorescence using human Daudi cells.

FIG. 27 is a bar graph showing the effect of insulin (1 µM - second bar), sCD31 (SEQ ID NO: 2; 1 µM - third bar) or test compounds (SEQ ID NOs: 8 and 320; 1 µM) versus negative control (first bar), on glucose uptake expressed as relative percentage of 2-NBDG fluorescence using human Daudi cells.

FIG. 28 is a plot showing the dose response effect of sCD31 (SEQ ID NO: 2), SEQ ID NO: 6, SEQ ID NO: 9 and SEQ ID NO: 10 on glucose uptake assessed by 2-NBDG fluorescence using human Daudi cells.

FIGS. 29A-B are a set of two plots showing Biacore binding of SEQ ID NO: 6 and SEQ ID NO: 10 on human recombinant CD38 protein (SEQ ID NO: 3).

FIG. 29A shows Biacore binding of SEQ ID NO: 6 on human recombinant CD38 protein (SEQ ID NO: 3).

FIG. 29B shows Biacore binding of SEQ ID NO: 10 on human recombinant CD38 protein (SEQ ID NO: 3).

FIGS. 30A-D are a set of four bar graphs showing the effect of SEQ ID NO: 10 (300 µg/kg s.c. every day) in the HFD-induced obesity mouse model. * p < 0.05, *** p < 0.001, one-way ANOVA vs normal diet mice. n = 8 mice per group.

FIG. 30A shows the effect on daily food intake.

FIG. 30B shows the effect on weight increase.

FIG. 30C shows the effect on glycaemia (area under the curve) after performing a glucose challenge.

FIG. 30D shows the effect on insulin (area under the curve) after performing a glucose challenge.

FIG. 31 is a plot showing the effect of SEQ ID NO: 10 (300 µg/kg s.c. every day) on blood urea nitrogen levels in a folic acid-induced acute kidney injury mouse model. n = 4 per group. * p < 0.05, one-way ANOVA vs control mice.

FIGS. 32A-C are a set of one plot and two bar graphs showing the effect of SEQ ID NO: 10 (300 µg/kg s.c. every day) on blood glucose, insulin level and insulin resistance in an LPS-induced obese type 2 diabetic (T2DM) mouse model. n = 5 per group. *** p< 0.001, one-way ANOVA vs LPS-treated mice.

FIG. 32A shows the effect on blood glucose level.

FIG. 32B shows the effect on insulin level.

FIG. 32C shows the effect on insulin resistance expressed as HOMA-IR index (HOmeostasis Model Assessment of Insulin Resistance).

FIG. 33 is a bar graph showing the effect of SEQ ID NO: 6 and SEQ ID NO: 10 (300 µg/kg s.c. every day) in the in vivo DSS mouse model on colon length. n = 5 mice per group. *** p < 0.05; *** p < 0.001, one-way ANOVA vs DSS mice.

FIGS. 34A-B are a set of two bar graphs showing the effect of SEQ ID NO: 10 versus negative control (PBS), on glucose uptake in dog and cat PBMCs in vitro. *** p < 0.001, t-test.

FIG. 34A shows the effect on dog PBMCs.

FIG. 34B shows the effect on cat PBMCs.

### EXAMPLES

The present invention is further described in the following examples, but the technical scope of the present invention is not limited to these examples.

Examples 1-7 below correspond to Examples 1-7 of WO2020/152290.

### Materials and Methods (Examples 1-7)

### In Vitro Experiments

### Midbrain Cell Cultures

Animals were treated in accordance with the Guide for the Care and Use of Laboratory Animals (National Research Council, 1996), European Directive 86/609, and the guidelines of the local institutional animal care and use committee. Cultures were prepared from the ventral mesencephalon of gestational age 15.5 Wistar rat embryos (Janvier Breeding Center, Le Genest St Isle, France). Dissociated cells in suspension obtained by mechanical trituration of midbrain tissue pieces were seeded at a density of 1.2-1.5×10⁵ cells/cm² onto tissue culture supports precoated with 1 mg/mL polyethylenimine diluted in borate buffer pH 8.3 as described (Michel et al., 1997. J Neurochem. 69(4):1499-507). The cultures were then maintained in N5 medium supplemented with 5 mM glucose, 5 % horse serum, and 0.5 % fetal calf serum, except for the first 3 DIV, when the concentration of fetal calf serum was 2.5% to favor initial maturation of the cultures (Guerreiro et al., 2008. Mol Pharmacol. 74(4):980-9). They were fed daily by replacing 70% of the medium. Routinely, mesencephalic cultures were established on Nunc 24-well culture plates (Thermofischer Scientific, Rochester, NY). Note that these cultures contain tyrosine hydroxylase (TH)⁺ neurons that were exclusively dopaminergic (Traver et al., 2006. Mol Pharmacol. 70(1):30-40). TH⁺ neurons represented approximately 1-2% of the total number of neuronal cells present in these cultures. The evaluation of the survival of DA neurons was performed by counting cells immunopositive for TH as described (Toulorge et al., 2011. Faseb J. 25(8):2563-73).

### MPP+ Intoxication Model

Treatments with MPP⁺ were performed in cultures where the spontaneous death process was prevented by long-term exposure to depolarizing concentrations of K⁺ (30 mM), in the presence of the glutamate receptor antagonist MK801 (5 µM) to prevent unwanted excitotoxic insults as described previously (Douhou et al., 2001. J Neurochem. 78(1):163-74). Treatments with MPP⁺ and potential neuroprotective molecules were carried out between 5 and 7 DIV.

### Quantification of Neuronal Survival

The cultures were fixed for 12 min using 4 % formaldehyde in Dulbecco’s phosphate-buffered saline (PBS), then washed twice with PBS before an incubation step at 4° C. for 24 hours with the following antibodies. A monoclonal anti-TH antibody diluted 1/5000 (ImmunoStar, Inc., Hudson, WI) was used to assess the survival of DA neurons. Antibody was diluted in PBS containing 0.2 % Triton X-100. Detection of the primary antibodies was performed with an Alexa Fluor-488 conjugate of an anti-mouse IgG antibody.

### Human PBMCs Cell Culture

3 healthy volunteers (Donor 1: woman, 23 years old; Donor 2: man, 29 years old; Donor 3: woman, 20 years old) served as blood donors. Venous blood (5 mL) was collected into heparinized tubes and immediately processed. Briefly, blood was diluted 1/1 in pre-warmed RPMI 1640 medium. PBMCs were isolated using gradient centrifugation in Ficoll solution (Sigma; 300×g, 30 minutes). Isolated PBMCs were re-suspended in 1 volume of pre-warmed RPMI 1640 medium and centrifuged again (300×g, 30 minutes). PBMCs were re-suspended in RPMI 1640 medium supplemented with 10 % human serum (Sigma-Aldrich) at a concentration of 10⁷ cells per mL. Cells were seeded in 96 well microplate (100 µL per well). Test compounds (10 µg/mL), LPS (500 ng/mL) and interferon-γ 1B (100 ng/mL) were added to PBMCs culture immediately after plating. After 24 hours, cell culture supernatants were collected and frozen at - 80° C. for further processing.

### IL-10 ELISA Assay

IL-10 levels in cell culture supernatants were assayed using an ELISA human IL-10 kit (Ozyme®) according to the indications for use specified by the manufacturer.

### Statistical Analysis

Simple comparisons between two groups were performed with Student’s t test. Multiple comparisons against a single reference group were made by one-way analysis of variance followed by Dunnett’s test when possible. When all pairwise comparisons were required, the Student-Newman-Keuls test was used. S.E.M. values were derived from at least three independent experiments.

### In Vivo Experiments

### Animals

Animals were treated in accordance with the Guide for the Care and Use of Laboratory Animals (National Research Council, 1996), with the European Directive 2010/63/EU, and the guidelines of the local institutional animal care and use committee. For all studies, 8 to 10 weeks-old male C57B1/6 mice (Janvier, France) were used. They were maintained on a 12:12 hours light/dark cycle with lights on at 8 a.m. The room temperature was kept at 20° C., with free access to standard diet and tap water.

### 6-OHDA and MPTP Mouse Models

### Experimental Design

Two in vivo mouse models were used: the 6-OHDA mouse model and the MPTP mouse model. For the 6-OHDA mouse model, the intoxication protocol was based on the unilateral stereotaxic intrastriatal injection of 6-OHDA in the right striatum. Intracerebral or intravenous injections of treatments were made either the day before the surgical stereotaxic procedure, concomitantly with 6-OHDA during the surgical stereotaxic procedure, or two days after 6-OHDA injection. Animals were sacrificed 8 days after the surgical stereotaxic procedure.

For the MPTP mouse model, 4 injections of MPTP (20 mg/kg) were made. Intravenous injection of anti-CD38 clone HB7 antibody was done 2 days before MPTP injection. Mice were sacrificed 7 days after MPTP injections.

### Surgical Stereotaxic Procedure

For unilateral 6-OHDA mouse model, animals were anesthetized using Chloral hydrate (400 mg/kg, i.p.) and placed into a stereotactic frame adapted to mice. The injection was performed using a Hamilton syringe at the following coordinates: AP: + 0.85 cm, ML: ± 2 cm, DV: - 3.4 cm (corresponding to the atlas of Franklin and Paxinos, 1997). A total volume of 2.5 µL containing or not 5 µg of 6-OHDA diluted in PBS in the presence or the absence of test treatments was injected. The needle was left in place for 10 minutes after the injection before retraction.

### Tissue Preparation

Mice were sacrificed by cervical dislocation, and beheaded to collect brain and blood samples. The brain was fixed in a paraformaldehyde solution (4 %) during 1 day before being soaked in sucrose (30 %) during 2 days and then froze at -80° C. for further analysis.

### Brain Slicing, Immunofluorescence and Neurons Enumeration

Each brain was sliced using a freezing microtome at -30° C. The thickness of each slice was 20 µm. The slicing was done around the substantia nigra (80 slices from slice 51 to 63 according to Allen Mouse Brain).

Slices were then washed out in PBS and incubated with anti-Tyrosine Hydroxylase to stain dopaminergic neurons (Abcam®) during 2 days, at 4° C., with agitation. Slices were incubated with corresponding secondary antibody in the presence of DAPI (Sigma Aldrich®) to stain cell’s nuclei during 2 hours at room temperature, and mounted on gelatin-coated slides.

The slices were imaged using a Nikon TE2000 U equipped with a Hamamatsu ORCE-ER camera or with a Zeiss Axio Vert.A1 equipped with an axiocam 503 mono camera. Image analysis was done using Image J software or Zen (Zeiss®).

### IL-10 ELISA Assay

Blood samples collected in collection tube (Microvette® 500 EDTA-K3, SARSTEDT) were centrifuged at 3000 rotations per minute during 15 minutes. Supernatants (plasma) were collected and transferred into tubes and froze at -80° C. for further analysis. IL-10 levels in plasma samples were assayed using an ELISA IL-10 kit (BioLegend®) according to the indications for use specified by the manufacturer.

### EAE Mouse Model

### Experimental Design

Animals were anesthetized via inhalation with isoflurane (15 minutes). Mouse were immunized by subcutaneous injection of an emulsion containing 100 µg MOG peptide 35-55 in Complete Freund’s Adjuvant and 500 µg HKMT (heat-killed Mycobacterium tuberculosis) on day 0. Two hours later, animals received an intraperitoneal injection of 500 ng of pertussis toxin on day 0 and day 2. Test compounds were injected intravenously 10 and 20 days post-emulsion subcutaneous injection. Animals were sacrificed at day 28.

### Clinical Score Evaluation

After immunization, animals were daily monitored for weight and clinical symptoms. Clinical scores were graded according to the following scale: 0: no clinical signs; 0.5: partially limp tail; 1: paralyzed tail, normal gait; 1.5: paralyzed tail, uncoordinated movement, hind limb paresis; 2: one hind limb paralyzed or does not respond to pinch; 2.5: one hind limb paralyzed and weakness of the other; 3: two hind limbs paralyzed; 3.5: two hind limbs paralyzed and weakness of forelimbs; 4: moribund state.

### DSS Mouse Model

### Experimental Design

Experimental colitis was induced in mice by giving drinking water ad libitum containing 3 % (w/v) DSS for 5 days, while control mice received tap water only. Test compounds were intravenously administered (15 mg/kg) when DSS treatment began. Mice were sacrificed 9 days after the beginning of DSS treatment.

### Statistical Analysis

The statistical analyses and figures were made using the software Sigma Plot. Different tests may be performed depending on the data: One-way or two-way ANOVA and post hoc tests, if the conditions of application are respected.

### Example 1: Neuroprotection of Midbrain Dopaminergic Neurons by sCD31 Is Mediated Through Interaction With CD38

### Protection Against the Mitochondrial Neurotoxin MPP⁺

We report here that sCD31 is neuroprotective in a situation in which DA cell death was caused by mitochondrial poisoning with 1-methyl-4-phenylpyridinium (MPP⁺), the active metabolite of the DA neurotoxin 1-methyl-4-phenyl-1,2,3,6-tetrahydropyridine (MPTP).

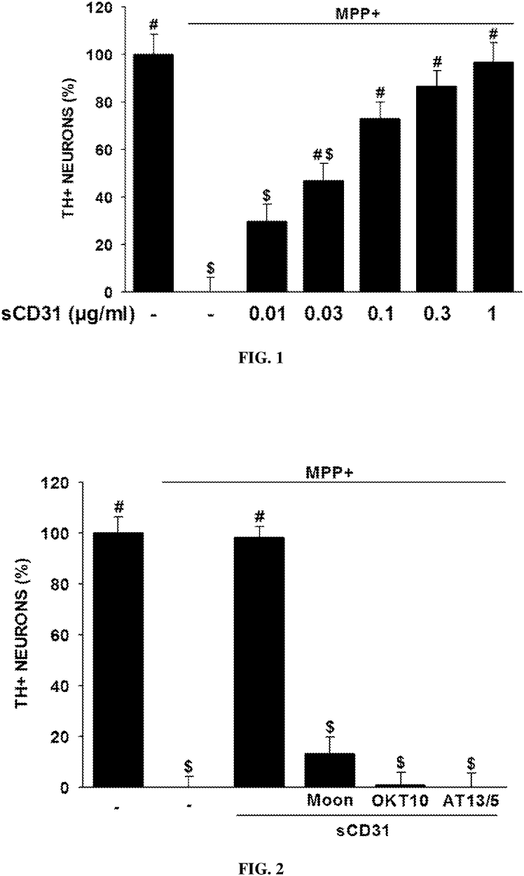

For this purpose, spontaneously occurring DA cell death was prevented by a treatment combining depolarizing concentrations of K⁺ (30 mM) and MK801 (5 µM), a glutamate receptor antagonist used to prevent unwanted excitotoxic insult. The cultures were then exposed to 3 µM MPP⁺ between 5 and 7 days in vitro (DIV) to achieve a loss of approximately 50% of DA neurons. When the cultures were exposed to murine sCD31 (having SEQ ID NO: 18) during the intoxication period, MPP⁺-induced DA cell loss was prevented by this compound (FIG. 1) with an EC₅₀ of 36 ng/mL.

Of note, these effects are true, direct neuroprotective effects, and are not due to a possible interference with CD38 localized on immune cells (as illustrated in Hara-Yokoyama et al., 2008. Int Immunopharmacol. 8(1):59-70) since there are no peripheral immune cells in this in vitro cell culture setting.

### The Neuroprotective Effect of sCD31 Is Antagonized in the Presence of anti-CD31 Antibody Clone Moon-1 or Antagonistic Anti-CD38 Antibodies (Clone OKT-10 or AT13/5)

CD31 is known to interact with 3 ligands: CD31 itself, CD38 and the αvβ₃ integrin (Kalinowska & Losy, 2006. Eur J Neurol. 13(12):1284-90). However, CD31 is not expressed in neuronal cells (Williams et al., 1996. J Neurosci Res. 45(6):747-57). Interaction of CD31 with CD38 was previously shown to be antagonized in the presence of the anti-CD31 antibody clone Moon-1 or by antagonistic anti-CD38 antibodies (Deaglio et al., 1996. J Immunol. 156(2):727-34; Deaglio et al., 1998. J Immunol. 160(1):395-402).

To test whether the neuroprotective effect of sCD31 is mediated through CD38, we studied the neuroprotective effect of sCD31 (1 µg/mL) in the presence or the absence of anti-CD31 antibody clone Moon-1 (1 µg/mL) or antagonistic anti-CD38 antibodies (clone OKT10 or AT13/5, 1 µg/mL) in the previously described MPP⁺ in vitro model.

We observed that the neuroprotective effect of sCD31 was antagonized in the presence of anti-CD31 antibody clone Moon-1 or of the antagonistic anti-CD38 antibodies clones OKT10 or AT13/5 (FIG. 2).

The neuroprotective effect of sCD31 is thus indeed mediated through the interaction with CD38.

### The Neuroprotective Effect of sCD31 Is Reproduced by Agonistic Anti-CD38 Antibody (Clone HB7)

Agonistic anti-CD38 antibodies are compounds that reproduce the biological effect of CD31 binding to CD38 (Deaglio et al., 1998. J Immunol. 160(1):395-402). Thus, like sCD31, agonistic anti-CD38 antibodies should also be neuroprotective.

We observed that an agonistic anti-CD38 antibody (clone HB7, 1 µg/mL) was able to protect DA neurons in the MPP⁺ in vitro model, and that this neuroprotective effect was antagonized in the presence of antagonistic anti-CD38 antibodies (clone OKT10 or AT13/5, 1 µg/mL) (FIG. 3).

### The Neuroprotective Effect of sCD31 or of Agonistic Anti-CD38 Antibodies Requires Tyrosine Phosphorylation/activation of Tyrosine Kinase

As previously mentioned herein, CD38 is a multifunctional molecule including a receptor-mediated function through internalization, a tyrosine phosphorylation-mediated function and an enzyme-mediated function. Previous research demonstrated that agonistic (clone HB7) or antagonistic (clone OKT10 or AT13/5) anti-CD38 antibodies failed to modulate CD38 enzymatic activity in vitro (Deckert et al., 2014. Clin Cancer Res. 20(17):4574-83). Consequently, we wanted to determine whether sCD31 or agonistic anti-CD38 antibodies required tyrosine phosphorylation to mediate its neuroprotective effect, by using genistein, an inhibitor of tyrosine kinase.

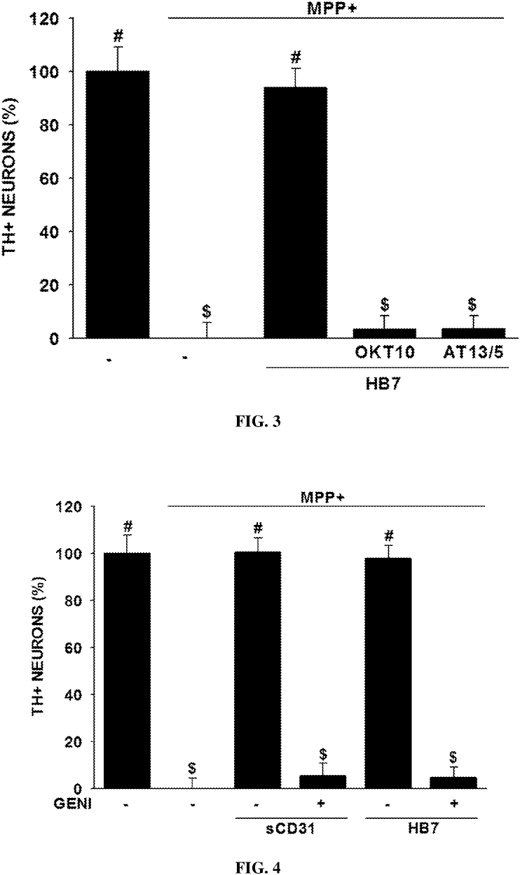

We observed that, in the MPP⁺ in vitro model, the neuroprotective effect of sCD31 (1 µg/mL) or of agonistic anti-CD38 antibodies (clone HB7, 1 µg/mL) was antagonized in the presence of the tyrosine kinase inhibitor genistein (GENI, 10 nM), suggesting that tyrosine phosphorylation is necessary to protect DA neurons (FIG. 4).

### The Neuroprotective Effect of sCD31 or of Agonistic Anti-CD38 Antibodies Is Mediated Through Tysosomal Exocytosis

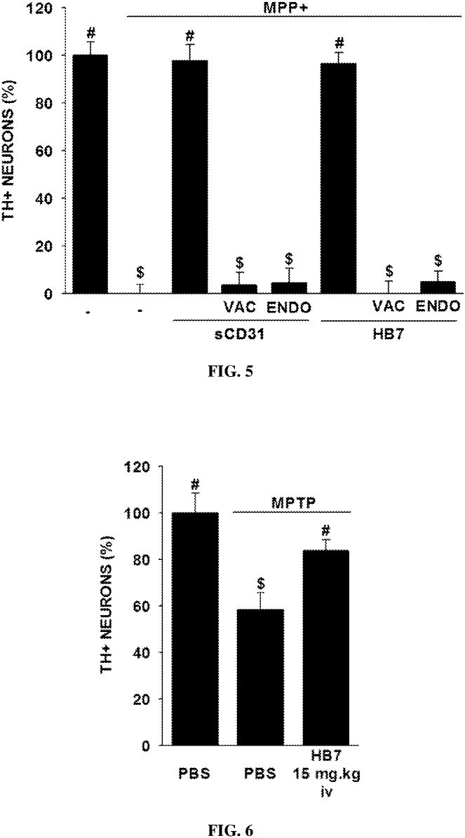

One may assume that sCD31 and agonistic anti-CD38 antibodies could both protect dopaminergic neurons by acting at the CD38 level but through a different mechanism of action. Ruling out this possibility, we observed (FIG. 5) that in the MPP⁺ in vitro model, the neuroprotective effect of sCD31 (1 µg/mL) or of agonistic anti-CD38 antibody (clone HB7, 1 µg/mL) were both antagonized in the presence of the inhibitors of lysosomal maturation and of Ca²⁺-dependent lysosomal exocytosis vacuolin-1 and endosidin2.

### Agonistic Anti-CD38 Antibody (clone HB7) Is Neuroprotective in Vivo

To determine whether agonistic anti-CD38 antibody (clone HB7) was also neuroprotective in vivo, we intravenously injected a 15 mg/kg dose of this antibody in a mouse model in which dopaminergic neurons of the substantia nigra pars compacta degenerate following repeated intraperitoneal injection of MPTP. MPTP has the ability to cross the blood brain barrier and is converted into MPP⁺ by the astrocytes. MPP⁺, which is an inhibitor of the mitochondrial complex I, is preferentially transported in dopaminergic neurons through the dopamine transporter, resulting in a specific cell death of dopaminergic population. This model has been extensively used as a test system for assessment of neuroprotective and neurorepair strategies (Dauer & Przedborski, 2003. Neuron. 39(6):889-909).

We observed that intravenous injection of agonistic anti-CD38 antibody (clone HB7) at a 15 mg/kg dose strongly protected dopaminergic neurons of the substantia nigra pars compacta from MPTP-induced neurodegeneration (FIG. 6).

### Conclusion

Altogether, the data presented in this example show that sCD31 is neuroprotective in vitro, an effect which is mediated through the interaction with CD38. This effect is antagonized in the presence of anti-CD31 antibodies or antagonistic anti-CD38 antibodies.

Additionally, it has been demonstrated that agonistic anti-CD38 antibodies, which mimic the binding of sCD31 to CD38, are also able to induce a neuroprotective effect, said effect being antagonized in the presence of antagonistic anti-CD38 antibodies.

Whether the neuroprotective effect is induced by sCD31 or by agonistic anti-CD38 antibodies, tyrosine phosphorylation/activation of tyrosine kinase has been shown to be necessary to induce said neuroprotective effect. Moreover, the resulting neuroprotective effect has been shown to be due to lysosomal maturation and Ca²⁺-dependent lysosomal exocytosis.

These data therefore suggest a therapeutic role of sCD31 for the treatment of neurodegenerative and neuroinflammatory diseases.

### Example 2: In Vivo Effect of an Agonistic Anti-CD38 Antibody on IL-10 Levels

To test whether the anti-inflammatory properties of agonistic anti-CD38 antibodies were also observe in vivo, we intravenously injected anti-CD38 antibody clone HB7 (15 mg/kg) to mice.

Eight days following injection, we observed a 6-fold increase in IL-10 levels (FIG. 7).

Altogether, the data presented in this example show that agonistic anti-CD38 antibodies induce a strong release of IL-10 in vivo, suggesting a role in immunoregulation and against inflammation. Indeed, IL-10 is widely known to downregulate the expression of Tₕ1 cytokines, MHC class II antigens, and co-stimulatory molecules on macrophages; to enhance B cell survival, proliferation, and antibody production; to block NF-κB activity; and to regulate the JAK-STAT signaling pathway.

Moreover, IL-10 has also been shown to be effective in treating cancer, in particular by inhibiting tumor metastasis (Sun et al., 2000. J Immunother. 23(2):208-14). Expression of IL-10 from transfected tumor cell lines in IL-10 transgenic mice (Groux et al., 1999. J Immunol. 162(3):1723-9) or dosing with IL-10 leads to control of primary tumor growth and decreased metastatic burden (Fujii et al., 2001. Blood. 98(7):2143-51; Berman et al., 1996. J Immunol. 157(1):231-8).

Given the consistent findings of Example 1 on the equal effects of sCD31 and agonistic anti-CD38 antibodies, these data therefore suggest a therapeutic role of sCD31 for the treatment of inflammatory diseases and cancer.

### Example 3: Identification of Agonistic Anti-CD38 Antibodies That Competes With CD31 by Phage Display

To generate new humanized antibodies that reproduce the biological effect of sCD31 on CD38, we used a phage display library of scFvs containing 1.5 billion sequences (Philibert et al., 2007. BMC Biotechnol. 7:81).

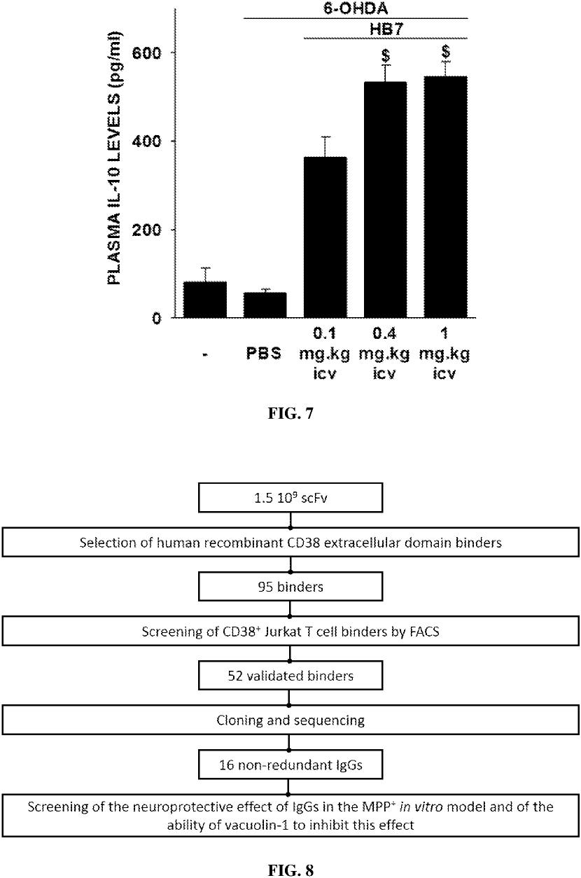

A first round of selection was done to identify scFvs that bind human CD38 extracellular domain (FIG. 8). Among 95 selected scFvs, 52 were confirmed to bind to CD38⁺ Jurkat T cells by FACS. These 52 validated binders were sequenced, leading to the identification of 16 non-redundant scFvs that bind CD38. These binders were cloned and the corresponding IgG were produced in CHO cells.

The 16 non-redundant IgGs were then tested (clones A02, B06, B08, B09, C05, C06, C08, D03, D04, D06, D07, D10, E03, E05, E08, F07), first in the MPP⁺ in vitro model (at 0.5 µg/mL for each clone), then by competition assay with the human extracellular domain of CD31 by Biacore on a panel of selected antibodies.

In the MPP⁺ in vitro model, several humanized anti-CD38 antibodies clones were found to be neuroprotective (FIG. 9). In particular, 4 clones (B08, C05, D06 and D10) shows a stronger neuroprotective effect than that of the reference agonistic anti-CD38 antibody clone HB7 (0.5 µg/mL).

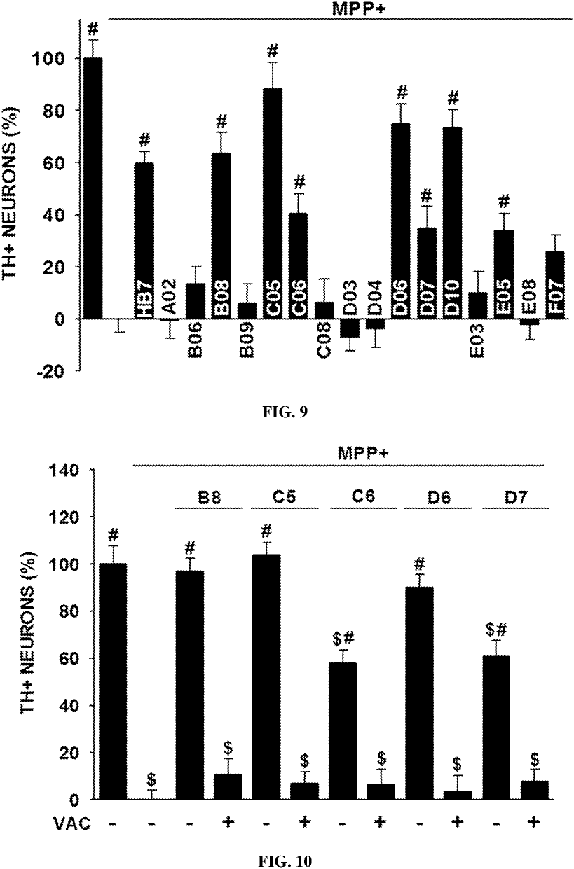

The neuroprotective effect of clones B08, C05, C06, D06 and D07 was antagonized in the presence of the lysosomal exocytosis inhibitors vacuolin-1 (VAC, 10 µM), suggesting that these antibodies reproduce the biological effect of sCD31 (FIG. 10).

### Example 4: Neuroprotective Properties of Proprietary Agonistic Anti-CD38 Antibodies in an In Vivo Neurodegenerative Model

We aimed at determining whether B08 or D06 anti-CD38 antibodies were neuroprotective in vivo against neurodegeneration induced by oxidative stress. Mouse were administered with 6-hydroxydopamine (6-OHDA) in the right striatum. Since 6-OHDA is an analogue of dopamine, it is transported in dopaminergic neurons through the dopamine transporter, resulting in a specific degeneration and loss of dopaminergic neurons of the right substantia nigra pars compacta. It has been extensively used as a test system for novel symptomatic agents and for assessment of neuroprotective and neurorepair strategies (Galindo et al., 2014. In Kostrzewa (Ed.), Handbook of neurotoxicity (1^{st} Ed., pp. 639-651). New York: Springer-Verlag). Since 6-OHDA is injected unilaterally in the right striatum, left striatum remains unaffected and each mouse is its own control.

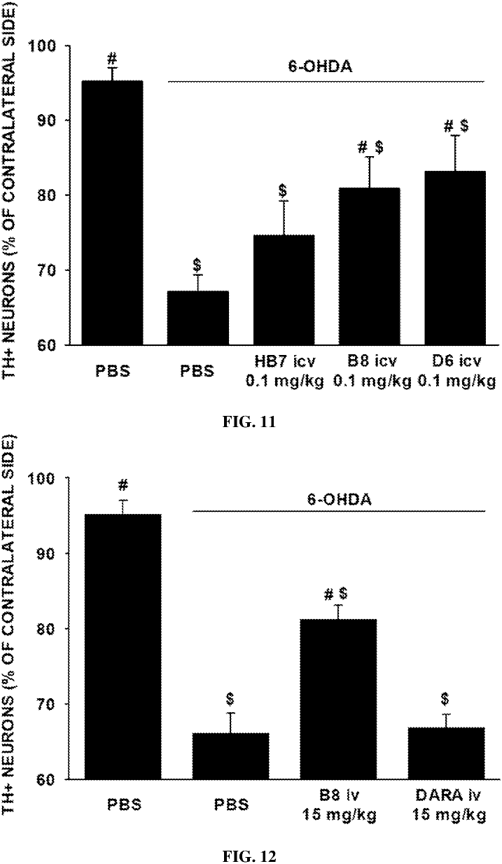

Mice were intracerebrally injected with 0.1 mg/kg of HB7, B08 and D06 anti-CD38 antibodies. We observed that at a concentration of 0.1 mg/kg injected intracerebrally (icv) concomitantly with 6-OHDA, B08 and D06 anti-CD38 antibodies statistically significantly protected dopaminergic neurons of the right substantia nigra pars compacta (FIG. 11). We also observed that HB7 anti-CD38 antibody did fail to statistically significantly protect dopaminergic neurons.

Of interest, regarding B08 anti-CD38 antibody, this neuroprotective effect was also observed when injected intravenously (iv, 15 mg/kg) one day before 6-OHDA icv injection (FIG. 12). Noticeably, the anti-CD38 antibody Daratumumab (DARA, 15 mg/kg iv) failed to demonstrate neuroprotective activity.

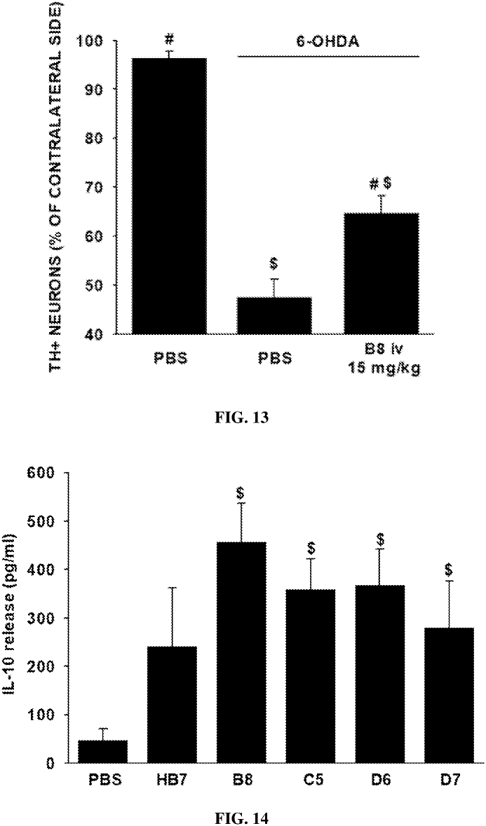

To understand whether B08 anti-CD38 antibody could also protect neurons when the neurodegenerative process is ongoing, we injected B08 anti-CD38 antibody (at 15 mg/kg iv) two days after 6-OHDA icv injection. We still observed strong neuroprotective activity of the B08 anti-CD38 antibody even after degeneration has started (FIG. 13). This demonstrates the powerful neuroprotective effect of B08 anti-CD38 antibody.

As a conclusion, the neuroprotective effect of B08 and D06 anti-CD38 antibodies were demonstrated to be of superior efficacy than that obtained with HB7 anti-CD38 antibody. Noticeably, this neuroprotective effect is still observed when the compound is injected intravenously when the neurodegenerative process is ongoing.

Given the consistent findings of Example 1 on the equal effects of sCD31 and agonistic anti-CD38 antibodies, these data therefore suggest a therapeutic role of sCD31 for the treatment of neurodegenerative diseases.

### Example 5: Anti-inflammatory Properties of Proprietary Agonistic Anti-CD38 Antibodies In Vitro

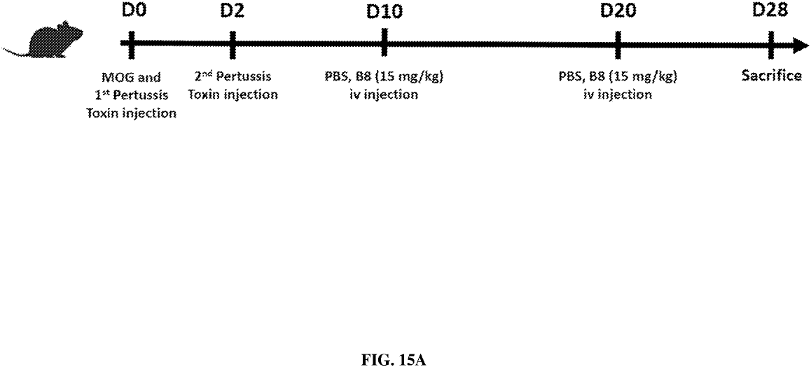

To determine whether agonistic anti-CD38 antibodies (clone B08, C05, D06 and D07) possess anti-inflammatory properties, we assessed their effect on the release of the anti-inflammatory cytokine IL-10 in vitro using human peripheral blood mononuclear cells (PBMCs) from 3 healthy donors. We observed that anti-CD38 antibodies (clone B08, C05, D06 and D07) all statistically significantly increased IL-10 release, thus demonstrating anti-inflammatory properties (FIG. 14). Of importance, HB7 failed to statistically significantly increase IL-10 release in culture human PBMCs in this assay, demonstrating the higher anti-inflammatory effect of the B08, C05, D06 and D07 anti-CD38 antibodies over the HB7 anti-CD38 antibody.

As a conclusion, agonistic anti-CD38 antibodies, as represented by clones B08, C05, D06 and D07, increase IL-10 release from PBMCs in vitro. In addition, because IL-10 is involved in the general mechanism of inflammation and has been reported in numerous auto-immune diseases, agonistic anti-CD38 antibodies, as represented by clones B08, C05, D06 and D07, provide a new strategy to treat and/or prevent inflammatory and/or auto-immune diseases.

Given the consistent findings of Example 1 on the equal effects of sCD31 and agonistic anti-CD38 antibodies, these data therefore suggest a therapeutic role of sCD31 for the treatment of inflammatory diseases.

### Example 6: Neuroprotective Effect of Proprietary Agonistic Anti-CD38 Antibody in an Experimental Autoimmune Encephalomyelitis (eae) Mouse Model of Multiple Sclerosis (ms), an Auto-Immune, Neuro-Inflammatory Disease

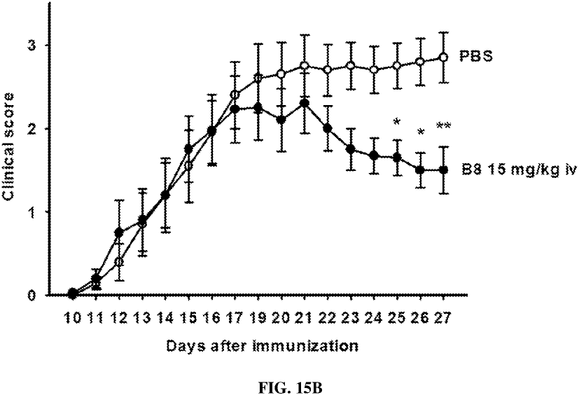

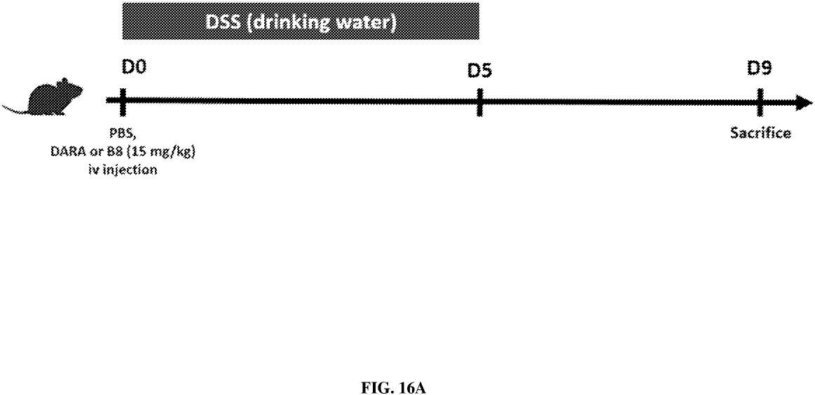

Multiple sclerosis (MS) is a progressive inflammatory and demyelinating disease of the human central nervous system (CNS) in which an important role for the immune system in the pathogenesis of the disease is suspected (Lassmann, 2008. J Neurol Sci. 274(1-2):45-7). EAE is an induced disease in mice that affects the central nervous system and constitutes a model for human MS. After a subcutaneous injection of an emulsion with a Complete Freund’s Adjuvant (CFA) mixed with oligodendrocyte membrane proteins like myelin oligodendrocyte glycoprotein (MOG), an immune response starts in the periphery and, within 2 weeks, causes inflammation in the CNS. In the same time, we can observe, a progressive ascending paralysis (tail to hindlimb to forelimb). To determine whether B08 anti-CD38 antibody could have beneficial effect in this disease, we injected this antibody (15 mg/kg at symptom onset (10 days after MOG injection) and at day 20). We observed that B08 anti-CD38 antibody significantly reduced the clinical score compared with PBS-injected mice (FIG. 15).

As a conclusion, agonistic anti-CD38 antibodies (clone B08) ameliorate the clinical score in the EAE mouse model of the auto-immune, neuro-inflammatory disease MS when injected intravenously at symptom onset.

Given the consistent findings of Example 1 on the equal effects of sCD31 and agonistic anti-CD38 antibodies, these data therefore suggest a therapeutic role of sCD31 for the treatment of auto-immune, neuro-inflammatory diseases.

### Example 7: Anti-inflammatory Properties of a Proprietary Agonistic Anti-CD38 Antibody In Vivo, in a Dextran Sulfate Sodium (DSS)-induced Colitis Mouse Model

To demonstrate the effect of B08 agonistic anti-CD38 antibody in a purely inflammatory model, we decided to test its effects in the Dextran Sulfate Sodium (DSS)-induced colitis mouse model. Administration of DSS causes human ulcerative colitis-like pathologies due to its toxicity to colonic epithelial cells, which results in compromised mucosal barrier function. Clinical observations similar to human pathologies, such as weight loss, diarrhea and occult blood in stool, are commonly observed in the DSS model. Moreover, diminution of the colon’s length is characteristic of this model. Importantly, studies suggest that DSS colitis mostly involves activation of lymphocytes, neutrophils and macrophages (Eichele and Kharbanda, 2017. World J Gastroenterol. 23(33):6016-29).

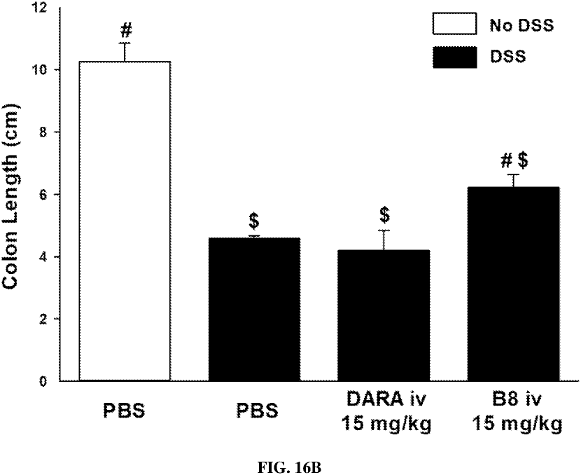

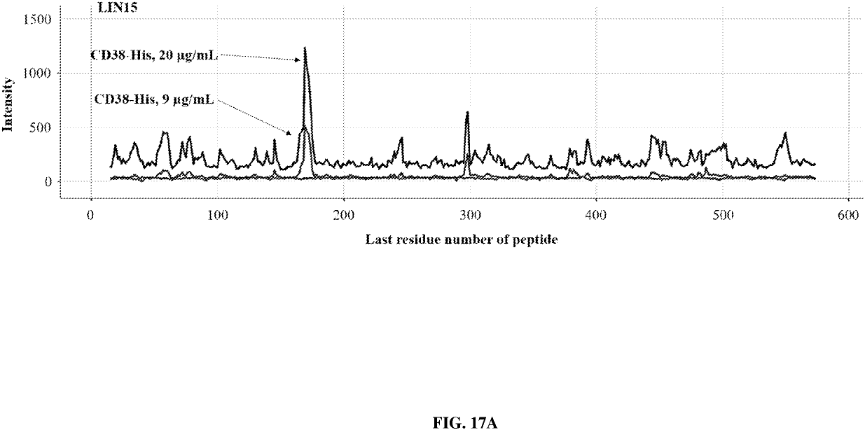

We observed that administration of B08 anti-DC38 antibody (15 mg/kg, iv) statistically significantly repressed reduction of the diminution of the colon’s length (FIG. 16).

As a conclusion, agonistic anti-CD38 antibody (clone B08) increases colon length in the DSS mouse model of inflammatory bowel disease.

Given the consistent findings of Example 1 on the equal effects of sCD31 and agonistic anti-CD38 antibodies, these data therefore suggest a therapeutic role of sCD31 for the treatment of inflammatory diseases.

### Example 8: Identification of CD31 Peptides Binding to CD38

The study was conducted at Pepscan Presto BV (Lelystad, The Netherlands). The aim was to identify epitopes of sCD31 specifically binding to CD38.

The concept of mapping linear epitopes using libraries of overlapping synthetic peptides is described in Geysen et al., 1984. Proc Natl Acad Sci USA. 81(13):3998-4002. Briefly, a library of linear, overlapping peptides of sCD31 were synthetized on a solid support as described further below. Considering that a majority of biomolecules recognize conformational or discontinuous epitopes, CLIPS (Chemically Linked Peptides on Scaffolds) technology was used to structurally fix peptides of sCD31 into defined 2-D structures, as described in Timmerman et al., 2007. J Mol Recognit. 20(5):283-299. Briefly, the CLIPS reaction takes place between bromo groups of the CLIPS scaffold and thiol sidechains of cysteines introduced into peptide constructs. Using this chemistry, native protein sequences are transformed into CLIPS constructs with a range of different structures including single mP2 loops, stabilized beta sheets, alpha helices, T3 double loops, etc.

### Material and Methods

### Design of the Peptides

Different sets of sCD31 peptides were synthesized according to the following designs. Note that actual order of peptides on mini-cards in some was randomized.
Set #1
   Mimic type: linear
   Label: LIN15
   Linear peptides of 15 consecutive amino acid residues in length, derived from the target sequence of sCD31 with an offset of 1.5 residues.
Set #2
   Mimic type: single loop (mP2 CLIPS)
   Label: LOOP7
   Constrained peptides of 9 amino acid residues in length. On positions 2-8 are incorporated 7 consecutive amino acid residues derived from the target sequence of sCD31 with an offset of 1.3 residues. Cys residues were inserted on positions 1 and 9 and joined by mP2 CLIPS in order to create a loop mimic. Native Cys residues, if present, were replaced by Ser residues.
Set #3
   Mimic type: single loop, (mP2 CLIPS)
   Label: LOOP15
   Constrained peptides of 17 amino acid residues in length. On positions 2-16 are incorporated 15 consecutive amino acid residues derived from the target sequence of sCD31 with an offset of 1.2 residues. Cys residues were inserted on positions 1 and 17 and joined by mP2 CLIPS in order to create a loop mimic. Native Cys residues, if present, were replaced by Ser residues.
Set #4
   Mimic type: β-turn peptide mimics (mP2 CLIPS)
   Label: BET
   β-turn peptide mimics of 22 amino acid residues in length. On positions 2-21 are incorporated 20 consecutive amino acid residues derived from the target sequence of sCD31 with an offset of 1.3 residues. Residues on positions 11 and 12 are replaced by a Pro-Gly motif in order to induce the β-turn formation. Cys residues were inserted on positions 1 and 22 and joined by mP2 CLIPS in order to stabilize the mimic. Native Cys residues, if present, were replaced by Ser residues.

### Peptide Synthesis

To reconstruct epitopes of sCD31, a library of peptide-based peptide mimics was synthesized using Fmoc-based solid-phase peptide synthesis. An amino functionalized polypropylene support was obtained by grafting with a hydrophilic polymer formulation, followed by reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups using trifluoroacetic acid (TFA).

Standard Fmoc-peptide synthesis was used to synthesize peptides on the amino-functionalized solid support by custom modified JANUS liquid handling stations (Perkin Elmer).

Synthesis of structural mimics was done using CLIPS technology. CLIPS technology allows to structure peptides into single loops, double loops, triple loops, sheet-like folds, helix-like folds, and combinations thereof. CLIPS templates are coupled to cysteine residues. The side-chains of multiple cysteines in the peptides are coupled to one or two CLIPS templates. For example, a 0.5 mM solution of the P2 CLIPS (2,6-bis(bromomethyl)pyridine) is dissolved in ammonium bicarbonate (20 mM, pH 7.8)/acetonitrile (1:3, v/v). This solution is added onto the peptide arrays. The CLIPS template binds to side-chains of two cysteines as present in the solid-phase bound peptides of the peptide-arrays (455 wells plate with 3-µL wells). The peptide arrays are gently shaken in the solution for 30 to 60 minutes while completely covered in solution.

Finally, the peptide arrays are washed extensively with excess of H₂O and sonicated in disrupt-buffer containing 1% SDS / 0.1 % 2,2′-(ethylenedioxy)diethanethiol in PBS (pH 7.2) at 70° C. for 30 minutes, followed by sonication in H₂O for another 45 minutes.

The T3 CLIPS carrying peptides were made in a similar way but now with three cysteines.

### ELISA Screening

The binding of the sample (recombinant His-tagged CD38; 9 µg/mL and 20 µg/mL) to each of the synthesized peptides was tested in a pepscan-based ELISA. The peptide arrays were incubated with primary sample solution (overnight at 4° C.). After washing, the peptide arrays were incubated with a 1/1000 dilution of an appropriate antibody peroxidase conjugate (rabbit anti-mouse IgG(H+L) HRP conjugate, Southern Biotech, Cat. No 6175-05; anti-His tag HRP, Novagen, Cat. No 70796-3) for 1 hour at 25° C.

After washing, the peroxidase substrate 2,2′-azino-di-3-ethylbenzthiazoline sulfonate (ABTS) and 20 µL/mL of 3 % H₂O₂ were added.

After one hour, the color development was measured. The color development was quantified with a charge coupled device (CCD) camera and an image processing system. The values obtained from the CCD camera range from 0 to 3000 mAU, similar to a standard 96-well plate ELISA-reader.

### Results

To extract the CD31 epitope(s) responsible for CD38 binding, initial experiments were carried out using recombinant CD38 fused to a mouse IgG2a Fc tail for detection. Unfortunately, results were ambiguous due to considerable overlap with a mouse IgG2a isotype control. While CD38 binding may occur, this was possibly obscured by the Fc binding.

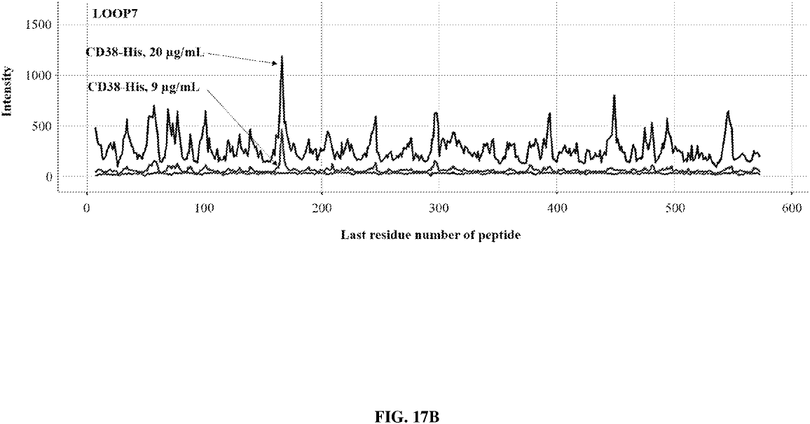

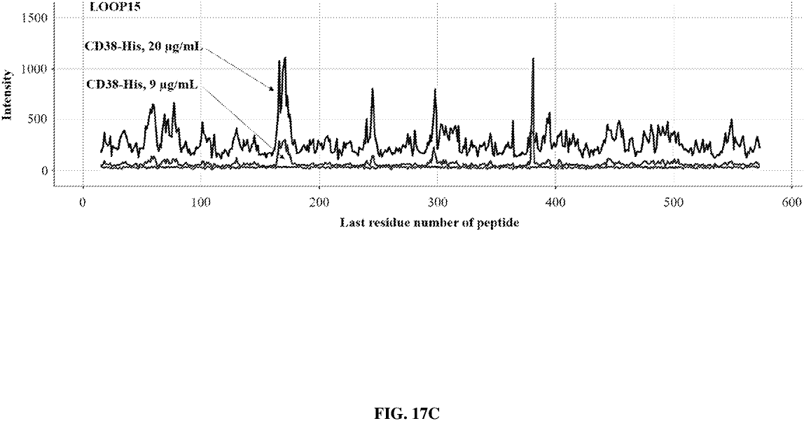

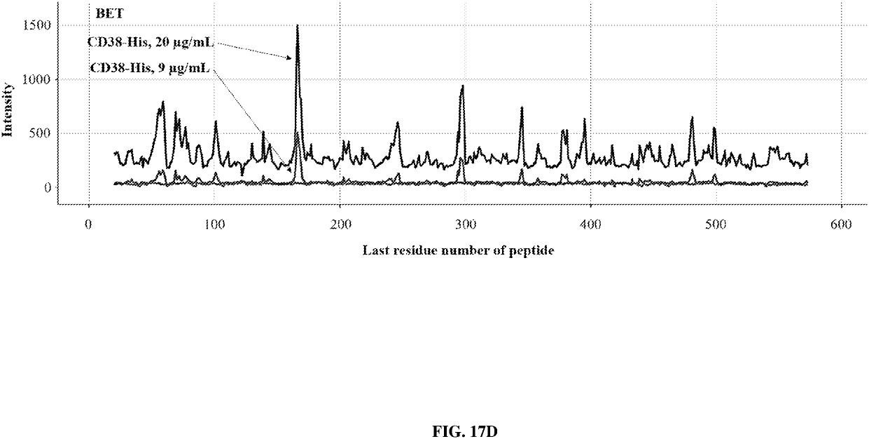

Therefore, current experiments were carried out with a recombinant His-tagged CD38. As no Fc tag was present, it is expected that the main interaction(s) would occur through the CD38 protein. Several binding peaks were found with linear and conformational mapping. In linear mapping (LIN15), we observe one main peak and several smaller peaks (FIG. 17A). Similar peaks were observed in the conformational array (LOOP7, FIG. 17B; LOOP15, FIG. 17C; BET, FIG. 17D).

A blank screening without sample but still using anti-His HRP for detection, did not show any binding. This suggests that all binding peaks that were observed are directly related to the His-tagged CD38 sample binding to CD31 peptides.

For determination of the binding site, the sample with the highest signal was used. The sequences with overlapping peptides are listed in Table 4.

Peaks were selected if the top peptides are at least 3 times the 20^{th} percentile value of the mimic type (in bold). Core binding sequences (underlined) are based on common sequences in overlapping peptides (overlapping sequences within 30 % of the top peptide intensity in the peak).

TABLE 4

list of sCD31 epitope candidates (for each of the four sets LIN15, LOOP7, LOOP15 and BET) found for His-tagged CD38.

| Mimic type | SEQ ID NO: 1 residues | | Epitope candidate | Value | SEQ ID NO: |
|---|---|---|---|---|---|
| | Start | End | | | |
| LIN15 | 68 | 82 | KPQHQMLFYKDDVLF | 355 | 30 |
| | 70 | 84 | QHQMLFYKDDVLFYN | 460 | 32 |
| | 71 | 85 | HQMLFYKDDVLFYNI | 441 | 33 |
| | 73 | 87 | MLFYKDDVLFYNISS | 451 | 36 |
| | 74 | 88 | LFYKDDVLFYNISSM | 383 | 38 |
| | 178 | 192 | KREKNSRDQNFVILE | 440 | 74 |
| | 179 | 193 | REKNSRDQNFVILEF | 453 | 75 |
| | 181 | 195 | KNSRDQNFVILEFPV | 502 | 77 |
| | 182 | 196 | NSRDQNFVILEFPVE | 1230 | 6 |
| | 184 | 198 | RDQNFVILEFPVEEQ | 1021 | 78 |
| | 185 | 199 | DQNFVILEFPVEEQD | 963 | 80 |
| | 187 | 201 | NFVILEFPVEEQDRV | 631 | 83 |
| | 308 | 322 | SSRISKVSSIVVNIT | 168 | 100 |
| | 310 | 324 | RISKVSSIVVNITEL | 572 | 101 |
| | 311 | 325 | ISKVSSIVVNITELF | 642 | 102 |
| | 313 | 327 | KVSSIVVNITELFSK | 155 | 104 |
| ^{∗∗} | 457 | 471 | NSNDPAVFKDNPTED | 421 | 120 |
| | 458 | 472 | SNDPAVFKDNPTEDV | 410 | 121 |
| | 460 | 474 | DPAVFKDNPTEDVEY | 402 | 122 |
| | 461 | 475 | PAVFKDNPTEDVEYQ | 333 | 123 |
| | 463 | 477 | VFKDNPTEDVEYQCV | 303 | 124 |
| | 559 | 573 | KQKASKEQEGEYYCT | 337 | 168 |
| | 560 | 574 | QKASKEQEGEYYCTA | 316 | 169 |
| | 562 | 576 | ASKEQEGEYYCTAFN | 400 | 170 |
| | 563 | 577 | SKEQEGEYYCTAFNR | 454 | 171 |
| | 565 | 579 | EQEGEYYCTAFNRAN | 340 | 172 |
| LOOP7 | 52 | 58 | CNLTLQSFC | 370 | 173 |
| | 54 | 60 | CTLQSFADC | 427 | 174 |
| | 55 | 61 | CLQSFADVC | 561 | 175 |
| | 56 | 62 | CQSFADVSC | 413 | 176 |
| | 57 | 63 | CSFADVSTC | 390 | 177 |
| | 73 | 79 | CMLFYKDDC | 542 | 178 |
| | 74 | 80 | CLFYKDDVC | 604 | 179 |
| | 76 | 82 | CYKDDVLFC | 585 | 180 |
| | 77 | 83 | CKDDVLFYC | 579 | 181 |
| | 78 | 84 | CDDVLFYNC | 702 | 182 |
| | 80 | 86 | CVLFYNISC | 502 | 183 |
| | 89 | 95 | CKSTESYFC | 339 | 184 |
| | 90 | 96 | CSTESYFIC | 668 | 5 |
| | 91 | 97 | CTESYFIPC | 549 | 185 |
| | 93 | 99 | CSYFIPEVC | 400 | 186 |
| | 94 | 100 | CYFIPEVRC | 523 | 187 |
| | 95 | 101 | CFIPEVRIC | 414 | 188 |
| | 98 | 104 | CEVRIYDSC | 642 | 189 |
| | 99 | 105 | CVRIYDSGC | 479 | 190 |
| | 100 | 106 | CRIYDSGTC | 395 | 191 |
| | 120 | 126 | CTAEYQVLC | 492 | 192 |
| | 121 | 127 | CAEYQVLVC | 508 | 193 |
| | 122 | 128 | CEYOVLVEC | 651 | 194 |
| | 185 | 191 | CDQNFVILC | 628 | 195 |
| | 186 | 192 | CQNFVILEC | 942 | 196 |
| | 187 | 193 | CNFVILEFC | 1186 | 291 |
| | 189 | 195 | CVILEFPVC | 527 | 197 |
| | 190 | 196 | CILEFPVEC | 537 | 198 |
| | 263 | 269 | CLAQEFPEC | 412 | 199 |
| | 264 | 270 | CAQEFPEIC | 429 | 200 |
| | 265 | 271 | CQEFPEIIC | 476 | 201 |
| | 267 | 273 | CFPEIIIQC | 596 | 202 |
| | 315 | 321 | CSSIVVNIC | 364 | 203 |
| | 316 | 322 | CSIVVNITC | 387 | 204 |
| | 317 | 323 | CIVVNITEC | 630 | 205 |
| | 319 | 325 | CVNITELFC | 625 | 206 |
| | 320 | 326 | CNITELFSC | 512 | 207 |
| | 412 | 418 | CRISYDAQC | 287 | 208 |
| | 414 | 420 | CSYDAQFEC | 570 | 209 |
| | 415 | 421 | CYDAQFEVC | 628 | 210 |
| | 416 | 422 | CDAQFEVIC | 429 | 211 |
| | 467 | 473 | CNPTEDVEC | 402 | 212 |
| | 468 | 474 | CPTEDVEYC | 407 | 213 |
| | 470 | 476 | CEDVEYQSC | 804 | 167 |
| | 471 | 477 | CDVEYQSVC | 556 | 214 |
| | 501 | 507 | CDFVQISIC | 378 | 215 |
| | 502 | 508 | CEVQISILC | 529 | 216 |
| | 503 | 509 | CVQISILSC | 416 | 217 |
| | 514 | 520 | CESGEDIVC | 420 | 218 |
| | 515 | 521 | CSGEDIVLC | 573 | 219 |
| | 516 | 522 | CGEDIVLQC | 442 | 220 |
| | 518 | 524 | CDIVLQSAC | 389 | 221 |
| | 564 | 570 | CKEQEGEYC | 385 | 222 |
| | 566 | 572 | CQEGEYYSC | 619 | 223 |
| | 567 | 573 | CEGEYYSTC | 650 | 224 |
| | 568 | 574 | CGEYYSTAC | 552 | 225 |
| | 570 | 576 | CYYSTAFNC | 477 | 226 |
| LOOP15 | 66 | 80 | CHVKPQHQMLFYKDDVC | 431 | 227 |
| | 67 | 81 | CVKPQHQMLFYKDDVLC | 411 | 228 |
| | 68 | 82 | CKPQHQMLFYKDDVLFC | 467 | 229 |
| | 70 | 84 | CQHQMLFYKDDVLFYNC | 600 | 230 |
| | 71 | 85 | CHQMLFYKDDVLFYNIC | 564 | 231 |
| | 72 | 86 | CQMLFYKDDVLFYNISC | 646 | 232 |
| | 73 | 87 | CMLFYKDDVLFYNISSC | 630 | 233 |
| | 74 | 88 | CLFYKDDVLFYNISSMC | 497 | 234 |
| | 80 | 94 | CVLFYNISSMKSTESYC | 371 | 235 |
| | 82 | 96 | CFYNISSMKSTESYFIC | 552 | 236 |
| | 83 | 97 | CYNISSMKSTESYFIPC | 370 | 237 |
| | 84 | 98 | CNISSMKSTESYFIPEC | 478 | 238 |
| | 85 | 99 | CISSMKSTESYFIPEVC | 499 | 239 |
| | 86 | 100 | CSSMKSTESYFIPEVRC | 343 | 240 |
| | 88 | 102 | CMKSTESYFIPEVRIYC | 325 | 241 |
| | 89 | 103 | CKSTESYFIPEVRIYDC | 528 | 242 |
| | 90 | 104 | CSTESYFIPEVRIYDSC | 660 | 243 |
| | 91 | 105 | CTESYFIPEVRIYDSGC | 465 | 244 |
| | 92 | 106 | CESYFIPEVRIYDSGTC | 373 | 245 |
| | 94 | 108 | CYFIPEVRIYDSGTYKC | 416 | 246 |
| | 178 | 192 | CKREKNSRDQNFVILEC | 516 | 247 |
| | 179 | 193 | CREKNSRDQNFVILEFC | 1073 | 248 |
| | 180 | 194 | CEKNSRDQNFVILEFPC | 566 | 249 |
| | 181 | 195 | CKNSRDQNFVILEFPVC | 624 | 250 |
| | 182 | 196 | CNSRDQNFVILEFPVEC | 965 | 164 |
| | 184 | 198 | CRDQNFVILEFPVEEQC | 1110 | 9 |
| | 185 | 199 | CDQNFVILEFPVEEQDC | 634 | 251 |
| | 186 | 200 | CQNFVILEFPVEEQDRC | 729 | 252 |
| | 187 | 201 | CNFVILEFPVEEQDRVC | 524 | 253 |
| | 188 | 202 | CFVILEFPVEEQDRVLC | 550 | 254 |
| | 256 | 270 | CSTIQVTHLAQEFPEIC | 320 | 255 |
| | 257 | 271 | CTIQVTHLAQEFPEIIC | 504 | 256 |
| | 258 | 272 | CTQVTHLAQEFPEIIIC | 797 | 257 |
| | 259 | 273 | CQVTHLAQEFPEIIIQC | 617 | 258 |
| | 260 | 274 | CVTHLAQEFPEIIIQKC | 343 | 259 |
| | 308 | 322 | CSSRISKVSSIVVNITC | 287 | 260 |
| | 310 | 324 | CRISKVSSIVVNITELC | 567 | 261 |
| | 311 | 325 | CISKVSSIVVNITELFC | 795 | 262 |
| | 312 | 326 | CSKVSSIVVNITELFSC | 593 | 263 |
| | 313 | 327 | CKVSSIVVNITELFSKC | 211 | 264 |
| BET | 63 | 82 | CTTSHVKPQHPGLFYKDDVLFC | 679 | 265 |
| | 64 | 83 | CTSHVKPQHQPGFYKDDVLFYC | 725 | 266 |
| | 65 | 84 | CSHVKPQHQMPGYKDDVLFYNC | 662 | 267 |
| | 67 | 86 | CVKPQHQMLFPGDDVLFYNISC | 792 | 268 |
| | 68 | 87 | CKPQHQMLFYPGDVLFYNISSC | 641 | 269 |
| | 76 | 95 | CYKDDVLFYNPGSMKSTESYFC | 378 | 270 |
| | 77 | 96 | CKDDVLFYNIPGMKSTESYFIC | 695 | 271 |
| | 78 | 97 | CDDVLFYNISPGKSTESYFIPC | 524 | 272 |
| | 80 | 99 | CVLFYNISSMPGTESYFIPEVC | 632 | 273 |
| | 173 | 192 | CKMVKLKREKPGRDQNFVILEC | 897 | 274 |
| | 174 | 193 | CMVKLKREKNPGDQNFVILEFC | 1497 | 10 |
| | 176 | 195 | CKLKREKNSRPGNFVILEFPVC | 833 | 275 |
| | 177 | 196 | CLKREKNSRDPGFVILEFPVEC | 817 | 276 |
| | 304 | 323 | CSKVESSRISPGSSIVVNITEC | 839 | 277 |
| | 306 | 325 | CVESSRISKVPGIVVNITELFC | 943 | 13 |
| | 307 | 326 | CESSRISKVSPGVVNITELFSC | 637 | 278 |
| | 353 | 372 | CPPANFTIQKPGTIVSQTQDFC | 739 | 14 |
| | 354 | 373 | CPANFTIQKEPGIVSQTQDFTC | 461 | 279 |
| | 401 | 420 | CQIVVSEMLSPGRISYDAQFEC | 417 | 280 |
| | 402 | 421 | CIVVSEMLSQPGISYDAQFEVC | 414 | 281 |
| | 403 | 422 | CVVSEMLSQPPGSYDAQFEVIC | 635 | 282 |
| | 488 | 507 | CLSEVLRVKVPGPVDEVQISIC | 601 | 283 |
| | 489 | 508 | CSEVLRVKVIPGVDEVQISILC | 649 | 16 |
| | 490 | 509 | CEVLRVKVIAPGDEVQISILSC | 447 | 284 |

^{∗∗}: based on recurring peaks in other screenings.

### Conclusion

Pepscan analysis identified several CD31 epitopes binding to CD38, listed in Table 5. These sCD31 peptides represent promising hits for binding to CD38 and mediating the therapeutic effects observed in Examples 1-7 above.

TABLE 5

List of main CD31 epitopes identified in this study.

| SEQ ID NO: 1 residues | | Epitope candidate | SEQ ID NO: |
|---|---|---|---|
| Start | End | | |
| 74 | 82 | LFYKDDVLF | 130 |
| 90 | 104 | TESYFIPEVRIYDS | 150 |
| 122 | 126 | EYQVL | 137 |
| 174 | 196 | MVKLKREKNSRDQNFVILEFPVE | 151 |
| 259 | 272 | QVTHLAQEFPEIII | 141 |
| 307 | 324 | ESSRISKVSSIVVNITEL | 152 |
| 403 | 411 | VVSEMLSQP | 153 |
| 416 | 420 | DAQFE | 147 |
| 463 | 476 | VFKDNPTEDVEYQS | 154 |

### Example 9

### Materials and Methods

### KD Determination by Surface Plasmon Resonance

The affinity of SEQ ID NO: 6 and SEQ ID NO: 10 for human recombinant CD38 was determined using a Biacore 3000. Binding affinity was measured and reported by the equilibrium dissociation constant (K_{D}). Recombinant human CD38 (Acrobio, ref CD8-H5224) dissolved in milliQ water was used. The following parameters were used:

| | |
|---|---|
| Biacore SPR system | Biacore 3000 |
| Chip CM5 | BR-1000-12 Biacore (Ge Healthcare) |
| Immobilization buffer | 10 mM sodium acetate pH 5.5 (GE Healthcare) |
| Running buffer | HBS-EP pH 7.4 (GE Healthcare) |
| Regeneration buffer | 10 mM glycine-HCl pH 1.5 (GE Healthcare) |
| Binding buffer | HBS-EP pH 7.4 (GE Healthcare) |
| Immobilization | Channel Fc1 (reference channel): |

activation 0.4 M EDC/0.1 M NHS, 70 µL/7 minutes, flow 10 µL/minutes
Channel Fc2 (coated with peptides of interest):
activation 0.4 M EDC/0.1 M NHS 70 µL/7 minutes, flow 10 µL/minutes
immobilization: 25 nM CD38 diluted in NaAC pH 5.0 70 µl/7 minutes, flow 10 µL/minute, inject 1 M ethanolamine-HCl pH 8.4

| | |
|---|---|
| Binding | Protein injection: flow 5 µL/min, contact time 5 minutes in HBS-EP pH 7.4 |
| Regeneration | Injection:10 mM Gly-HCl, pH 1.5, 15 µL/30 seconds |

### Glucose Uptake Assays

### In Human Daudi Cells

Human Daudi cells cultured in 75 cm² flask were centrifuged ( 300 g, 3 minutes), resuspended in warmed PBS supplemented with 10 % goat serum and seeded in 96-well microplate ( 100 µL, 2 × 10⁶ cells/mL) in order to deprive cells from glucose. Cells were treated with test treatments (10 µg/mL) for 1 hour (37° C.). Cells were then incubated with 500 µM of the fluorescent and non-metabolizable glucose analog 2-NBDG ((2R,3R,4S,5R)-3,4,5,6-tetrahydroxy-2-[(7-nitro-2,1,3-benzoxadiazol-4-yl)amino]hexanal; Thermo Fischer) for 30 minutes at 37° C., and washed 4 times with PBS containing 10 % of goat serum. Images were acquired using a Zeiss microscope (20 X) and the relative fluorescence intensity of 30 cells was determined using Fiji.

### In Cat and Dog PBMCs

The same method used for human Daudi cells was used for dog and cat PBMCs, with 200,000 cells plated per well.

### Short High Fat Diet-induced Obesity (HFD) Mouse Model

### Experimental Design

Animals were exposed to normal diet (10% calories from fat) or high fat diet (60% calories from fat) for 3 days. Test compound (SEQ ID NO: 10, 300 µg/kg s.c.) was administered every day starting the day before exposure to HFD. Three days after inception of the HFD regimen, mice were fasted for 5 hours and a glucose challenge was performed.

### Glycaemia Assessment

Glycaemia was measured using a Contour XT glucometer (Ascensia Diabetes Care) and appropriate measurement strips (Contour Next). Fasting glycaemia was first measured (T₀), and then glucose was administered via i.p. injection (2 g/kg). Glycaemia measurements were performed 15, 30, 90, and 120 minutes after i.p. glucose injection. Data were expressed as area under the curve (AUC).

### Determination of Serum Insulin Levels

Blood samples were collected before glucose injection as well as 15 minutes after glucose injection. Sera were obtained by centrifuging blood samples at 2,500 g for 20 minutes at 4° C. and frozen at -80° C. Serum insulin was measured using an ultrasensitive mouse insulin ELISA kit according to the manufacturer’s instructions (Crystal Chem). Data were expressed as area under the curve (AUC).

### Statistical Analysis

The statistical analyses and figures were made using the software Sigma Plot. One-way and post hoc tests were performed, if the conditions of application were respected.

### Short Acute Kidney Injury (AKI) Mouse Model

### Experimental Design

Animals were injected with PBS or folic acid (250 mg/kg dissolved in 0.3 M sodium bicarbonate). Blood samples were collected every day for 3 consecutive days. Sera were obtained by centrifuging blood samples at 2,500 g for 20 minutes at 4° C. and frozen at -80° C.

### Blood Urea Nitrogen (BUN) Levels Assessment

BUN levels were determined using a BUN assay kit (ThermoFischer) according to manufacturer’s instructions.

### Statistical Analysis

The statistical analyses and figures were made using the software Sigma Plot. One-way and post hoc tests were performed, if the conditions of application were respected.

### DSS Mouse Model

### Experimental Design

Experimental colitis was induced in mice by giving drinking water ad libitum containing 3% (w/v) DSS for 5 days, while control mice received tap water only. Test compounds (SEQ ID NO: 6 and SEQ ID NO: 10) were subcutaneously administered everyday (300 µg/kg) starting when DSS treatment began. Mice were sacrificed 9 days after the beginning of DSS treatment.

### Statistical Analysis

The statistical analyses and figures were made using the software Sigma Plot. One-way and post hoc tests were performed, if the conditions of application were respected.

### LPS Mouse Model

### Experimental Design

Mice (n = 5 per group) were daily injected with LPS (700 µg/kg ip) for two weeks. One week after LPS treatment initiation, mice were daily injected with SEQ ID NO: 10 (50 µg/kg s.c.) for one week.

### Glycaemia Assessment

Glycaemia was measured using a Contour XT glucometer (Ascensia Diabetes Care) and appropriate measurement strips (Contour Next). Fasting glycaemia was first measured (T₀), and then glucose was administered via i.p. injection (2 g/kg). Glycaemia measurements were performed 15, 30, 90, and 120 minutes after i.p. glucose injection.

### Determination of Serum Insulin Levels

Blood samples were collected before glucose injection as well as 15 minutes after glucose injection. Sera were obtained by centrifuging blood samples at 2500 g for 20 minutes at 4° C. and frozen at -80° C. Serum insulin was measured using an ultrasensitive mouse insulin ELISA kit according to the manufacturer’s instructions (Crystal Chem).

### Statistical Analysis

The statistical analyses and figures were made using the software Sigma Plot. One-way and post hoc tests were performed, if the conditions of application were respected.

### Results

To determine the effect of test compounds on glucose uptake, we developed a cellular model based on the use of human Daudi cells, a cell line known to strongly express CD38.

Human Daudi cells were plated in culture medium deprived of glucose and exposed to test treatments (peptides of Table 6) for 1 hour and then incubated with the non-metabolizable, fluorescent glucose 2-NBDG. After 30 minutes of incubation, cells were washed and imaged using a microscope. Fluorescence levels were determined using Fiji.

TABLE 6

| SEQ ID NO: 1 residues | | Amino acid sequence | Structure | SEQ ID NO: |
|---|---|---|---|---|
| Start | Stop | | | |
| 90 | 96 | STESYFI | Linear | 4 |
| 90 | 96 | CSTESYFIC | Loop | 5 |
| 182 | 196 | NSRDQNFVILEFPVE | Linear | 6 |
| 187 | 193 | NFVILEF | Linear | 7 |
| 187 | 194 | NFVILEFP | Linear | 8 |
| 184 | 198 | CRDQNFVILEFPVEEQC | Loop | 9 |
| 174 | 193 | CMVKLKREKNPGDQNFVILEFC | β-sheet-like fold | 10 |
| 195 | 201 | CVEEQDRVC | Loop | 11 |
| 317 | 325 | CIVVNITELFC | Loop | 12 |
| 306 | 322 | CVESSRISKVPGIVVNITELFC | β-sheet-like fold | 13 |
| 353 | 372 | CPPANFTIQKPGTIVSQTQDFC | β-sheet-like fold | 14 |
| 470 | 475 | CEDVEYQC | Loop | 15 |
| 489 | 508 | CSEVLRVKVIPGVDEVQISILC | β-sheet-like fold | 16 |
| 582 | 589 | RVILAPWK | Linear | 17 |

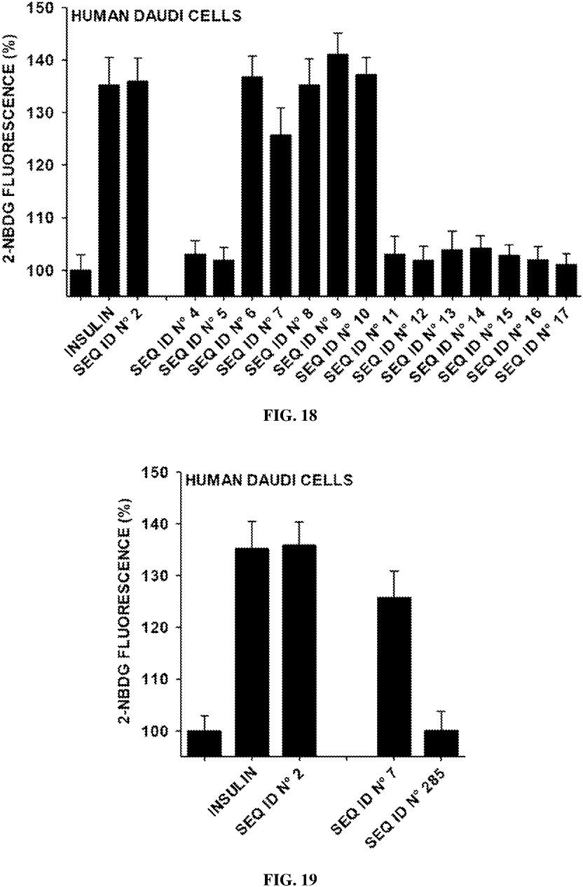

We first observed that human sCD31 (SEQ ID NO: 2) exposure increased glucose uptake to similar levels to that observed in the presence of insulin (FIG. 18).

Noticeably, only compounds comprising the NFVILEF motif (SEQ ID NO: 7) were found to reproduce the ability of human sCD31 to increase glucose uptake (i.e., SEQ ID NOs: 6 to 10) (FIG. 18).

Importantly, the order of amino acid residues in the sequence must be conserved since a scramble peptide containing the same amino acid residues but in a different order (FELIVFN, SEQ ID NO: 285) was not able to increase glucose uptake (FIG. 19).

To determine the minimal pharmacophore responsible for CD31-mediated increase in glucose uptake, we carried out deletions and point mutations, as follows.

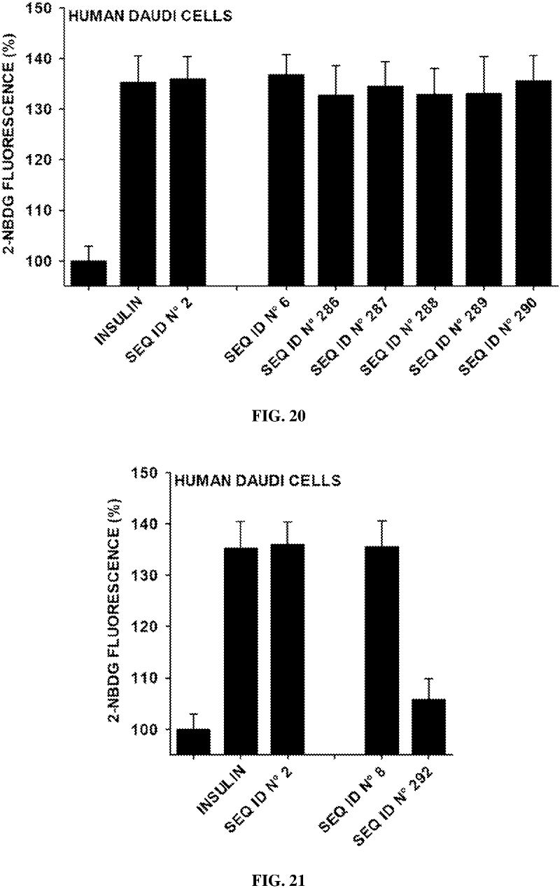

Starting from the NSRDQNFVILEFPVE sequence (SEQ ID NO: 6), we removed C-terminal amino acids one by one (SEQ ID NOs: 286 to 290) and observed that these removals failed to reduce glucose uptake up to the sequence NSRDQNFVIL (SEQ ID NO: 290), suggesting, together with the results of FIG. 18 explained above, that the presence of the NFVIL amino acid sequence (SEQ ID NO: 321) only is sufficient to reproduce the effect of sCD31 (FIG. 20).

Starting from the NFVILEFP sequence (SEQ ID NO: 8), which corresponds to a β-sheet structure in CD31 3-D structure (PDB ID: 5c14, doi: 10.2210/pdb5C14/pdb), we observed that removal of the first asparagine residue in N-term to obtain the sequence FVILEFP (SEQ ID NO: 292) failed to reproduce the effect of sCD31 (FIG. 21), demonstrating that the minimal pharmacophore starts at amino acid N (amino acid residue 187 of SEQ ID NO: 1).

Together, the results of FIG. 18, FIG. 20 and FIG. 21 confirm that the NFVIL amino acid sequence (SEQ ID NO: 321) is the minimal pharmacophore responsible for CD31-mediated increase in glucose uptake.

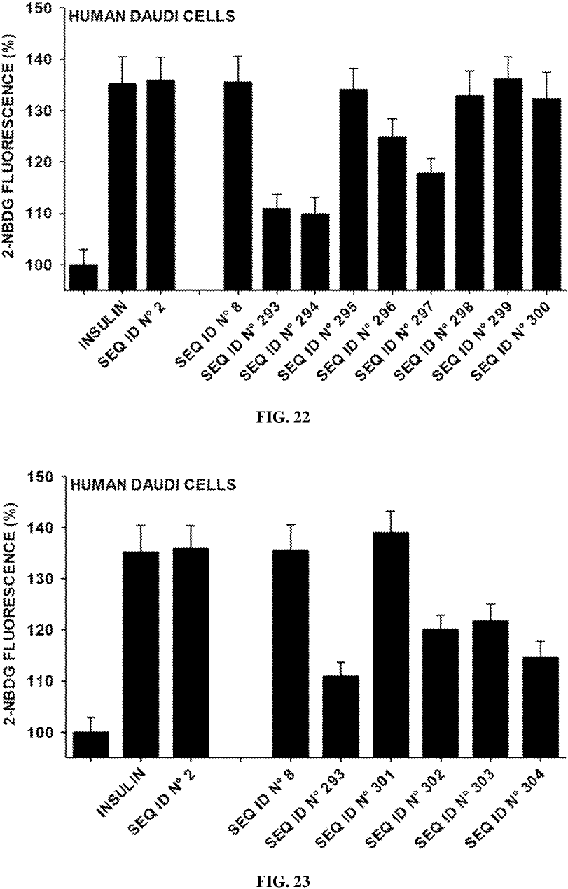

By performing an alanine scanning on the NFVILEFP sequence (SEQ ID NO: 8), we observed that alanine substitutions of the valine residue at position 3 of SEQ ID NO: 8 (SEQ ID NO: 295), of the glutamic acid residue at position 6 of SEQ ID NO: 8 (SEQ ID NO: 298), of the phenylalanine residue at position 7 of SEQ ID NO: 8 (SEQ ID NO: 299), or of the proline residue at position 8 of SEQ ID NO: 8 (SEQ ID NO: 300) had no effect on glucose uptake, further confirming that the presence of NFVIL amino acids (SEQ ID NO: 321) only is sufficient to reproduce the effect of sCD31; and demonstrating that the valine residue at position 3 of SEQ ID NO: 8 (or of SEQ ID NO: 321) can be substituted to any amino acid with no loss of activity (FIG. 22).

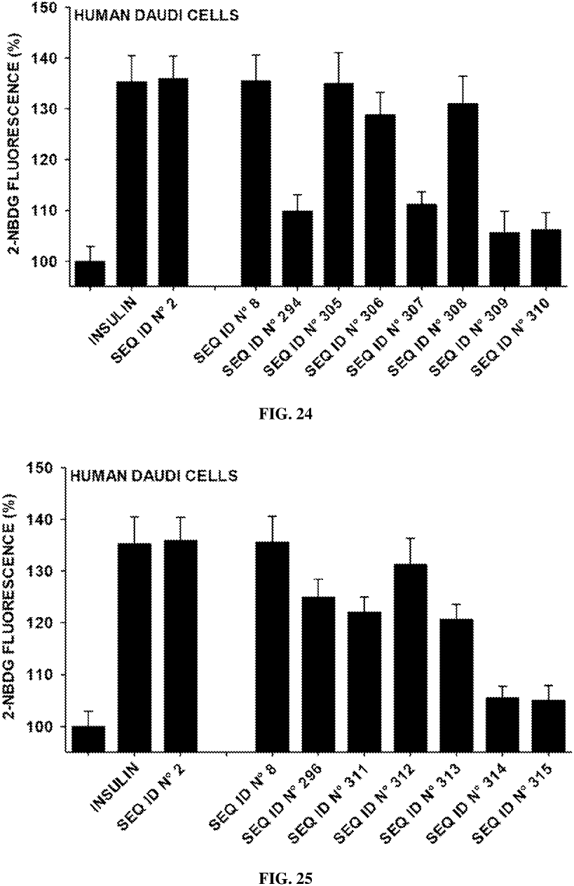

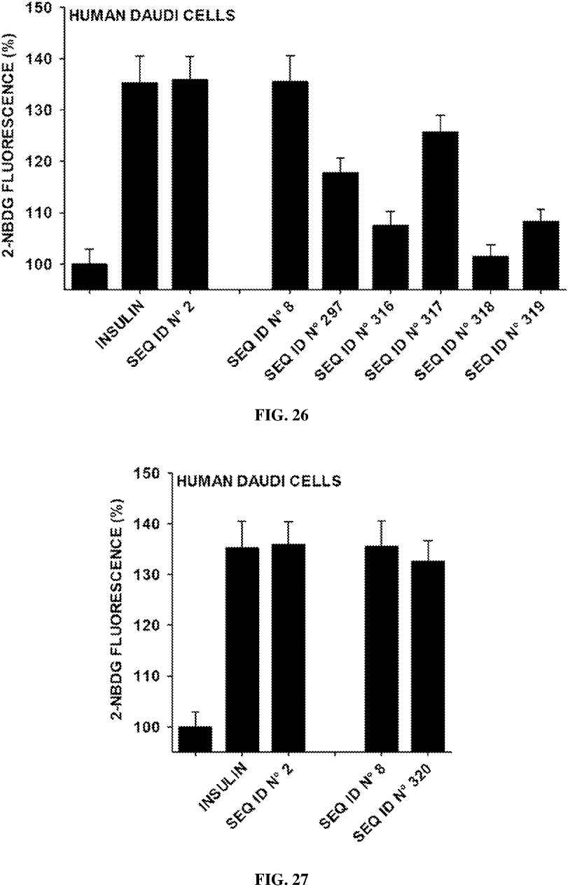

Starting from the NFVILEFP sequence (SEQ ID NO: 8) and to determine possible substitutions on amino acids determined from FIG. 22 by alanine scanning as essential for conserving glucose uptake activity (i.e., the asparagine residue at position 1 of SEQ ID NO: 8, phenylalanine residue at position 2 of SEQ ID NO: 8, isoleucine residue at position 4 of SEQ ID NO: 8, and leucine residue at position 5 of SEQ ID NO: 8), we further observed the following:
Substitutions on position 1 (N) (FIG. 23): substitution by a glutamine residue (QFVILEFP, SEQ ID NO: 301) was found to still increase glucose uptake. Substitutions by a serine residue (SFVILEFP, SEQ ID NO: 302) or a threonine residue (TFVILEFP, SEQ ID NO: 303), all of which - together with glutamine - belong to the same class of amino acids (i.e., amino acids with a polar uncharged side chain), were found to still increase glucose uptake, although to a lesser extent. Substitution by a histidine residue (HFVILEFP, SEQ ID NO: 304), i.e., an amino acid with a positively-charged side chain at physiological pH, was found to impact the glucose uptake-increasing activity of the peptide.
   ➜ These results demonstrate that the asparagine residue at position 1 of SEQ ID NO: 321 can be substituted by an amino acid residue with a polar uncharged side chain (i.e., glutamine, serine or threonine), and preferably by a glutamine residue, without impacting glucose uptake increasing activity.
Substitutions on position 2 (F) (FIG. 24): substitutions by a tyrosine residue (NYVILEFP, SEQ ID NO: 305) or a tryptophan residue (NWVILEFP, SEQ ID NO: 306), two amino acids that belong to the same class (i.e., amino acids with hydrophobic side chains), were found to still increase glucose uptake, but not a substitution by an isoleucine residue (NIVILEFP, SEQ ID NO: 307). Substitution by a serine residue (NSVILEFP, SEQ ID NO: 308), part of the uncharged side chain group of amino acids, was found to keep glucose uptake-increasing activity. However, substitutions by a histidine residue (NHVILEFP, SEQ ID NO: 309), an amino acid with a positively-charged side chain at physiological pH, or by an aspartic acid residue (NDVILEFP, SEQ ID NO: 310), an amino acid with a negatively-charged side chain at physiological pH, were found to impact the glucose uptake-increasing activity of the peptide.
   ➜ These results demonstrate that the phenylalanine residue at position 2 of SEQ ID NO: 321 can be substituted by another aromatic amino acid residue (i.e., a tyrosine or a tryptophan), or by an uncharged polar side chain amino acid residue (i.e., serine, threonine, asparagine or glutamine) without impacting glucose uptake-increasing activity.
Substitutions on position 4 (I) (FIG. 25): we observed in the alanine scanning of FIG. 22 that substitution by an alanine residue (NFVALEFP, SEQ ID NO: 296) slightly decreased the activity of the peptide, although it remained overall active. We observed that substitution by a phenylalanine residue (NFVFLEFP, SEQ ID NO: 311) from the same family of hydrophobic side chain amino acids, as well as substitutions by amino acids with a polar uncharged side chain (threonine: NFVTLEFP, SEQ ID NO: 312; glutamine: NFVQLEFP, SEQ ID NO: 313) were also still active. However, substitutions by a histidine residue (NFVHLEFP, SEQ ID NO: 314), an amino acid with a positively-charged side chain at physiological pH, or by an aspartic acid residue (NFVDLEFP, SEQ ID NO: 315), an amino acid with a negatively-charged side chain at physiological pH, were found to impact the glucose uptake-increasing activity of the peptide.
   ➜ These results demonstrate that the isoleucine residue at position 4 of SEQ ID NO: 321 can be substituted to any amino acid with a hydrophobic side chain (i.e., alanine, valine, leucine, methionine, cysteine, phenylalanine, tyrosine or tryptophan) or an uncharged side chain (serine, threonine, asparagine or glutamine) without impacting glucose uptake-increasing activity.
Substitutions on position 5 (L) (FIG. 26): we observed in the ALA-scan of FIG. 22 that substitution by an alanine residue (NFVIAEFP, SEQ ID NO: 297) slightly decreased the activity of the peptide, although it remained overall active. We observed that substitution by a phenylalanine residue (NFVIFEFP, SEQ ID NO: 317), a residue from the same family as alanine, also increased glucose uptake, even more efficiently than SEQ ID NO: 297. However, substitutions by a threonine residue (NFVITEFP, SEQ ID NO: 316), an amino acid with a polar uncharged side chain, by a histidine residue (NFVIHEFP, SEQ ID NO: 318), an amino acid with a positively-charged side chain at physiological pH, or by an aspartic acid residue (NFVIDEFP, SEQ ID NO: 319), an amino acid with a negatively-charged side chain at physiological pH, were all three found to impact the glucose uptake-increasing activity of the peptide.
   ➜ These results demonstrate that the leucine residue at position 5 of SEQ ID NO: 321 can be substituted to any amino acid with a hydrophobic side chain (i.e., alanine, valine, isoleucine, methionine, cysteine, phenylalanine, tyrosine or tryptophan) without impacting glucose uptake-increasing activity.

Of interest, NSMLIEFT (SEQ ID NO: 320) was still able to increase glucose uptake (FIG. 27), demonstrating that a peptide comprising a combination of several substitutions is still active.

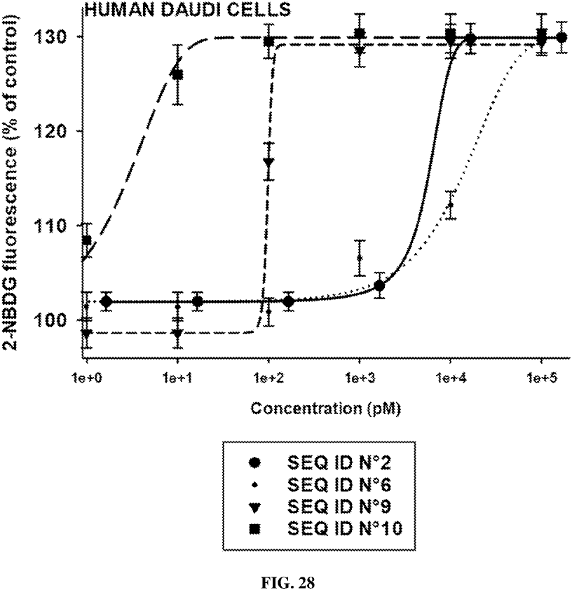

From a structural point of view, we observed that sCD31 (SEQ ID NO: 2) had an EC₅₀ of around 5 nM (FIG. 28). It is interesting to note that NSRDQNFVILEFPVE sequence (SEQ ID NO: 6) had an EC₅₀ of around 13 nM, close to that of sCD31. Surprisingly, the looped peptide CRDQNFVILEFPVEEQC (SEQ ID NO: 9) and β-sheet-like folded CMVKLKREKNPGDQNFVILEFC (SEQ ID NO: 10) had lower EC₅₀ of around 100 pM and 2 pM, respectively.

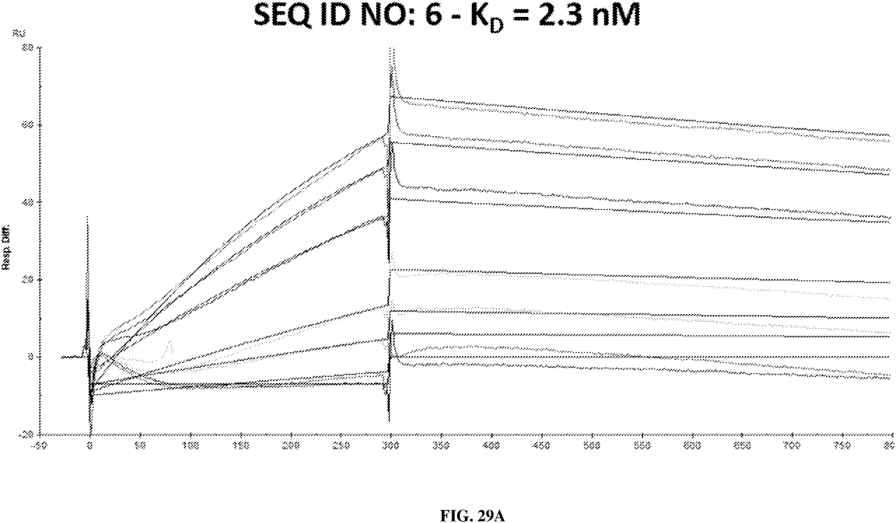

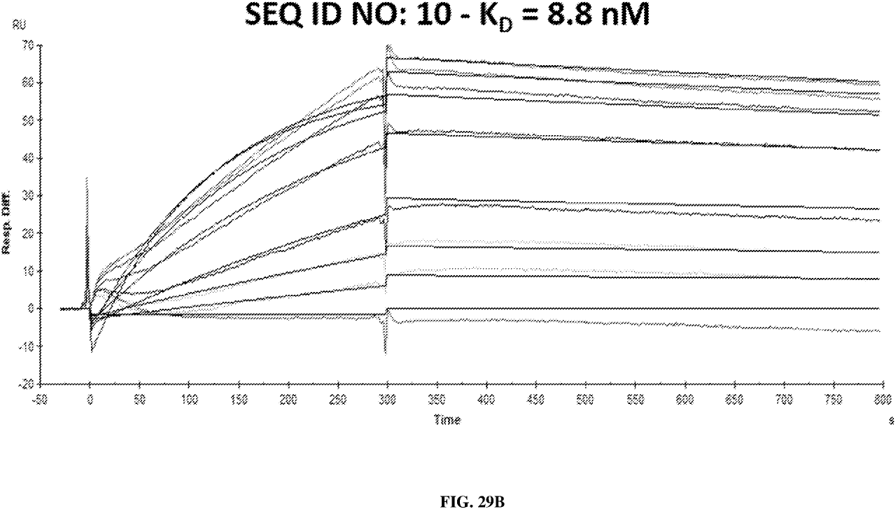

Even more surprisingly, the EC₅₀ improvement conferred by 2-D conformation of the peptide was not associated to increased affinity, since the NSRDQNFVILEFPVE (SEQ ID NO: 6) and CMVKLKREKNPGDQNFVILEFC (SEQ ID NO: 10) peptides had similar affinities for CD38 (2.3 nM and 8.8 nM, respectively), as determined by surface plasmon resonance (FIGS. 29A-B).

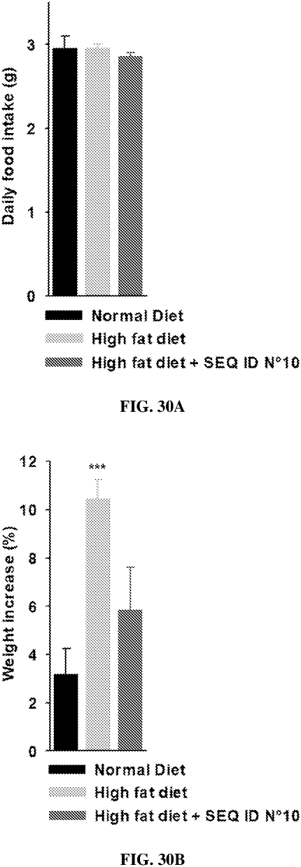

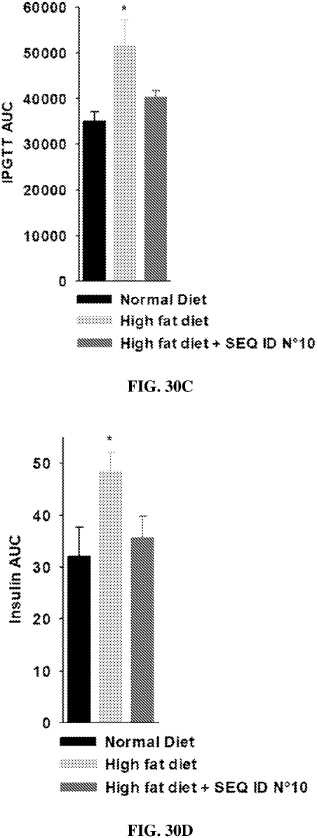

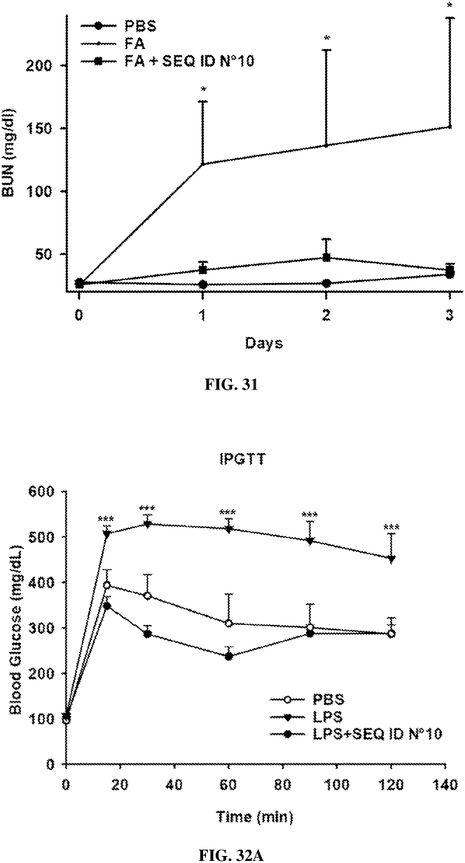

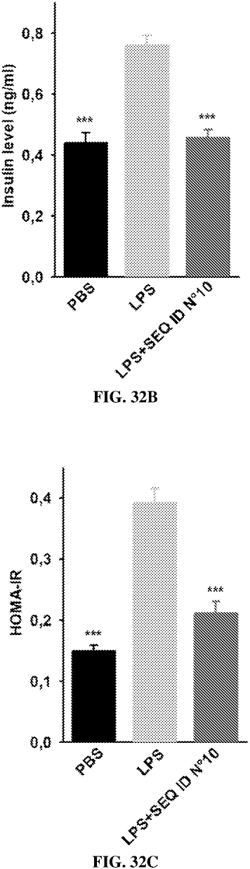

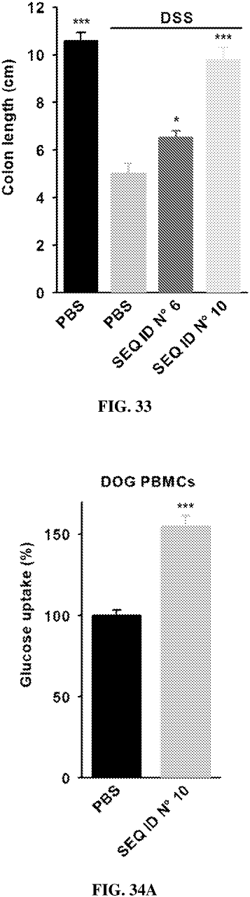

In vivo, the β-sheet-like folded peptide with amino acid sequence CMVKLKREKNPGDQNFVILEFC (SEQ ID NO: 10), injected every day (s.c., 300 pg/kg), was found to:
reduce weight gain, and to normalize glycaemia and insulin response in an HFD mouse model (FIG. 30), thus demonstrating a preventive and curative effect on metabolic syndrome, and in particular on obesity and diabetes;
ameliorate kidney function in a folic acid-induced acute kidney injury model, an effect assessed by the measurement of blood urea nitrogen levels (FIG. 31), thus demonstrating an effect against acute kidney injury (AKI) and acute renal failure (ARF);
normalize blood glucose and insulin levels and maintain insulin sensitivity in an LPS-induced type 2 diabetes (T2DM) mouse model (FIGS. 32A-C), thus demonstrating anti-diabetic properties;
protect gut from shortening in the DSS mouse model (FIG. 33), thus demonstrating a preventive and curative effect on inflammatory bowel disease and in particular ulcerative colitis.

In this latter model, it is interesting to note that SEQ ID NO: 6, while demonstrating a statistically significant improvement in colon length, was less potent than SEQ ID NO: 10 when injected at the same dosing regimen (300 µg/kg s.c. daily). This confirms the results of FIG. 28, i.e., that 2-D structured peptides are more active than linear peptides, and would therefore need to be administered in lesser amounts to achieve the same therapeutic effect than their linear counterparts.

Finally, a multiple sequence alignment of the amino acid sequences of full length CD31 from Homo sapiens (SEQ ID NO: 1), Mus musculus (SEQ ID NO: 323), Rattus norvegicus (SEQ ID NO: 324), Felis catus (SEQ ID NO: 325), Canis lupus (SEQ ID NO: 326), Ovis aries (SEQ ID NO: 327), Sus scrofa (SEQ ID NO: 328), Bos taurus (SEQ ID NO: 329), and Gallus gallus (SEQ ID NO: 330) confirms a high conservation of the minimal pharmacophore identified herein in human CD31, and together with the data presented above, suggests that these homologous minimal pharmacophores are also capable of binding to CD38 and thereby trigger a biological activity:

NFVIL (SEQ ID NO: 321) in Homo sapiens;
NFVLM (SEQ ID NO: 331) in Mus musculus;
NFVLI (SEQ ID NO: 332) in Rattus norvegicus;
NFMVM (SEQ ID NO: 333) in Felis catus;
NFMML (SEQ ID NO: 334) in Canis lupus;
NFVTL (SEQ ID NO: 335) in Ovis aries;
NSVTL (SEQ ID NO: 336) in Sus scrofa;
NFVTL (SEQ ID NO: 335) in Bos taurus; and
NFSVV (SEQ ID NO: 337) in Gallus gallus.

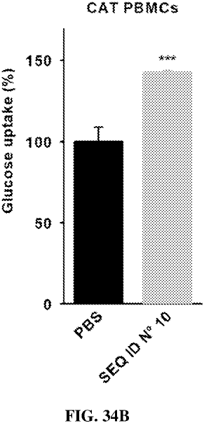

This is further supported by the good cross-reactivity observed with SEQ ID NO: 10 across species, since the glucose uptake-increasing activity of this peptide (derived from human sCD31) was also observed in cat and dog PBMCs (FIGS. 34A-B respectively).

1-25. (canceled)

26. An isolated peptide which specifically binds to CD38, and which comprises an amino acid sequence X₁-X₂-X₃-X₄-X₅ with SEQ ID NO: 322, wherein:
X₁ is an amino acid residue with a polar uncharged side chain,
X₂ is an aromatic amino acid residue or an amino acid residue with a polar uncharged side chain,
X₃ is any amino acid residue,
X₄ is an amino acid residue with a hydrophobic side chain or with a polar uncharged side chain, and
X₅ is an amino acid residue with a hydrophobic side chain,
with the proviso that the isolated peptide does not comprise or consist of the full CD31^{Ig-like} ¹⁻² domains consisting of amino acid residues 35 to 233 of SEQ ID NO: 1, and
wherein said peptide does not consist of any one of SEQ ID NOs: 338, 339, 340 or 341.

27. The isolated peptide according to claim 26, wherein said isolated peptide specifically binds to human CD38.

28. The isolated peptide according to claim 26, wherein:
X₁ is selected from the group consisting of asparagine (Asn, N), glutamine (Gln, Q), serine (Ser, S), and threonine (Thr, T),
X₂ is selected from the group consisting of phenylalanine (Phe, F), tyrosine (Tyr, Y), tryptophan (Trp, W), serine (Ser, S), threonine (Thr, T), asparagine (Asn, N), and glutamine (Gln, Q);
X₃ is any amino acid residue,
X₄ is selected from the group consisting of isoleucine (Ile, I), alanine (Ala, A), valine (Val, V), leucine (Leu, L), methionine (Met, M), cysteine (Cys, C), phenylalanine (Phe, F), tyrosine (Tyr, Y), tryptophan (Trp, W), serine (Ser, S), threonine (Thr, T), asparagine (Asn, N), and glutamine (Gln, Q), and
X₅ is selected from the group consisting of leucine (Leu, L), alanine (Ala, A), valine (Val, V), isoleucine (Ile, I), methionine (Met, M), cysteine (Cys, C), phenylalanine (Phe, F), tyrosine (Tyr, Y), and tryptophan (Trp, W).

29. The isolated peptide according to claim 28, wherein X₃ is valine (Val, V).

30. The isolated peptide according to claim 26, wherein the isolated peptide is linear.

31. The isolated peptide according to claim 30, wherein the isolated peptide comprises an amino acid sequence selected from the group consisting of the sequences of Table 1.

32. The isolated peptide according to claim 26, wherein said isolated peptide is a 2-D structured peptide.

33. The isolated peptide according to claim 32, wherein the 2-D structured peptide is cyclized and/or beta-turned.

34. The isolated peptide according to claim 32, wherein the 2-D structured peptide is cyclized by disulfide bridge formation, head-to-tail cyclization, side-chain-to-side-chain cyclization, head-to-side-chain cyclization, side-chain-to-tail cyclization, thioether or ether bridge formation, lactone or thiolactone bridge formation, or lactam bridge formation.

35. The isolated peptide according to claim 32, wherein the isolated peptide is cyclized and comprises an amino acid sequence selected from the group consisting of the sequences of Table 2.

36. The isolated peptide according to claim 32, wherein the isolated peptide is beta-turned and comprises an amino acid sequence selected from the group consisting of the sequences of Table 3.

37. The isolated peptide according to claim 26, wherein the isolated peptide comprises an amino acid sequence selected from the group consisting of SEQ ID NOs: 6-10, 286-290, 295-303, 305, 306, 308, 311-313, 317, 320, 321, and 331-337.

38. The isolated peptide according to claim 26, wherein the isolated peptide comprises an amino acid sequence selected from the group consisting of SEQ ID NOs: 6-10, 286-290, 295, 296, 298-301, 305, 306, 308, 311-313, 317, 320, 321, and 331-337.

39. The isolated peptide according to claim 26, wherein the isolated peptide comprises an amino acid sequence selected from the group consisting of SEQ ID NOs: 6, 8-10, 286-290, 295, 298-301, 305, 306, 308, 312, and 320.

40. The isolated peptide according to claim 26, wherein said peptide comprises at most 150 amino acid residues.

41. The isolated peptide according to claim 26, wherein said peptide is fused to a payload being a therapeutic or diagnostic payload and/or a carrier payload.

42. A method of preventing and/or treating a disease characterized by increased levels of soluble CD38 in a subject and/or a reduced soluble CD31 level/soluble CD38 level ratio in a subject in need thereof, comprising administering to said subject the isolated peptide according to claim 26, or a conjugate comprising the isolated peptide fused to a payload being a therapeutic or diagnostic payload and/or a carrier payload.

43. A method of preventing and/or treating a disease selected from inflammatory diseases; autoimmune diseases; metabolic and endocrine diseases; neurodegenerative diseases; neuroinflammatory diseases; cardiovascular diseases; kidney diseases; diabetic complications; ocular diseases; age-related diseases; fibrotic disorders; and cancer and metastasis; in a subject in need thereof, comprising administering to said subject the isolated peptide according to claim 26, or a conjugate comprising the isolated peptide fused to a payload being a therapeutic or diagnostic payload and/or a carrier payload.

44. The method according to claim 43, wherein said disease is selected from diabetic complications; rheumatoid arthritis; systemic lupus erythematosus; diabetes; obesity; non-alcoholic steatohepatitis; chronic kidney disease; age-related macular degeneration; and glaucoma.

45. A method of increasing the level of at least one anti-inflammatory cytokine in a subject in need thereof, comprising administering to said subject the isolated peptide according to claim 26, or a conjugate comprising the isolated peptide fused to a payload being a therapeutic or diagnostic payload and/or a carrier payload.

46. The method according to claim 45, wherein the method is for increasing the level of at least one anti-inflammatory cytokine in the blood of said subject.

47. The method according to claim 45, wherein said anti-inflammatory cytokine is interleukin-10 (IL-10).

48. A method of lowering glucose levels in a subject in need thereof, comprising administering to said subject the isolated peptide according to claim 26, or a conjugate comprising the isolated peptide fused to a payload being a therapeutic or diagnostic payload and/or a carrier payload.

49. The method according to claim 48, wherein the method is for lowering glucose levels in the blood of said subject.

50. A method of reducing the severity of fibrosis and/or slowing down the progression of fibrosis and/or preventing the development of fibrosis in a subject in need thereof, comprising administering to said subject the isolated peptide according to claim 26 or a conjugate comprising the isolated peptide fused to a payload being a therapeutic or diagnostic payload and/or a carrier payload.

51. A method of promoting or enhancing reparation of DNA lesions induced by a chemotherapeutic agent in a subject in need thereof, comprising co-administering to said subject (i) said chemotherapeutic agent and (ii) the isolated peptide according to claim 26, or a conjugate comprising the isolated peptide fused to a payload being a therapeutic or diagnostic payload and/or a carrier payload, and/or administering to said subject a conjugate comprising said isolated peptide fused to a payload, the conjugate’s payload being said chemotherapeutic agent.
